(19) United States
(12) Patent Application Publication
(10) Pub. No.: US 2025/0049813 A1
(43) Pub. Date: Feb. 13, 2025

(54) CERTAIN N-(1-CYAN0-2-PHENYLETHYL]-1,4-OXAZEPANE-2-CARBOXAMIDES FOR TREATING CHRONIC RHINOSINUSITIS

(71) Applicant: Insmed Incorporated, Bridgewater, NJ (US)

(72) Inventors: Ariel TEPER, Bridgewater, NJ (US); Carlos FERNANDEZ, Bridgewater, NJ (US); David CIPOLLA, Bridgewater, NJ (US)

(21) Appl. No.: 18/704,872

(22) PCT Filed: Oct. 28, 2022
(86) PCT No.: PCT/US2022/048249

§ 371 Date: Apr. 25, 2024

Related U.S. Application Data
(60) Provisional application No. 63/273,540, filed on Oct. 29, 2021.

(51) Int. Cl. A61K 31/553; A61K 31/58; A61P 11/02

(52) U.S. Cl. CPC A61K 31/553; A61K 31/58; A61P 11/02

(57) ABSTRACT

The present disclosure relates to methods for treating chronic rhinosinusitis with a composition comprising an effective amount of a N-(1-cyano-2-phenylethyl)-1,4-oxazepane-2-carboxamide DPP1 inhibitor compound of Formula (I) or a pharmaceutically acceptable salt thereof. In one embodiment, the compound of Formula (I) is (2S)-N-{(1S)-1-cyano-2-[4-(3-methyl-2-oxo-2,3-dihyd ro-1,3-benzoxazol-5-yl)phenyl]ethyl}-1,4-oxazepane-2-carboxamide (brensocatib).

### CROSS REFERENCE TO RELATED APPLICATION

This application claims priority from U.S. Provisional Application Ser. No. 63/273,540, filed Oct. 29, 2021, the disclosure of which is incorporated by reference herein in its entirety.

### BACKGROUND OF THE INVENTION

Chronic rhinosinusitis (CRS) is a common upper respiratory condition affecting the physical and mental health of more than 50 million people worldwide. Patients with CRS may have symptoms such as, nasal congestion, facial pain or pressure, nasal obstruction, nasal discharge, and loss of smell, lasting 12 weeks or longer. Thus, CRS results in reduced quality of life due to its adverse effects on health, vitality, and social functioning, and is also associated with increased rates of depression and anti-depressant use.

Current treatments include intranasal or oral steroids, antihistamines, oral antibiotics, and endoscopic sinus surgery (ESS). While a minor proportion (about 20%) of CRS patients develop nasal polyps (a condition referred to as chronic rhinosinusitis with nasal polyps or CRSwNP), most patients with CRS do not develop nasal polyps and have chronic rhinosinusitis without nasal polyps (CRSsNP). Chronic rhinosinusitis, particularly CRSsNP, is associated with a skewed type-1 immune response with a predominance of neutrophilic inflammation, which can affect the treatment outcome of CRS patients. Neutrophilia is associated with poor response to corticosteroid therapy.

CRSsNP is the most common type of rhinosinusitis and is characterized by a chronic inflammation of the sinonasal mucosa and paranasal sinuses. The most common symptoms include nasal congestion, mucus discharge from the nose or mucus that drips down the back of the throat, facial pain, and a decreased sense of smell. Currently, there are no approved therapies for CRSsNP, leaving a significant unmet medical need.

Thus, there is an immediate need to develop effective interventions for patients with chronic rhinosinusitis, particularly refractory chronic rhinosinusitis as well as CRSsNP.

### SUMMARY

The disclosure, in one aspect, provides methods of treating chronic rhinosinusitis (CRS), comprising: administering to the subject for an administration period, a pharmaceutical composition comprising an effective amount of a compound of Formula (I) or a pharmaceutically acceptable salt thereof,

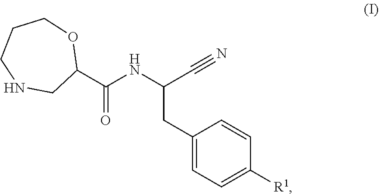

wherein
R¹ is

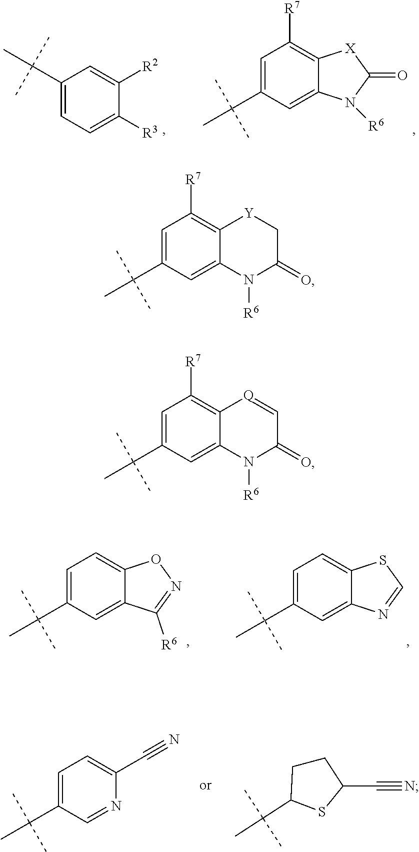

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

In one embodiment of the method provided herein, the CRS is CRS without nasal polyps (CRSsNP). In a further embodiment, the subject is a CRSsNP patient with an eosinophil count <300 cells/μL prior to the administration period. In another embodiment, the CRS is refractory CRSsNP, e.g., steroid-refractory CRSsNP.

In one embodiment, the method for treating CRS comprises decreasing a Lund-Mackay score of the subject during or subsequent to the administration period, as compared to a Lund-Mackay score of the subject prior to the administration period.

In another embodiment, the method comprises decreasing a rhinoscopy sum score of the subject during or subsequent to the administration period, as compared to a rhinoscopy sum score of the subject prior to the administration period.

In another embodiment, the method comprises decreasing a Sino-Nasal Outcome Test-22 (SNOT-22) score of the subject during or subsequent to the administration period, as compared to a SNOT-22 score of the subject prior to the administration period.

In yet another embodiment, the method comprises increasing a University of Pennsylvania Smell Identification Test (UPSIT) score of the subject during or subsequent to the administration period, as compared to a UPSIT score of the subject prior to the administration period.

In even another embodiment, the method comprises decreasing a modified Lund-Kennedy (MLK) score of the subject during or subsequent to the administration period, as compared to a MLK score of the subject prior to the administration period.

In one embodiment, the method comprises decreasing a composite severity score of two or more CRS symptoms of the subject during or subsequent to the administration period, as compared to a composite severity score of the subject prior to the administration period.

In one embodiment, the method comprises decreasing a sinus total symptom score (sTSS) of the subject during or subsequent to the administration period, as compared to an sTSS of the subject prior to the administration period.

In one embodiment, the method comprises decreasing a nasal congestion score (NCS) of the subject during or subsequent to the administration period, as compared to an NCS of the subject prior to the administration period.

In one embodiment, the method comprises decreasing an anterior/posterior rhinorrhea severity score of the subject during or subsequent to the administration period, as compared to an anterior/posterior rhinorrhea severity score of the subject prior to the administration period.

In one embodiment, the method comprises decreasing a facial pain/pressure severity score of the subject during or subsequent to the administration period, as compared to a facial pain/pressure severity score of the subject prior to the administration period.

In one embodiment, the method comprises decreasing a Visual Analog Scale (VAS) score of the subject during or subsequent to the administration period, as compared to a VAS score of the subject prior to the administration period.

In one embodiment, the method comprises increasing a Peak Nasal Inspiratory Flow (PNIF) of the subject during or subsequent to the administration, as compared to a PNIF of the subject prior to the administration period.

In one embodiment, the method comprises decreasing a percentage of sinus opacification of the subject as measured by CT scan volumetry during or subsequent to the administration period, as compared to a percentage of sinus opacification of the subject prior to the administration period.

In one embodiment, the method comprises improving a Patient Global Impression of Severity (PGI-S) score or a Patient Global Impression of Change (PGI-C) score of the subject during or subsequent to the administration period, as compared to a PGI-S score or a PGI-C score of the subject prior to the administration period.

In one embodiment of the methods provided herein, the compound of Formula (I) is an S,S diastereomer, i.e., the compound of Formula (I) has the following stereochemistry:

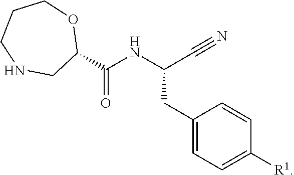

In some embodiments, R¹ is

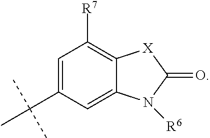

In a further embodiment, X is O; R⁶ is C₁₋₃alkyl; and R⁷ is hydrogen.

In some embodiments, the compound of Formula (I) is (2S)-N-{(1S)-1-cyano-2-[4-(3-methyl-2-oxo-2,3-dihydro-1,3-benzoxazol-5-y-1)phenyl]ethyl}-1,4-oxazepane-2-carboxamide, referred to herein by its international nonproprietary name (INN), brensocatib (and formerly known as INS1007 and AZD7986):

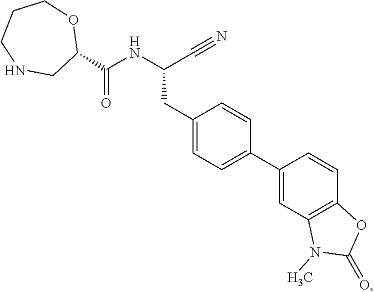 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment, the pharmaceutical composition is administered once-a-day during the administration period.

In one embodiment, the pharmaceutical composition is administered orally during the administration period.

In one embodiment, the compound of Formula (I) is present in the pharmaceutical composition from about 1 mg to about 100 mg, e.g., about 10 mg, about 25 mg, or about 40 mg. In a further embodiment, the compound of Formula (I) is brensocatib.

In one embodiment, the administration period is the entire life of the subject following the initial diagnosis of CRS. In another embodiment, the administration period from about 1 year to about 50 years, from about 1 year to about 30 years, or from about 1 year to about 10 years.

### BRIEF DESCRIPTION OF THE FIGURE

FIG. 1 is a schematic diagram of the study design set forth in Example 1.

### DETAILED DESCRIPTION

The disclosure provides methods for using reversible inhibitors of dipeptidyl peptidase 1 (DPP1), also known as cathepsin C, for treating chronic rhinosinusitis, such as chronic rhinosinusitis without nasal polyps (CRSsNP). DPP1 catalyses excision of dipeptides from the N-terminus of protein and peptide substrates. Through this enzymatic function, DPP1 activates many serine proteases in immune/inflammatory cells, such as, neutrophil serine proteases (NSPs), including neutrophil elastase (NE), proteinase 3 (PR3), cathepsin G (CatG), and neutrophil serine protease 4 (NSP4). Neutrophil serine proteases can mediate neutrophilic inflammation, which is often seen in patients with chronic rhinosinusitis, e.g., CRSsNP. Thus, in some embodiments, the methods of use are methods of treating chronic rhinosinusitis (CRS), e.g., CRSsNP. In a further embodiment, the CRS treated is steroid-refractory CRS, e.g., steroid-refractory CRSsNP.

Without wishing to be bound by a theory, it is thought that the inhibition of DPP1 function by the disclosed DPP1 inhibitors can result in the inhibition of NSPs and/or reduce neutrophilic inflammation, and thus, treat chronic rhinosinusitis. In inadequately controlled CRS, e.g., CRSsNP, neutrophils are the main driver of the remodeling and inflammatory process. Because targeting the activation and infiltration of neutrophils may offer potential therapeutic approaches to improve clinical outcomes in patients with steroid-refractory CRS characterized by neutrophilic or mixed eosinophilic-neutrophilic inflammation (see Ahern and Cervin (2019). Medicina (Kaunas) 55(4), 95; Cho et al. (2016). J Allergy Clin Immunol Pract. 4(4), 575-582; De Greve et al. (2017). Clin Transl Allergy. 7, 22; and Delemarre et al. (2020). J Allergy Clin Immunol. 146(2), 337-43.e6; each of which is incorporated herein by reference in its entirety), inhibition of DPP1 by administering the disclosed DPP1 inhibitors can decrease NSP activity, resulting in decreased neutrophil-mediated inflammation, tissue damage, and mucus production in patients with CRSsNP and leading to an improvement of symptoms (e.g., nasal congestion, facial pain, nasal discharge) and a meaningful decrease of the inflammatory cascade.

Unless defined otherwise, all technical and scientific terms used herein have the meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally, the term “about”, as used herein in references to a measurable value such as an amount of weight, time, dose, etc. is meant to encompass values within an acceptable degree of variability in the art. In some embodiments, degree of variability is based on FDA guidelines.

Also as used herein, “and/or” refers to and encompasses any and all possible combinations of one or more of the associated listed items, as well as the lack of combinations when interpreted in the alternative (“or”).

As used herein, “C₁₋₃” means a carbon group having 1, 2 or 3 carbon atoms.

The term “alkyl”, unless otherwise noted, includes both straight and branched chain alkyl groups and may be substituted or non-substituted. “Alkyl” groups include, but are not limited to, methyl, ethyl, n-propyl, i-propyl, butyl, pentyl.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

As used herein, “treatment” or “treating,” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression, or reducing the severity of the state, disorder or condition or at least one of its clinical or subclinical symptoms).

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant. In one embodiment, the subject is ≥18 to ≤85 years of age.

In one aspect, a method is provided for inhibiting dipeptidyl peptidase (DPP1) in a subject having chronic rhinosinusitis (CRS) or at risk of developing chronic rhinosinusitis, comprising: administering to the subject for an administration period, a pharmaceutical composition comprising an effective amount of a compound of Formula (I). In some embodiments, the DPP1 is expressed by neutrophils. In some embodiments, the administration results in the delay in the onset of CRS in the subject.

In another aspect, methods of treating chronic rhinosinusitis in a subject in need thereof are provided. The method comprises in one embodiment, administering to the subject for an administration period, a pharmaceutical composition comprising an effective amount of a compound of Formula (I). In a further embodiment, the method comprises reducing neutrophilic inflammation in the subject.

In some embodiments, the CRS is CRS without nasal polyps (CRSsNP). In some embodiments, the CRS is CRS with nasal polyps (CRSwNP). In some embodiments, the CRS is refractory CRS. In some embodiments, the refractory CRS is refractory CRS without nasal polyps (CRSsNP). In some embodiments, the refractory CRS is refractory CRS with nasal polyps (CRSwNP).

In one aspect, a method of treating CRS in a subject in need thereof is provided. The method comprises administering to the subject, for an administration period, a pharmaceutical composition comprising an effective amount of a compound of Formula (I), or a pharmaceutically acceptable salt thereof:

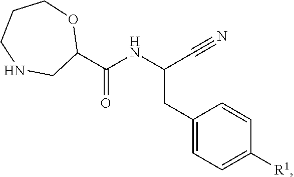

wherein,
R¹ is

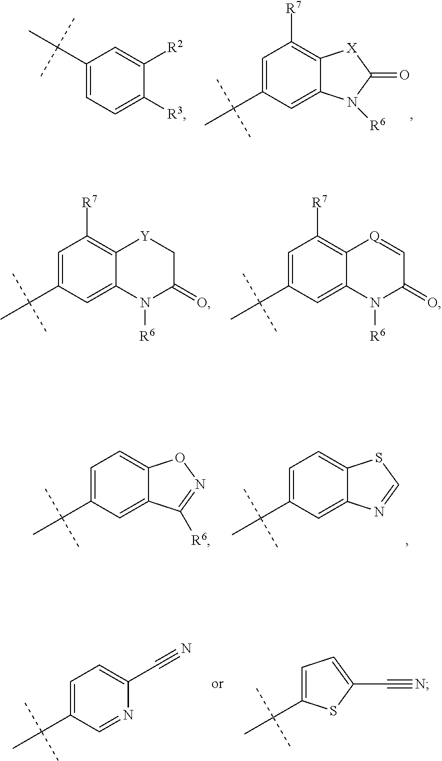

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

In one embodiment of a method provided herein, the compound of Formula (I) is an S,S diastereomer. In other words, the compound of Formula (I) has the following stereochemistry:

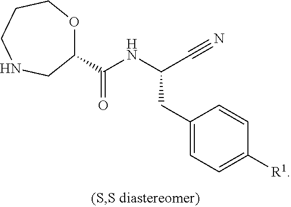

The other diastereomeric forms are also contemplated by the present invention. For example, in one embodiment, the compound of Formula (I) is the R,R diastereomer:

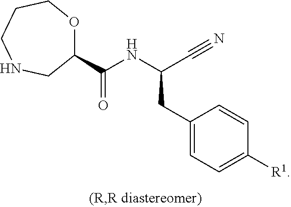

In another embodiment, the compound of Formula (I) is the R,S diastereomer:

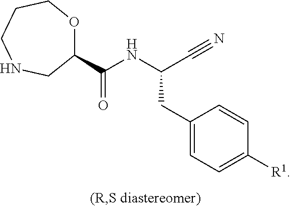

In even another embodiment, the compound of Formula (I) is the S,R diastereomer:

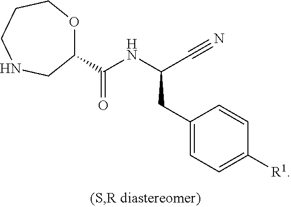

In one embodiment of a method provided herein, R¹ is

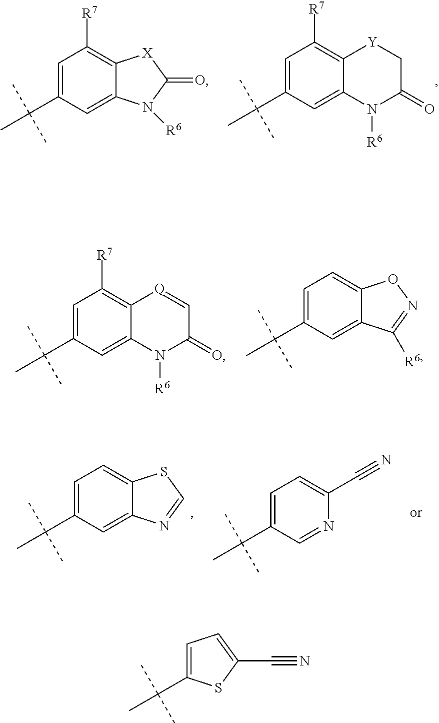

X is O, S or CF₂; Y is O or S; Q is CH or N; R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and R⁷ is hydrogen, F, Cl or CH₃.

In one embodiment R¹ is

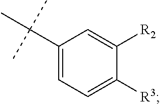

R² is hydrogen, F, Cl, Br, OSO₂C₁₋₃alkyl, or C₁₋₃alkyl; R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring.

In a further embodiment, R¹ is

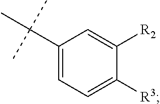

R² is hydrogen, F, Cl or C₁₋₃alkyl; and R³ is hydrogen, F, Cl, CN or SO₂C₁₋₃alkyl.

In still a further embodiment, R¹ is

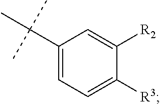

R² is hydrogen, F or C₁₋₃alkyl; and R³ is hydrogen, F or CN.

In another embodiment, R¹ is

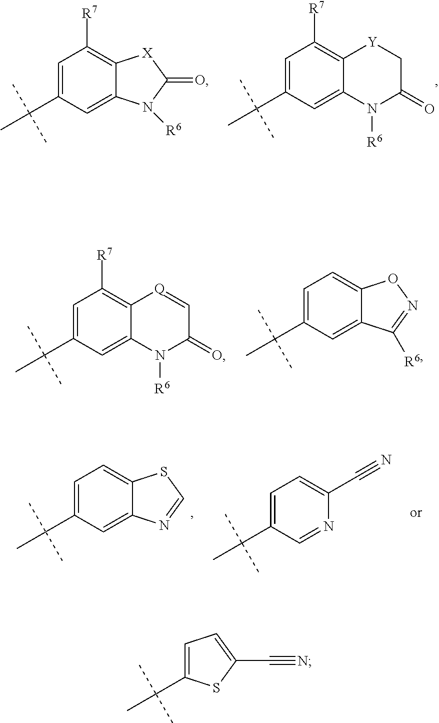

X is O, S or CF₂; Y is O or S; Q is CH or N; R⁶ is C₁₋₃alkyl, wherein the C₁₋₃alkyl is optionally substituted by 1, 2 or 3 F and/or optionally substituted by OH, OC₁₋₃alkyl, N(C₁₋₃ alkyl)₂, cyclopropyl, or tetrahydropyran; and R⁷ is hydrogen, F, Cl or CH₃.

In one embodiment, R¹ is

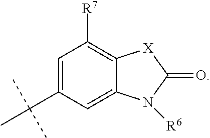

In another embodiment, R¹ is

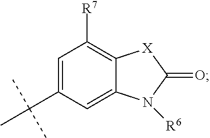

X is O; R⁶ is C₁₋₃alkyl; and R⁷ is hydrogen. In a further embodiment, R⁶ is methyl.

In one embodiment, R¹ is

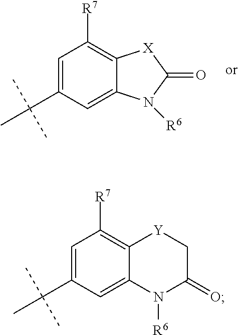

X is O, S or CF₂; Y is O or S; R⁶ is C₁₋₃alkyl, optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃.

In one embodiment, R¹ is

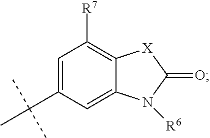

X is O, S or CF₂; R⁶ is C₁₋₃alkyl, wherein the C₁₋₃alkyl is optionally substituted by 1, 2 or 3 F; and R⁷ is hydrogen, F, Cl or CH₃.

In one embodiment, X is O; R⁶ is C₁₋₃alkyl; and R⁷ is hydrogen.

In one embodiment, R¹ is

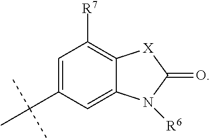

In a further embodiment, X is O; R⁶ is C₁₋₃alkyl; and R⁷ is hydrogen.

In one embodiment, R¹ is

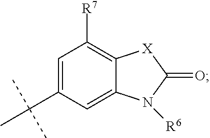

R⁶ is C₁₋₃alkyl; and R⁷ is hydrogen.

In another embodiment, R¹ is

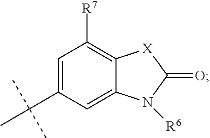

X is O; R⁶ is C₁₋₃alkyl, wherein the C₁₋₃ alkyl is optionally substituted by 1, 2 or 3 F; and R⁷ is hydrogen.

In one embodiment, R² is hydrogen, F, Cl, Br, OSO₂C₁₋₃alkyl or C₁₋₃alkyl.

In a further embodiment, R² is hydrogen, F, Cl or C₁₋₃alkyl.

In still a further embodiment, R² is hydrogen, F or C₁₋₃alkyl.

In one embodiment, R³ is hydrogen, F, Cl, Br, CN, CF₃, SO₂C₁₋₃alkyl CONH₂ or SO₂NR⁴R⁵, wherein R⁴ and R⁵ together with the nitrogen atom to which they are attached form an azetidine, pyrrolidine or piperidine ring.

In a further embodiment, R³ is hydrogen, F, Cl, CN or SO₂C₁₋₃alkyl.

In still a further embodiment, R³ is hydrogen, F or CN.

In one embodiment, R⁶ is C₁₋₃alkyl, wherein said C₁₋₃alkyl is optionally substituted by 1, 2 or 3 F and optionally by one substituent selected from the group consisting of OH, OC₁. 3alkyl, N(C₁₋₃alkyl)₂, cyclopropyl, and tetrahydropyran.

In a further embodiment, R⁶ is C₁₋₃alkyl, wherein said C₁₋₃alkyl is optionally substituted by 1, 2 or 3 F. In still a further embodiment, R⁶ is methyl or ethyl. In still a further embodiment, R⁶ is methyl.

In one embodiment, R⁷ is hydrogen, F, Cl or CH₃. In a further embodiment R⁷ is hydrogen.

In one embodiment of the methods provided herein, the composition administered to the patient comprises an effective amount of (2S)-N-{(1S)-1-cyano-2-[4-(3-methyl-2-oxo-2,3-dihydro-1,3-benzoxazol-5-yl)phenyl]ethyl}-1,4-oxazepane-2-carboxamide (referred to herein by its international nonproprietary name (INN) brensocatib):

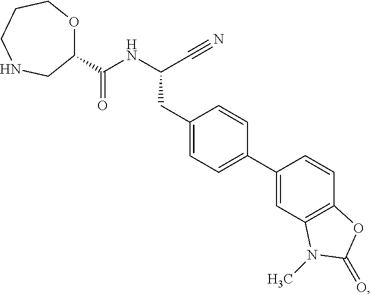 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment, the compound of Formula (I) is:
(2S)-N-[(1S)-1-Cyano-2-(4′-cyanobiphenyl-4-yl)ethyl]-1,4-oxazepane-2-carboxamide,
(2S)-N-{(1S)-1-Cyano-2-[4-(3-methyl-2-oxo-2,3-dihydro-1,3-benzoxazol-5-yl)phenyl]ethyl}-1,4-oxazepane-2-carboxamide,
(2S)-N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide,
4′-[(2S)-2-Cyano-2-{[(2S)-1,4-oxazepan-2-ylcarbonyl]amino}ethyl]biphenyl-3-yl methanesulfonate,
(2S)-N-{(1S)-1-Cyano-2-[4-(3-methyl-1,2-benzoxazol-5-yl)phenyl]ethyl}-1,4-oxazepane-2-carboxamide,
(2S)-N-{(1S)-1-Cyano-2-[4′-(trifluoromethyl)biphenyl-4-yl]ethyl}-1,4-oxazepane-2-carboxamide,
(2S)-N-[(15)-1-Cyano-2-(3′,4′-difluorobiphenyl-4-yl)ethyl]-1,4-oxazepane-2-carboxamide,
(2S)-N-{(15)-1-Cyano-2-[4-(6-cyanopyridin-3-yl)phenyl]ethyl}-1,4-oxazepane-2-carboxamide,
(2S)-N-{(15)-1-Cyano-2-[4-(4-methyl-3-oxo-3,4-dihydro-2H-1,4-benzothiazin-6-yl)phenyl]ethyl}-1,4-oxazepane-2-carboxamide,
(2S)-N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide,
(2S)-N-[(15)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide,
(2S)-N-[(15)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide,
(2S)-N-[(15)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide,
(2S)-N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide,
(2S)-N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide,
(2S)-N-{(1S)-1-Cyano-2-[4-(3-ethyl-2-oxo-2,3-dihydro-1,3-benzoxazol-5-yl)phenyl]ethyl}-1,4-oxazepane-2-carboxamide,
(2S)-N-[(15)-1-Cyano-2-{4-[3-(cyclopropylmethyl)-2-oxo-2,3-dihydro-1,3-benzoxazol-5-yl]phenyl}ethyl]-1,4-oxazepane-2-carboxamide,
(2S)-N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide,
(2S)-N-[(15)-1-Cyano-2-{4-[2-oxo-3-(propan-2-yl)-2,3-dihydro-1,3-benzoxazol-5-yl]phenyl}ethyl]-1,4-oxazepane-2-carboxamide,
(2S)-N-{(1S)-1-Cyano-2-[4-(4-methyl-3-oxo-3,4-dihydro-2H-1,4-benzoxazin-6-yl)phenyl]ethyl}-1,4-oxazepane-2-carboxamide,
(2S)-N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide,
(2S)-N-{(1S)-1-Cyano-2-[4-(5-cyanothiophen-2-yl)phenyl]ethyl}-1,4-oxazepane-2-carboxamide,
(2S)-N-[(1S)-2-(4′-Carbamoyl-3′-fluorobiphenyl-4-yl)-1-cyanoethyl]-1,4-oxazepane-2-carboxamide,
(2S)-N-{(15)-1-Cyano-2-[4-(1-methyl-2-oxo-1,2-dihydroquinolin-7-yl)phenyl]ethyl}-1,4-oxazepane-2-carboxamide,
(2S)-N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide,
(2S)-N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide,
(2S)-N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide,
(2S)-N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide,
(2S)-N-{(1S)-1-Cyano-2-[4-(3-methyl-2-oxo-2,3-dihydro-1,3-benzothiazol-5-yl)phenyl]ethyl}-1,4-oxazepane-2-carboxamide,
(2S)-N-{(1S)-1-Cyano-2-[4′-(methylsulfonyl)biphenyl-4-yl]ethyl}-1,4-oxazepane-2-carboxamide,
(2S)-N-{(1S)-2-[4′-(Azetidin-1-ylsulfonyl)biphenyl-4-yl]-1-cyanoethyl}-1,4-oxazepane-2-carboxamide,
(2S)-N-[(1S)-1-Cyano-2-(4′-fluorobiphenyl-4-yl)ethyl]-1,4-oxazepane-2-carboxamide,
(2S)-N-{(1S)-2-[4-(1,3-Benzothiazol-5-yl)phenyl]-1-cyanoethyl}-1,4-oxazepane-2-carboxamide, or
(2S)-N-[(1S)-1-Cyano-2-(4′-cyanobiphenyl-4-yl)ethyl]-1,4-oxazepane-2-carboxamide, or a pharmaceutically acceptable salt of one of the foregoing compounds.

It is also to be understood that certain compounds of Formula (I) may exist in solvated form, e.g., hydrates, including solvates of a pharmaceutically acceptable salt of a compound of Formula (I).

In one embodiment, the compound of Formula (I) provided in the methods described herein is a hydrate. In a further embodiment, the compound is a hydrate of brensocatib.

In one embodiment, a compound of Formula (I) is a racemate, a racemic mixture, a single enantiomer, an individual diastereomer or a diastereomeric mixture. It is to be understood that the present disclosure encompasses all such isomeric forms, e.g., the S,S diastereomer, the S,R diastereomer, the R,S diastereomer, and the R,R diastereomer disclosed herein, as well as a mixture of any two or more of the foregoing diastereomers.

Accordingly, in one embodiment, the compound of Formula (I) is (2S)-N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., brensocatib), shown below,

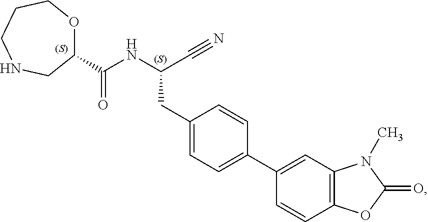

or a pharmaceutically acceptable salt thereof. In one embodiment, brensocatib is in the form of a hydrate.

In one embodiment, the compound of Formula (I) is (2R)-N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the R,R isomer), shown below,

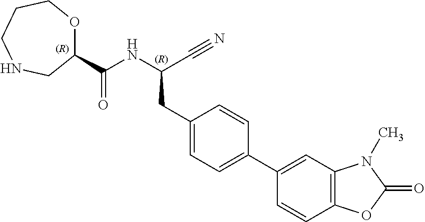

or a pharmaceutically acceptable salt thereof,

In one embodiment, the compound of Formula (I) is (2S)-N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the S,R isomer), shown below,

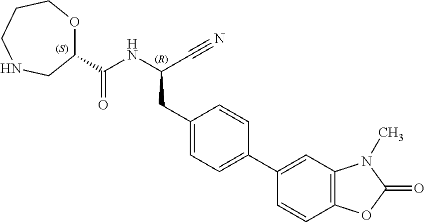

or a pharmaceutically acceptable salt thereof.

In one embodiment, the compound of Formula (I) is (2R)-N-{(1S)-1-Cyano-2-[4-(3-methyl-2-oxo-2,3-dihydro-1,3-benzoxazol-5-yl)phenyl]ethyl}-1,4-oxazepane-2-carboxamide (the R,S isomer), shown below,

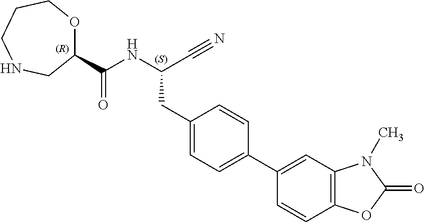

or a pharmaceutically acceptable salt thereof.

In one embodiment, the composition comprises a mixture of one or more of the aforementioned stereoisomers. The mixture in one embodiment, comprises a mixture of the S,S isomer (brensocatib) and the S,R isomer of a compound of Formula (I). In another embodiment, the composition comprises a mixture of the S,S isomer (brensocatib) and the R,S isomer. In yet another embodiment, the composition comprises a mixture of the S,S isomer (brensocatib) and the R,R isomer.

Certain compounds of Formula (I) may also contain linkages (e.g., carbon-carbon bonds, carbon-nitrogen bonds such as amide bonds) wherein bond rotation is restricted about that particular linkage, e.g., restriction resulting from the presence of a ring bond or double bond. Accordingly, it is to be understood that the present disclosure encompasses all such isomers. Certain compound of Formula (I) may also contain multiple tautomeric forms. It is to be understood that the present disclosure encompasses all such tautomeric forms. Stereoisomers may be separated using conventional techniques, e.g., chromatography or fractional crystallization, or the stereoisomers may be made by stereoselective synthesis.

In a further embodiment, the compounds of Formula (I) encompass any isotopically-labeled (or “radio-labelled”) derivatives of a compound of Formula (I). Such a derivative is a derivative of a compound of Formula (I) wherein one or more atoms are replaced by an atom having an atomic mass or mass number different from the atomic mass or mass number typically found in nature. Examples of radionuclides that may be incorporated include ²H (also written as “D” for deuterium). As such, in one embodiment, a compound of Formula (I) is provided where one or more hydrogen atoms are replaced by one or more deuterium atoms; and the deuterated compound is used in one of the methods provided herein.

In a further embodiment, a compound of Formula (I) is administered in the form of a prodrug which is broken down in the human or animal body to ultimately provide a compound of the Formula (I). Examples of prodrugs include in vivo hydrolysable esters of a compound of the Formula (I).

An in vivo hydrolysable (or cleavable) ester of a compound of Formula (I) that contains a carboxy or a hydroxy group is, for example, a pharmaceutically acceptable ester which is hydrolyzed in the human or animal body to produce the parent acid or alcohol. For examples of ester prodrugs derivatives, see, e.g., Beaumont et al. (2003). Curr. Drug. Metab. 4, 461-485, incorporated by reference herein in its entirety for all purposes.

Various other forms of prodrugs are known in the art, and can be used in the methods provided herein. For examples of prodrug derivatives, see, e.g., Rautio et al. (2008). Nature Reviews Drug Discovery, 7, 255-270, the disclosure of which is incorporated by reference herein in its entirety for all purposes.

The dosage of a compound of Formula (I) will vary with the compound employed, the mode of administration, the treatment desired and the type, symptoms or severity of CRS being treated. In some embodiments, the subject is administered a compound of Formula (I) at a daily dosage during the administration period of about 10 mg to about 100 mg, for example, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, including all values and subranges that lie therebetween. In some embodiments, the subject is administered a compound of Formula (I) at a daily dosage of about 10 mgs to about 65 mgs. In one embodiment, the subject is administered a compound of Formula (I) at a daily dosage of about 10 mg. In another embodiment, the subject is administered a compound of Formula (I) at a daily dosage of about 40 mg. In a further embodiment, the compound of Formula (I) is brensocatib.

In some embodiments, the daily dosage of a compound of Formula (I) during the administration period is in the range from 0.01 micrograms per kilogram body weight (g/kg) to 100 milligrams per kilogram body weight (mg/kg), for example, about 0.05 μg/kg, 0.1 μg/kg, 0.5 μg/kg, 1 μg/kg, 5 μg/kg, 10 μg/kg, 20 μg/kg, 30 μg/kg, 40 μg/kg, 50 μg/kg, 60 μg/kg, 70 g/kg, 80 μg/kg, 90 μg/kg, 100 μg/kg, 200 μg/kg, 300 μg/kg, 400 μg/kg, 500 μg/kg, 600 μg/kg, 700 μg/kg, 800 μg/kg, 900 μg/kg, 1 mg/kg, 20 mg/kg, 30 mg/kg, 40 mg/kg, 50 mg/kg, 60 mg/kg, 70 mg/kg, 80 mg/kg, 90 mg/kg, or 100 mg/kg, including all values and subranges that lie therebetween.

In one embodiment, the composition comprising the effective amount of a compound of Formula (I) is an oral dosage form. In a further embodiment, the compound of Formula (I) is administered as a 10 mg to 50 mg dosage form, for example, a 5 mg dosage form, a 10 mg dosage form, a 15 mg dosage form, a 20 mg dosage form, a 25 mg dosage form, a 30 mg dosage form, 35 mg dosage form, a 40 mg dosage form, a 45 mg dosage form or a 50 mg dosage form. In a further embodiment, the dosage form is a 10 mg, 25 mg or 40 mg dosage form. In a further embodiment, the dosage form is administered once daily. In a further embodiment, the compound is brensocatib, or a pharmaceutically acceptable salt thereof.

In some embodiments, the compound of Formula (I) is brensocatib and is present in the pharmaceutical composition in the range of about 1 mg to about 100 mg, for instance, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg about 75 mg, about 80 mg, about 85 mg, about 90 mg, about 95 mg, or about 100 mg, including all values and subranges that lie therebetween. In some embodiments, the amount of brensocatib in the pharmaceutical composition is in the range of about 10 mg to about 40 mg. In some embodiments, the amount of brensocatib in the pharmaceutical composition is in the range of about 25 mg to about 40 mg. In some embodiments, the amount of brensocatib in the pharmaceutical composition is in the range of about 10 mg to about 25 mg.

In one preferred embodiment, the compound of Formula (I) is brensocatib and is present in the pharmaceutical composition at about 10 mg. In a further embodiment, the pharmaceutical composition is administered to a subject once-daily during the administration period.

In another preferred embodiment, the compound of Formula (I) is brensocatib and is present in the pharmaceutical composition at about 25 mg. In a further embodiment, the pharmaceutical composition is administered to a subject once-daily during the administration period.

In another preferred embodiment, the compound of Formula (I) is brensocatib and is present in the pharmaceutical composition at about 40 mg. In a further embodiment, the pharmaceutical composition is administered to a subject once-daily during the administration period.

The methods provided herein comprise the administration of a composition comprising an effective amount of a compound of Formula (I), or a pharmaceutically acceptable salt thereof, to a patient in need of treatment or prophylaxis of CRS, during an administration period. The compounds of Formula (I) are inhibitors of dipeptidyl peptidase 1 (DPP1) activity. In one embodiment, the compound is brensocatib, or a pharmaceutically acceptable salt thereof. In a further embodiment, the compound is brensocatib.

In some embodiments, the subject undergoing one of the treatment methods provided herein exhibits one or more symptoms of CRS. In a further embodiment, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; or (o) any combinations thereof. In some embodiments, obstruction of the middle meatus is mucosal obstruction, edematous obstruction, or a combination thereof.

In some embodiments, the administration of the pharmaceutical composition reduces, diminishes the severity of, delays the onset of, or eliminates one or more symptoms of CRS. In a further embodiment, the one or more symptoms of CRS are: (a) nasal congestion; (b) nasal obstruction; (c) nasal discharge; (d) post-nasal drip; (e) facial pressure; (f) facial pain; (g) facial fullness; (h) reduced smell; (i) depression; (j) mucosal edema; (k) mucopurulent discharge; (1) obstruction of the middle meatus; (m) mucosal changes within the ostiomeatal complex and sinuses; (n) rhinorrhea; (o) or any combinations thereof. In some embodiments, the administration of the pharmaceutical composition enhances sinus drainage. In some embodiments, the one or more symptoms of CRS exhibited by the subject may be any symptoms described herein or known in the art to be associated with CRS. In some embodiments, the one or more symptoms of CRS are: nasal congestion, reduced smell, rhinorrhea, or any combination thereof. In some embodiments, the rhinorrhea is anterior rhinorrhea. In some embodiments, the rhinorrhea is posterior rhinorrhea.

In some embodiments of the method for treating CRS provided herein, a subject's symptoms, quality of life or other characteristics are assessed (i) prior to the administration period of the pharmaceutical composition, (ii) during the administration period of the pharmaceutical composition, (iii) subsequent to the administration period of the pharmaceutical composition, or a combination thereof. For example, in one embodiment, a subject's symptoms, quality of life or other characteristics are assessed (i) prior to the administration period of the pharmaceutical composition and (ii) during the administration period of the pharmaceutical composition. In another embodiment, a subject's symptoms, quality of life or other characteristics are assessed (i) prior to the administration period of the pharmaceutical composition and (ii) subsequent to the administration period of the pharmaceutical composition.

When assessing a subject's symptoms, quality of life or other characteristics prior to the administration period, the assessment can be made immediately prior to the administration period. “Immediately prior to the administration period”, as used herein, refers to from 0-24 hours prior to the initial administration of the pharmaceutical composition comprising a compound of Formula (I) to the subject. In one embodiment, when assessing a subject's symptoms, quality of life or other characteristics prior to the administration period, the assessment is carried out from 1 to 14 days prior to the administration period, from 1 to 13 days prior to the administration period, from 1 to 12 days prior to the administration period, from 1 to 11 days prior to the administration period, from 1 to 10 days prior to the administration period, from 1 to 9 days prior to the administration period, from 1 to 8 days prior to the administration period, from 1 to 7 days prior to the administration period, from 1 to 6 days prior to the administration period, from 1 to 5 days prior to the administration period, from 1 to 4 days prior to the administration period, from 1 to 3 days prior to the administration period, or from 1 to 2 days prior to the administration period. In yet another embodiment, when assessing a subject's symptoms, quality of life or other characteristics prior to the administration period, the assessment is carried out 1, 2, 3, 4, 5, 6, or 7 days prior to the administration period.

In one embodiment, the assessment of a subject's symptoms, quality of life or other characteristics is carried out two or more times, e.g., (i) two or more times prior to the administration period, (ii) two or more times during the administration period, and/or (iii) two or more times subsequent to the administration period. In these embodiments, the average output of the two or more assessments is used to make a determination of the subject's symptoms, quality of life or other characteristics. For example, the assessment can take place two or more consecutive days, e.g., 2 days, 3 days, 4 days, 5 days, 6 days, 7 days, 8 days, 9 days, or 10 consecutive days. In another embodiment, the assessment is carried out two or more times, and either every other day, or every third day.

In some embodiments, a method for treating CRS provided herein comprises reducing a composite severity score of two or more symptoms of CRS. As used herein, the “composite severity score” is a quantitative measure of the symptoms of CRS exhibited by the subject. The composite severity score, in one embodiment, is a sum total of all the daily symptoms exhibited by the subject. The composite severity score, in one embodiment, is a 0-9 point score based on symptoms for nasal congestion, anterior and/or posterior rhinorrhoea, and loss of smell. See, e.g., Bachert et al. (2019). Lancet 394, 1638-1650, particularly at p. 1641 right column and table 2. In some embodiments, the composite severity score is reduced during or subsequent to the administration period, as compared to the composite severity score measured prior to the administration period.

In some embodiments, the composite severity score of the subject during the administration period or subsequent to the administration period of the pharmaceutical composition is lower than the composite severity score of the subject prior to the administration period (e.g., immediately prior to the administration period). In some embodiments, the composite severity score of the subject during the administration period or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) lower than the composite severity score of the subject prior to the administration period, e.g., 1-day, 2-days, 3-days, 4-days, 5-days, or an average of multiple days, prior to the administration period. In some embodiments, the composite severity score of the subject during the administration period or subsequent to the administration period of the pharmaceutical composition is about 0.5, about 1, about 1.5, about 2, about 2.5, about 3, about 3.5, about 4, about 4.5, about 5, about 5.5, about 6, about 6.5, about 7, about 7.5, about 8, about 8.5, or about 9 points lower than the composite severity score of the subject prior to the administration period. In a further embodiment, the subject is a CRSsNP patient. In a further embodiment, the subject is a CRSsNP patient with an eosinophil count <300 cells/μL prior to the administration period. In another embodiment, the subject is a refractory CRS patient, e.g., a refractory CRSsNP patient.

In one embodiment, the composite severity score of the subject prior to the administration period or during/subsequent to the administration period is the subject's average composite severity score, taken over two or more consecutive days, for example, 2, 3, 4, 5, 6, 7, 8, 9, or 10 consecutive days, and the composite severity score is calculated as the average of these measurements, i.e., the average daily composite severity score. In one embodiment, to assess the composite severity score prior to the administration period or during/subsequent to the administration period, the composite severity score is assessed daily for two or more days, e.g., 2, 3, 4, 5, 6, 7, 8, 9, or 10, and the composite severity score is calculated as the average of these measurements in the corresponding periods, i.e., the average daily composite severity score prior to the administration period or during/subsequent to the administration period.

In one embodiment of a method for treating CRS, the treating comprises reducing a composite severity score of two or more symptoms of CRS, and the composite severity score is a sinus total symptom score (sTSS). In one embodiment, the sTSS reflects the signs and symptoms of patients with CRSsNP and is considered a well-defined and reliable patient-reported nasal symptom score. The sTSS, in one embodiment, is derived from the following items over the past 24 hours: nasal congestion, anterior and/or posterior rhinorrhea, and facial pain/pressure. The severity of each of the symptoms included in the sTSS, in one embodiment, is scored daily by the subject, e.g., in an electronic device. The assessment in one embodiment, is performed in the morning (AM). In one embodiment, prior to treatment (i.e., prior to the administration period), the subject has an sTSS of≥5.

In one embodiment, to assess the sTSS, the subjectuses a 0 to 3 categorical scale (where 0=no symptoms, 1=mild symptoms, 2=moderate symptoms and 3=severe symptoms) to assess the following symptoms over the previous 24 hours: (i) Congestion and/or obstruction; (ii) anterior/posterior rhinorrhea (runny nose); (iii) facial pain/pressure. See Fokkens et al., (2020). Rhinology 58(2), 82-111, incorporated herein by reference in its entirety.

In one embodiment, when assessing the sTSS, prior to the administration period, the subject self-assesses the sTSS daily for two or more days immediately prior to the administration period, for example, 2, 3, 4, 5, 6, 7, 8, 9, or 10 days immediately prior to the administration period, and the sTSS is calculated as the average of these measurements. In a further embodiment, when assessing the sTSS prior to the administration period, the subject self-assesses the sTSS daily for two or more consecutive days immediately prior to the administration period, for example, 2, 3, 4, 5, 6, 7, 8, 9, or 10 consecutive days immediately prior to the administration period, and the sTSS is calculated as the average of these measurements. In another embodiment, a single daily sTSS assessment is carried out, e.g., 1 day, 2 days, or 3 days immediately prior to the administration period, and the single score is used as the daily sTSS prior to the administration period. In one embodiment, to assess the sTSS during or subsequent to the administration period, the subject self-assesses the sTSS daily for two or more days, e.g., 2, 3, 4, 5, 6, 7, 8, 9, or 10 days, and the sTSS is calculated as the average of these measurements, i.e., the average daily sTSS during or subsequent to the administration period. In a further embodiment, to assess the sTSS during or subsequent to the administration period, the subject self-assesses the sTSS daily for two or more consecutive days, e.g., 2, 3, 4, 5, 6, 7, 8, 9, or 10 consecutive days, and the sTSS is calculated as the average of these measurements. In another embodiment, a single daily sTSS assessment is carried out and the single score is used as the daily sTSS score during or subsequent to the administration period.

In one embodiment, a method for treating CRS as disclosed herein comprises reducing a daily, or an average daily, sTSS of a CRSsNP subject during the administration period or subsequent to the administration period as compared to an sTSS of the subject prior to the administration period. In one embodiment, the subject is a CRSsNP patient with an eosinophil count of <300 cells/μL prior to the administration period. In another embodiment, the subject is a CRSsNP patient who has had prior nasal surgery. In another embodiment, the subject is a CRSsNP patient who has not had prior nasal surgery. In another embodiment, the subject is a CRSsNP patient who has an sTSS (covering nasal congestion, anterior/posterior rhinorrhea, facial pain/pressure) of≥5 prior to the administration period.

In some embodiments, a method for treating CRS as disclosed herein comprises decreasing the sTSS of the subject during the administration period or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the sTSS of the subject prior to the administration period. In some embodiments, the sTSS of the subject during the administration period or subsequent to the administration period is about 0.5, about 1, about 1.5, about 2, about 2.5, about 3, about 3.5, about 4, about 4.5, about 5, about 5.5, about 6, about 6.5, about 7, about 7.5, about 8, about 8.5, or about 9 points lower than the sTSS of the subject before administration of the pharmaceutical composition (i.e., prior to the administration period). In a further embodiment, the subject is a CRSsNP patient. In a further embodiment, the subject is a CRSsNP patient with an eosinophil count <300 cells/μL prior to the administration period. In another embodiment, the subject is a refractory CRS patient, e.g., a refractory CRSsNP patient.

In some embodiments, a method for treating CRS provided herein comprises decreasing a nasal congestion score (NCS) of the subject during the administration period or subsequent to the administration period as compared to an NCS of the subject prior to the administration period. In one embodiment, when assessing the NCS prior to the administration period, the subject self-assesses the NCS daily for two or more days, for example, 2, 3, 4, 5, 6, 7, 8, 9, or 10 days, and the NCS is calculated as the average of these measurements. In a further embodiment, when assessing the NCS prior to the administration period, the subject self-assesses the NCS daily for two or more consecutive days, for example, 2, 3, 4, 5, 6, 7, 8, 9, or 10 consecutive days, and the NCS is calculated as the average of these measurements. In another embodiment, a single daily NCS assessment is carried out and the single score is used as the daily NCS prior to the administration period. In one embodiment, to assess the NCS during or subsequent to the administration period, the subject self-assesses the NCS daily for two or more days, e.g., 3, 4, 5, 6, 7, 8, 9, or 10 days, and the NCS is calculated as the average of these measurements. In a further embodiment, the subject self-assesses the NCS daily for two or more consecutive days, e.g., 2, 3, 4, 5, 6, 7, 8, 9, or 10 consecutive days, and the NCS is calculated as the average of these measurements. In another embodiment, a single daily NCS assessment is carried out during or subsequent to the administration period and the single score is used as the daily NCS during or subsequent to the administration period.

In one embodiment, the NCS is a nasal congestion/obstruction severity score over a 24-hour period (i.e., a daily NCS). In a further embodiment, the NCS is part of a daily sinus total symptom score (sTSS) assessed by the subject. An NCS score is assessed in one embodiment with the following scale:
0 (=No symptoms);
1 (=Mild symptoms (symptoms clearly present, but minimal awareness and easily tolerated));
2 (=Moderate symptoms (definite awareness of symptoms that is bothersome but tolerable)); and
3 (=Severe symptoms (symptoms that are hard to tolerate, cause interference with activities or daily living)).

In one embodiment, the NCS is assessed as a daily NCS. In another embodiment, the NCS is assessed as an average daily NCS, e.g., over a period of 2 or more days, e.g., 2 or more consecutive days.

In one embodiment, the subject is a CRSsNP patient with a baseline NCS ≥2 (i.e., the subject's NCS prior to the administration period is ≥2). In some embodiments, a method provided herein comprises decreasing the NCS of the subject during the administration period or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the NCS of the subject prior to the administration period. In some embodiments, the NCS of the subject during the administration period or subsequent to the administration period of the pharmaceutical composition is about 0.5, about 1, about 1.5, about 2, about 2.5, or about 3 points lower than the NCS of the subject prior to the administration period. In a further embodiment, the subject is a CRSsNP patient. In a further embodiment, the subject is a CRSsNP patient with an eosinophil count <300 cells/μL prior to the administration period. In another embodiment, the subject is a refractory CRS patient, e.g., a refractory CRSsNP patient.

In some embodiments, the method of treating CRS provided herein comprises decreasing an anterior/posterior rhinorrhea severity score of the subject during or subsequent to the administration period as compared to an anterior/posterior rhinorrhea severity score of the subject prior to the administration period. In one embodiment, when assessing the anterior/posterior rhinorrhea severity score prior to the administration period, the subject self-assesses the anterior/posterior rhinorrhea severity score daily for 2 or more days, for example, 2, 3, 4, 5, 6, 7, 8, 9, or 10 days, and the anterior/posterior rhinorrhea severity score is calculated as the average of these measurements, i.e., the average daily anterior/posterior rhinorrhea severity score prior to the administration period. In a further embodiment, the average daily anterior/posterior rhinorrhea severity score is an average of scores taken over 2 or more consecutive days. In another embodiment, a single daily anterior/posterior rhinorrhea severity score assessment is carried out and the single score is used as the daily anterior/posterior rhinorrhea severity score prior to the administration period. In one embodiment, to assess the anterior/posterior rhinorrhea severity score during or subsequent to the administration period, the subject self-assesses the anterior/posterior rhinorrhea severity score daily for 2 or more days, e.g., 2, 3, 4, 5, 6, 7, 8, 9, or 10 days, and the anterior/posterior rhinorrhea severity score is calculated as the average of these measurements, i.e., the average daily anterior/posterior rhinorrhea severity score during or subsequent to the administration period. In a further embodiment, the average daily anterior/posterior rhinorrhea severity score is an average of scores taken over 2 or more consecutive days. In another embodiment, a single daily anterior/posterior rhinorrhea severity score assessment is carried out and the single score is used as the daily anterior/posterior rhinorrhea severity score during or subsequent to the administration period.

In one embodiment, the anterior/posterior rhinorrhea severity score assessed is a daily anterior/posterior rhinorrhea severity score. In another embodiment, the anterior/posterior rhinorrhea severity score assessed is an average daily anterior/posterior rhinorrhea severity score. In one embodiment, the subject is a CRSsNP patient.

An anterior/posterior rhinorrhea severity score, in one embodiment, is a component of a sTSS score. In one embodiment, regardless of whether the anterior/posterior rhinorrhea severity score is a component of a sTSS score, the former is assessed as follows:
0 (=No symptoms);
1 (=Mild symptoms (symptoms clearly present, but minimal awareness and easily tolerated));
2 (=Moderate symptoms (definite awareness of symptoms that is bothersome but tolerable)); and
3 (=Severe symptoms (symptoms that are hard to tolerate, cause interference with activities or daily living)).

In some embodiments, the methods provided herein comprise decreasing the anterior/posterior rhinorrhea severity score of the subject during the administration period or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the anterior/posterior rhinorrhea severity score of the subject prior to the administration period. In some embodiments, the anterior/posterior rhinorrhea severity score of the subject during the administration period or subsequent to the administration period is about 0.5, about 1, about 1.5, about 2, about 2.5, or about 3 points lower than the anterior/posterior rhinorrhea severity score of the subject prior to the administration period. In a further embodiment, the subject is a CRSsNP patient. In a further embodiment, the subject is a CRSsNP patient with an eosinophil count <300 cells/μL prior to the administration period. In another embodiment, the subject is a refractory CRS patient, e.g., a refractory CRSsNP patient.

In some embodiments, the method of treating CRS provided herein comprises decreasing a facial pain/pressure severity score of the subject during or subsequent to the administration period as compared to a facial pain/pressure severity score of the subject prior to the administration period. In one embodiment, when assessing the facial pain/pressure severity score prior to the administration period, the subject self-assesses the facial pain/pressure severity score daily for two or more days, for example, 2, 3, 4, 5, 6, 7, 8, 9, or 10 days, and the facial pain/pressure severity score is calculated as the average of these measurements, i.e., the average daily facial pain/pressure severity score prior to the administration period. In a further embodiment, the average daily facial pain/pressure severity score is an average of scores taken over 2 or more consecutive days. In another embodiment, a single daily facial pain/pressure severity score assessment is carried out and the single score is used as the daily facial pain/pressure severity score prior to the administration period. In one embodiment, to assess the facial pain/pressure severity score during or subsequent to the administration period, the subject self-assesses the facial pain/pressure severity score daily for two or more days, e.g., 2, 3, 4, 5, 6, 7, 8, 9, or 10 days, and the facial pain/pressure severity score is calculated as the average of these measurements, i.e., the average daily facial pain/pressure severity score during or subsequent to the administration period. In a further embodiment, the average daily facial pain/pressure severity score is an average of scores taken over 2 or more consecutive days. In another embodiment, a single daily facial pain/pressure severity score assessment is carried out and the single score is used as the daily facial pain/pressure severity score during or subsequent to the administration period. In one embodiment, the facial pain/pressure severity score assessed is a daily facial pain/pressure severity score, i.e., one assessed based on the facial pain/pressure severity over the past 24 hours. In another embodiment, the facial pain/pressure severity score assessed is an average daily facial pain/pressure severity score. In one embodiment, the subject is a CRSsNP patient.

Facial pain/pressure severity score, in one embodiment, is part of an sTSS score. In one embodiment, the facial pain/pressure severity score is assessed as follows:
0 (=No symptoms);
1 (=Mild symptoms (symptoms clearly present, but minimal awareness and easily tolerated));
2 (=Moderate symptoms (definite awareness of symptoms that is bothersome but tolerable)); and
3 (=Severe symptoms (symptoms that are hard to tolerate, cause interference with activities or daily living)).

In some embodiments, the methods provided herein comprise decreasing the facial pain/pressure severity score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the facial pain/pressure severity score of the subject prior to the administration period. In some embodiments, the facial pain/pressure severity score of the subject during or subsequent to the administration period is about 0.5, about 1, about 1.5, about 2, about 2.5, or about 3 points lower than the facial pain/pressure severity score of the subject prior to the administration period. In a further embodiment, the subject is a CRSsNP patient. In a further embodiment, the subject is a CRSsNP patient with an eosinophil count <300 cells/μL prior to the administration period. In another embodiment, the subject is a refractory CRS patient, e.g., a refractory CRSsNP patient.

In some embodiments, the method of treating CRS provided herein comprises decreasing a Lund-Mackay (LMK) score of the subject during or subsequent to the administration period as compared to the Lund-Mackay score of the subject prior to the administration period. In a further embodiment, the subject is a CRSsNP patient. In a further embodiment, the subject is a refractory CRSsNP patient.

The LMK total score is based on an assessment of a computerized tomography (CT) scan findings for each sinus area of a subject. The extent of opacification is rated between 0 (normal), 1 (partial opacification), to 2 (total opacification). These points can then be applied to the maxillary, anterior ethmoid, posterior ethmoid, sphenoid, and frontal sinus on each side. The osteomeatal complex can also be graded in a LMK score as 0 (not occluded) or 2 (occluded). Therefore, a maximum score of 12 per side is then derived for a total score of 24 (Lund and Mackay (1993). Rhinology 31, 183-184, incorporated herein by reference in its entirety).

In some embodiments, the CT scan is of the paranasal sinuses of the subject, one or both of ostiomeatal complexes of the subject, or a combination thereof. In some embodiments, the CT scan is of the right ostiomeatal complex of the subject, the left ostiomeatal complex of the subject, or a combination thereof. In some embodiments, the CT scan is of one or more of (a) right frontal sinuses; (b) left frontal sinuses; (c) right anterior ethmoidal sinuses; (d) left anterior ethmoidal sinuses; (e) right posterior ethmoidal sinuses; (f) left posterior ethmoidal sinuses; (g) right maxillary sinuses; (h) left maxillary sinuses; (i) right sphenoid sinuses of the subject (j) left sphenoid sinuses of the subject; or (k) combinations thereof.

In some embodiments, the LMK score of the subject prior to the administration period is greater than that of a subject who does not have CRS. In some embodiments, the Lund-Mackay score of the subject prior to the administration period of the pharmaceutical composition is greater than or equal to 4. For example, in some embodiments, the Lund-Mackay score of the subject in need of treatment, prior to the administration period of the pharmaceutical composition is 4, 5, 6, 7, 8, 9, 10, 11, 12, 13, 14, 15, 16, 17, 18, 19, 20, 21, 22, 23, or 24. In a further embodiment, the subject is a CRSsNP patient.

In some embodiments, a method for treating CRS provided herein comprises decreasing the LMK score of the subject during or subsequent to the administration period as compared to the Lund-Mackay score of the subject prior to the administration period. In a further embodiment, treating comprises decreasing the LMK score to <4. In some embodiments, treating comprises decreasing the LMK score of the subject during or subsequent to the administration period to 0, 1, 2 or 3. In some embodiments, the methods provided herein comprise decreasing the LMK score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the LMK score of the subject prior to the administration period, e.g., 1, 2, 3, 4, 5, 6 or 7 days prior to the administration period. In some embodiments, the methods provided herein comprise decreasing the LMK score of the subject during or subsequent to the administration period by 1, 2, 3, 4, 5, 6, 7, 8, 9, 10, 11, 12, 13, 14, 1,5 16, 17, 18, 19, 20, 21, 22, 23, or 24. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

CT scanning is useful for assessing changes in paranasal sinus mucosa. Mucosal thickening on CT currently is considered an objective diagnostic criterion for CRS. A 3-dimensional assessment that determines the percent volume of disease from CT images is the most comprehensive and precise information that could be obtained about disease severity on CT images. In some embodiments, a method of treating CRS provided herein comprises decreasing the percentage of sinus opacification of the subject as measured by CT scan volumetry during or subsequent to the administration period as compared to the percentage of sinus opacification of the subject prior to the administration period. In a further embodiment, the subject is a CRSsNP patient. In a further embodiment, the subject is a CRSsNP patient with an eosinophil count <300 cells/μL prior to the administration period, e.g., 1, 2, 3, 4, 5, 6 or 7 days prior to the administration period. In one embodiment, the subject is a refractory CRSsNP patient.

In some embodiments, one or more symptoms of CRS are assessed in the subject by a rhinoscopy. The rhinoscopy of the subject, in one embodiment, can reveal one or more of: (i) nasal dryness, (ii) dried nasal mucus, (iii) fibrin deposition, (iv) nasal obstruction, or (v) any combination thereof. In some embodiments, a method of treating CRS provided herein comprises decreasing a rhinoscopy sum score of the subject during or subsequent to the administration period, as compared to the rhinoscopy sum score of the subject prior to the administration period, e.g., 1, 2, 3, 4, 5, 6 or 7 days prior to the administration period. As used herein, the “rhinoscopy sum score” refers to the sum score that is attained from objectively recorded, clinical endoscopy findings based on: (i) nasal dryness, (ii) dried nasal mucus, (iii) fibrin deposition, and (iv) nasal obstruction. All variables pertaining to rhinoscopic mucosal findings are evaluated using a 4-point scale as follows: absent=0, mild=1, moderate=2, and severe=3. Further details of the rhinoscopy sum score are provided in Gouteva et al. (2014). J Allergy (Cairo). 2014, 635490, the contents of which are incorporated herein by reference in its entirety. In some embodiments, the methods provided herein include performing a rhinoscopy on the subject, wherein the rhinoscopy is performed both (i) prior to the administration period, and (ii) during or subsequent to the administration period.

In some embodiments, the rhinoscopy sum score of the subject prior to the administration period is greater than that of a control subject who does not have CRS. In some embodiments, the rhinoscopy sum score of the subject prior to the administration period, e.g., 1, 2, 3, 4, 5, 6 or 7 days prior to the administration period, is greater than 1. For example, in some embodiments, the rhinoscopy sum score of the subject prior to the administration period is 2 or 3, e.g., 1, 2, 3, 4, 5, 6 or 7 days prior to the administration period.

In some embodiments, the rhinoscopy sum score of the subject during or subsequent to the administration period is ≤1. In a further embodiment, the rhinoscopy sum score of the subject during or subsequent to the administration period is 0 or 1. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

In some embodiments, the CRS treatment method provided herein comprises decreasing the Sino-Nasal Outcome Test-22 (SNOT-22) score of the subject during or subsequent to the administration period, as compared to the SNOT-22 score of the subject prior to the administration period, e.g., 1, 2, 3, 4, 5, 6 or 7 days prior to the administration period. The “SNOT-22” is a patient-reported measure of outcome developed for use in CRS with or without nasal polyps and contains 22 individual questions on a 5-category scale. The questions cover a broad range of health and health-related quality of life problems including physical problems, functional limitations, and emotional consequences. The range of the SNOT-22 score is O to 110 with the minimal clinically important difference (MCID) of ≥8.9 points. Lower scores indicate less impact and a better quality of life. The recall period for subject's reporting a SNOT-22 score is the previous 2 weeks. The 22 individual questions in the SNOT-22 are grouped into five (5) domains and include the nasal, ear, sleep, general/practical, and emotional domains. Further details of SNOT-22 are provided in Hopkins, et al. (2009). Clin. Otolaryngol. 34, 447-454, and Kennedy et al. (2013). Ann Allergy Asthma Immunol. 111(4), 246-251, the contents of each of which are incorporated herein by reference in their entireties.

In some embodiments, the method for treating CRS comprises reducing the subject's SNOT-22 score during or subsequent to the administration period by 8 points or more, 9 points or more, or 10 points or more, compared to the SNOT-22 score of the subject prior to the administration period. The SNOT-22 score, in one embodiment, is measured immediately prior to undergoing the treatment method. In another embodiment, the SNOT-22 score is measured 1, 2, 3, 4, 5, 6 or 7 days prior to the administration period.

In some embodiments, the method for treating CRS comprises reducing the subject's SNOT-22 score during or subsequent to the administration period by about 8 to about 40 points, by about 8 to about 30 points, by about 8 to about 20 points, by about 8 to about 18 points, by about 8 to about 16 point or by about 8 to about 14 points, compared to the SNOT-22 score of the subject prior to the administration period. In some embodiments, the method for treating CRS comprises reducing the subject's SNOT-22 score during or subsequent to the administration period by about 9 to about 40 points, by about 9 to about 30 points, by about 9 to about 20 points, by about 9 to about 18 points, by about 9 to about 16 point or by about 9 to about 14 points, compared to the SNOT-22 score of the subject prior to the administration period.

In some embodiments, the SNOT-22 score of the subject prior to the administration period is greater than that of a control subject who does not have CRS. In some embodiments, the SNOT-22 score of the subject prior to the administration period, is greater than or equal to 30. For example, in some embodiments, the SNOT-22 score of the subject prior to the administration period is from about 30 to about 110, from about 30 to about 100, from about 30 to about 90, from about 30 to about 80, from about 30 to about 70, from about 30 to about 60, or from about 30 to about 50. In another embodiment, the SNOT-22 score of the subject is from about 40 to about 110, from about 40 to about 100, from about 40 to about 90, from about 40 to about 80, from about 40 to about 70, or from about 40 to about 60. In even another embodiment, the SNOT-22 score of the subject prior to the administration period is from about 50 to about 110, from about 60 to about 110, from about 70 to about 110, from about 80 to about 110, or from about 90 to about 110.

In some embodiments, the SNOT-22 score of the subject prior to the administration period is greater than that of a control subject who does not have CRS. In some embodiments, the SNOT-22 score of the subject prior to the administration period is ≥30. For example, in some embodiments, the SNOT-22 score of the subject prior to the administration period is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110. In some embodiments, the SNOT-22 score of the subject prior to the administration period is ≥20.

In some embodiments, a method for treating CRS provided herein comprises decreasing the SNOT-22 score of the subject during or subsequent to the administration period by at least about 2% (e.g.,, at least about 3%, at least about 4%, at least about 5%, at least about 10%, at least about 15%, about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the SNOT-22 score of the subject prior to the administration period. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

In some embodiments, a method for treating CRS provided herein comprises decreasing the SNOT-22 score of the subject during or subsequent to the administration period by at least about 10%. In some embodiments, the SNOT-22 score of the subject during or subsequent to the administration period is <30. For example, in some embodiments, the SNOT-22 score of the subject during or subsequent to the administration period is 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0. In some embodiments, the SNOT-22 score of the subject during or subsequent to the administration period is <20. In one embodiment where treating CRS comprises decreasing the subject's SNOT-22 score, the subject is a CRSsNP patient. In a further embodiment, the subject is a CRSsNP patient with an eosinophil count <300 cells/μL prior to the administration period. In one embodiment, the subject is a refractory CRSsNP patient.

In some embodiments, a method of treating CRS provided herein comprises decreasing a Visual Analog Scale (VAS) score of the subject during or subsequent to the administration period as compared to a VAS score of the subject prior to the administration period. In one embodiment, the subject has a VAS score of ≥5 prior to the administration period, e.g., immediately prior to the treatment method or 1, 2, 3, 4, 5, 6, or 7 days prior to the administration period. In another embodiment, the subject is a CRSsNP patient. In one embodiment, the subject is a refractory CRSsNP patient.

The VAS is validated for use in adults with CRS to evaluate total severity of the disease. See Fokkens et al., (2012). Rhinol Suppl.; 50(23), 1-298, incorporated herein by reference in its entirety. Participants are asked to answer the question “How troublesome are your symptoms of rhinosinusitis?” The VAS ranks from 0 (not troublesome) to 10 (worst thinkable troublesome). The VAS scale can be used to determine disease severity (mild, moderate, or severe). A VAS score of>5 is considered to affect quality of life (i.e., uncontrolled symptoms), a VAS score of>2 to ≤5 is considered partially controlled symptoms, and <2 is considered well controlled symptoms. See Mullol et al. (2022). J Allergy Clin Immunol Pract. 10(6), 1434-1453, incorporated herein by reference in its entirety. Based on the total severity, VAS score is as follows:
Mild=VAS 0-3
Moderate=VAS >3-7
Severe=VAS >7-10

The VAS score, in one embodiment, is used to assess a subject's symptoms over the past 24 hours. In a further embodiment, the VAS is self-administered by the subject. In a further embodiment, the VAS is self-administered electronically.

In some embodiments, the method for treating CRS provided herein comprises decreasing the VAS score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the VAS score of the subject prior to the administration period, e.g., immediately prior to the treatment method or 1, 2, 3, 4, 5, 6, or 7 days prior to the administration period. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

In one embodiment, a method for treating CRS comprises reducing the subject's VAS score from a severe score to a moderate score. In another embodiment, a method for treating CRS comprises reducing the subject's VAS score from a severe score to a mild score. In even another embodiment, a method for treating CRS comprises reducing the subject's VAS score from a moderates score to a mild score.

In one embodiment, prior to the administration period, the subject's VAS score is ≥5.

In some embodiments, the methods provided herein comprise increasing a Peak Nasal Inspiratory Flow (PNIF) of the subject during or subsequent to the administration period as compared to a PNIF of the subject prior to the administration period. The PNIF is an assessment of nasal passage obstruction by measuring air flow through both nasal cavities during forced inspiration expressed in liters per minute. The PNIF is a well validated technique for the evaluation of nasal flow through the nose (Scadding et al. (2011). Clin Transl Allergy 1:2, incorporated herein by reference in its entirety). The PNIF in one embodiment, is measured using an in-check portable nasal inspiratory flow meter. In a further embodiment, the subject is a CRSsNP patient. In one embodiment, the subject is a refractory CRSsNP patient.

In one embodiment, three PNIF readings within a day are obtained, and the highest reading is used for evaluation. In some embodiments, the methods provided herein comprise increasing a PNIF of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to a PNIF of the subject prior to the administration period. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

In some embodiments, a method provided herein comprises improving a Patient Global Impression of Severity (PGI-S) score or a Patient Global Impression of Change (PGI-C) score of the subject during or subsequent to the administration period as compared to the respective score of the subject prior to the administration period. In a further embodiment, the subject is a CRSsNP patient. In a further embodiment, the subject is a refractory CRSsNP patient. The PGI-S and PGI-C are both 1-item questionnaires using balanced Likert scales that ask the participant to rate the severity of CRS. Specifically, the PGI-S uses a single state 5-point categorical scale with a higher score indicating more severe CRS. The PGI-C is used to rate at a particular time point the perceived change in CRS status in response to treatment via a transitional 7-point categorical scale. In the case of the PGI-C, a decreased score indicates improvement in CRS and an increased score indicates worsening in CRS.

In some embodiments, the methods provided herein comprise improving a PGI-S score or a PGI-C score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to a PGI-S score or a PGI-C score of the subject prior to the administration period. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient

In some embodiments, the methods provided herein comprise improving a PGI-S score of the subject during or subsequent to the administration period by about 0.5, about 1, about 1.5, about 2, about 2.5, about 3, about 3.5, about 4, or about 4.5 points, as compared to a PGI-S score of the subject prior to the administration period. In one embodiment, the methods provided herein comprise improving a PGI-S score of the subject during or subsequent to the administration period by about 0.5 to about 4.5 points, about 1 to about 4.5 points, about 1.5 to about 4.5 points, about 2 to about 4.5 points, about 2.5 to about 4.5 points, about 3 to about 4.5 points, about 0.5 to about 4 points, about 0.5 to about 3.5 points, about 0.5 to about 3 points, about 0.5 to about 2.5 points, about 0.5 to about 2 points, about 1 to about 4 points, about 1 to about 3.5 points, about 1.5 to about 3 points, about 1.5 to about 2.5 points, about 1.5 to about 2 points, as compared to a PGI-S score of the subject prior to the administration period. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

In some embodiments, the methods provided herein comprise improving a PGI-C score of the subject during the administration period or subsequent to the administration period by about 0.5, about 1, about 1.5, about 2, about 2.5, about 3, about 3.5, about 4, about 4.5 points, about 5, about 5.5, about 6, or about 6.5 points, as compared to a PGI-C score of the subject prior to the administration period. In one embodiment, the methods provided herein comprise improving a PGI-C score of the subject during or subsequent to the administration period by about 0.5 to about 6.5 points, about 1 to about 6.5 points, about 1.5 to about 6.5 points, about 2 to about 6.5 points, about 2.5 to about 6.5 points, about 3 to about 6.5 points, about 3.5 to about 6.5 points, about 4 to about 6.5 points, about 4.5 to about 6.5 points, about 5 to about 6.5 points, about 0.5 to about 6 points, about 0.5 to about 5.5 points, about 0.5 to about 5 points, about 0.5 to about 4.5 points, about 0.5 to about 4 points, about 1 to about 6.5 points, about 1.5 to about 6 points, about 2 to about 5.5 points, about 2.5 to about 5 points, about 3 to about 4.5 points, as compared to a PGI-C score of the subject prior to the administration period. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

In some embodiments, the methods provided herein comprise increasing the length of time to first use of rescue with a systemic corticosteroid, an antibiotic, or nasal surgery of the subject, or as compared to a control subject, wherein the control subject has CRS and is not administered the pharmaceutical composition. In a further embodiment, the subject and control subject are each a CRSsNP patient, e.g., a refractory CRSsNP patient. In one embodiment, the length of time to first use of rescue is increased by about 1 day, about 3 days, about 1 week, about 2 weeks, about 3 weeks, about 4 weeks, about 5 weeks, or about 6 weeks. In another embodiment, the length of time to first use of rescue is increased by at least about 1 day, at least about 3 days, at least about 1 week, at least about 2 weeks, at least about 3 weeks, at least about 4 weeks, at least about 5 weeks, or at least about 6 weeks. In another embodiment, the length of time to first use of rescue is increased about 20 days to about 100 days, about 30 days to about 100 days, about 20 days to about 75 days, about 20 days to about 50 days, or about 20 days to about 40 days. In another embodiment, the length of time to first use of rescue is increased at least 1 month, e.g., about 1 month to about 6 months, about 1 month to about 4 months, or about 1 month to about 3 months.

In some embodiments, a method of treating CRS provided herein comprises reducing the frequency of rescue with a systemic corticosteroid, an antibiotic, or nasal surgery of the subject due to worsening of CRS symptoms, or as compared to a control subject, wherein the control subject has CRS and is not administered the pharmaceutical composition. In a further embodiment, the subject and control subject are each a CRSsNP patient, e.g., a refractory CRSsNP patient. In one embodiment, the frequency of rescue is calculated over a period of about 1 week, about 1 month, about 2 months, about 3 months, about 4 months, about 5 months, about 6 months, about 9 months, about 12 months, about 15 months, about 18 months, about 21 months, or about 24 months. In one embodiment, the frequency of rescue of the subject is reduced by about 5%, about 10%, about 15%, about 20%, about 25%, about 30%, about 35%, about 40%, or about 50%. In another embodiment, the frequency of rescue of the subject is reduced by at least about 5%, at least about 10%, at least about 15%, at least about 20%, at least about 25%, at least about 30%, at least about 35%, at least about 40%, or at least about 50%.

In some embodiments, the methods comprise increasing the University of Pennsylvania Smell Identification Test (UPSIT) score of the subject during or subsequent to the administration period, as compared to the UPSIT score of the subject prior to the administration period, e.g., immediately prior to the treatment method or 1, 2, 3, 4, 5, 6, or 7 days prior to the administration period. The “UPSIT” is a commercially available test to evaluate the smell identification function of the individual's olfactory system. The theoretical range of an UPSIT score falls in the range of 0 through 40. Further details are provided at Doty et al. (1989). Perception & Psychophysics 45, 381-384 and Saltagi et al. (2021). Allergy & Rhinology, 12, 1-17, the contents of each of which are incorporated herein by reference in their entirety.

In some embodiments, the method of treating CRS comprises increasing the UPSIT score of the subject during or subsequent to the administration period compared to the UPSIT score of the subject prior the administration period. In some embodiments, the UPSIT score of the subject prior to the administration period is less than 33. In one embodiment, the UPSIT score of the subject prior to the administration period is less than 18. In some embodiments, the UPSIT score of the subject prior to the administration period is 32, 31, 30, 29, 28, 27, 26, 25, 24, 23, 22, 21, 20, 19, 18, 17, 16, 15, 14, 13, 12, 11, 10, 9, 8,7, 6, 5,4, 3,2, 1, or 0.

In some embodiments, the UPSIT score of the subject during or subsequent to the administration period is greater than the UPSIT score of the subject prior to the administration period. In some embodiments, the UPSIT score of the subject during or subsequent to the administration period is >33. In some embodiments, the UPSIT score of the subject during or subsequent to the administration period is >18. In some embodiments, the UPSIT score of the subject during or subsequent to the administration period is 19, 20, 21, 22, 23, 24, 25, 26, 27, 28, 29, 30, 31, 32, 33, 34, 35, 36, 37, 38, 39, or 40. In some embodiments, the methods provided herein comprise increasing the UPSIT score of the subject during or subsequent to the administration period by 1, 2, 3, 4, 5, 6, 7, 8, 9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, or 40. In another embodiment, a method provided herein comprises increasing the UPSIT score of the subject during or subsequent to the administration period by from about 1 to about 40, from about 1 to about 30, from about 1 to about 20, from about 2 to about 30, from about 2 to about 20, from about 5 to about 40, from about 5 to about 30, from about 5 to about 20, or from about 5 to about 10.

In some embodiments, the method comprises increasing the UPSIT score of the subject during or subsequent to the administration period by at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) as compared to the UPSIT score of the subject prior to the administration period, e.g., immediately prior to the treatment method or 1, 2, 3, 4, 5, 6, or 7 days prior to the administration period. In a further embodiment, the subject is a CRSsNP patient. In a further embodiment, the subject is a CRSsNP patient with an eosinophil count <300 cells/μL prior to the administration period. In another embodiment, the subject is a refractory CRSsNP patient.

In some embodiments, the method comprises increasing the UPSIT score of the subject during or subsequent to the administration period by at least about 2 fold (for example, about 3 fold, about 4 fold, about 5 fold, about 10 fold, about 15 fold, about 20 fold, about 25 fold, about 30 fold, about 35 fold, or about 40 fold, including all values and subranges therebetween) as compared to the UPSIT score of the subject prior to the administration period, e.g., immediately prior to the treatment method or 1, 2, 3, 4, 5, 6, or 7 days prior to the administration period. In a further embodiment, the subject is a CRSsNP patient. In a further embodiment, the subject is a CRSsNP patient with an eosinophil count <300 cells/μL prior to the administration period. In another embodiment, the subject is a refractory CRSsNP patient.

In some embodiments, a method for treating CRS provided herein comprises determining a sinonasal endoscopy score of the subject, e.g., via a modified Lund-Kennedy (MLK) endoscopic scoring system. As used herein, the “MLK” endoscopic scoring system is as described in Psaltis et al. (2014) The Laryngoscope 124, 2216-2223, incorporated herein by reference in its entirety. The theoretical range of the MLK endoscopic scoring system is 0 through 12.

One embodiment of a method for treating CRS in a subject in need thereof comprises decreasing the MLK endoscopy score of the subject during or subsequent to the administration period, as compared to the subject's MLK endoscopy score prior to the administration period, e.g., immediately prior to the treatment method or 1, 2, 3, 4, 5, 6, or 7 days prior to the administration period. In some embodiments, the MLK score of the subject prior to the administration period is greater than or equal to 4. For example, in some embodiments, the MLK score of the subject prior to the administration period is 5, 6, 7, 8, 9, 10, 11, or 12. In another embodiment, the MLK score of the subject prior to the administration period is from about 5 to about 12, from about 6 to about 12, from about 7 to about 12, from about 8 to about 12 or from about 10 to about 12. In some embodiments, the MLK score of the subject prior to the administration period is higher than that of a control subject who does not have CRS.

In some embodiments, the MLK score of the sinonasal endoscopy of the subject during or subsequent to the administration period is <4. For example, in some embodiments, the MLK score of the sinonasal endoscopy of the subject during or subsequent to the administration period is 3, 2, 1 or 0. In some embodiments, the MLK score of the subject during or subsequent to the administration period is decreased by 1, 2, 3, 4, 5, 6, 7, 8, 9, 10, 11, or 12. In another embodiment, the MLK score of the subject during or subsequent to the administration period is decreased by from 1 to about 12, from about 2 to about 12, from about 3 to about 12, from about 4 to about 12, from about 5 to about 12, from about 6 to about 12, from about 7 to about 12, from about 8 to about 12 or from about 9 to about 12. In yet another embodiment, the MLK score of the subject during or subsequent to the administration period is decreased by from 1 to about 12, from about 2 to about 11, from about 3 to about 10, from about 4 to about 9.

In some embodiments, a method for treating CRS in a subject in need thereof comprises decreasing the MLK score of the subject during or subsequent to the administration period by at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, including all values and subranges that lie therebetween) as compared to the MLK score of the subject prior to the administration period. In a further embodiment, the subject is a CRSsNP patient. In a further embodiment, the subject is a CRSsNP patient with an eosinophil count <300 cells/μL prior to the administration period. In one embodiment, the subject is a refractory CRSsNP patient.

In some embodiments, the number of neutrophils in a biological sample obtained from the subject prior to the administration period is greater than the number of neutrophils in a biological sample obtained from a control subject who does not have CRS. In some embodiments, the number of neutrophils in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than the number of neutrophils in a biological sample obtained from a control subject who does not have CRS. In some embodiments, the number of neutrophils in a biological sample obtained from the subject prior to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900% or about 1000%, including all values and subranges that lie therebetween) greater than the number of neutrophils in a biological sample obtained from control subject who does not have CRS.

In some embodiments, the level of Intercellular Adhesion Molecule 1 (ICAM1) in a biological sample obtained from the subject prior to the administration period is greater as compared to a control subject who does not have CRS. In some embodiments, the level of ICAM1 in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) greater than the level of ICAM1 in a biological sample obtained from a control subject who does not have CRS. In some embodiments, the level of ICAM1 in a biological sample obtained from the subject prior to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about ⁸⁰⁰%, about 900% or about 1000%, including all values and subranges that lie therebetween) greater than the level of ICAM1 in a biological sample obtained from a control subject who does not have CRS.

In some embodiments, the expression of one or more genes is altered in a tissue of the subject prior to the administration period, as compared to the expression of the one or more genes in a tissue obtained from a control subject who does not have CRS. In some embodiments, the expression of the one or more genes is increased in a tissue of the subject prior to the administration period, as compared to a control subject who does not have CRS. In some embodiments, the expression of one or more genes in a tissue obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) higher compared to the expression of the one or more genes in a tissue obtained from a control subject who does not have CRS.

In some embodiments, the expression of one or more genes in a tissue obtained from the subject prior to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900% or about 1000%, including all values and subranges that lie therebetween) higher compared to the expression of the one or more genes in a tissue obtained from a control subject who does not have CRS.

In some embodiments, the expression of the one or more genes is decreased in a tissue of the subject prior to the administration period, as compared to a control subject who does not have CRS. In some embodiments, the expression of one or more genes in a tissue obtained from the subject prior to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, including all values and subranges that lie therebetween) less than the expression of the one or more genes in a tissue obtained from a control subject who does not have CRS.

In some embodiments, the tissue is sinonasal tissue. In some embodiments, the one or more genes encodes a protein selected from the group consisting of T-bet, GATA binding protein 3 (GATA-3), RAR-related orphan receptor C, IFN-7, interleukin (IL)-5, IL-17A, IL-22, IL-23, IL-8, TLR-2, and IL-10.

In some embodiments, the subject in need of treatment is at a risk for developing CRS. As such, where a subject in need of treatment is at a risk for developing CRS, a method provided herein can be considered prophylactic. In some embodiments, the subject at a risk for developing CRS has or had one or more of the following conditions: acute rhinosinusitis, viral respiratory tract infection, allergic rhinitis, nonallergic rhinitis, asthma, bronchitis, pneumonia, gastroesophageal reflux disease, adenotonsillitis, sleep apnea, otitis media, allergic or nonallergic upper airway disease, allergic or nonallergic lower airway disease, epithelial cell disorder, common variable immunodeficiency, HIV infection, cystic fibrosis (CF), ciliary dyskinesia, granulomatosis with polyangiitis, sarcoidosis, and chronic obstructive pulmonary disease.

In some embodiments, the subject in need of treatment is or was repeatedly exposed to tobacco smoke. As used herein, “repeated exposure” to tobacco smoke relates to exposure that is frequent enough to be associated with, result in, or increase the risk of developing one or more adverse effects of inhaling tobacco smoke. Non-limiting adverse effects of inhaling tobacco smoke are cancer (e.g., lung cancer), coronary heart disease, respiratory infections, stroke, lung disease, diabetes, chronic obstructive pulmonary disease (COPD), emphysema, chronic bronchitis, tuberculosis, eye diseases, immune dysfunction, and rheumatoid arthritis. In some embodiments, the subject is exposed tobacco smoke at least once a month, for example, at least once in two weeks, at least once a week, at least every alternate day, at least every day, or several times a day. In some embodiments, the subject is an active smoker of tobacco-containing products, such as cigarettes. In some embodiments, the subject is passively exposed to tobacco smoke.

In some embodiments, the subject in need of treatment has one or more mutations in a gene encoding a protein selected from the group consisting of: Ring1A and YY1 binding protein (RYBP), acyloxyacyl hydrolase (AOAH), IL-1 receptor-associated kinase 4, IL-1 receptor-like 1, Toll-like receptor (TLR)-2, TLR-1, TLR-5, cystic fibrosis transmembrane conductance regulator (CFTR), and transforming growth factor beta-1. In some embodiments, the one or more mutations is a single nucleotide polymorphism. In some embodiments, the single nucleotide polymorphism is rs4504543 in the gene encoding AOAH, or rs4532099 in the gene encoding RYBP. Further details on mutations and risk factors associated with CRS are provided in Cho et al., J Allergy Cin ImmunolPract. 2016, the contents of which are herein incorporated by reference in its entirety.

In some embodiments of a method of treating CRS provided herein, the CRS is associated with the presence or development of one or more conditions selected from the group consisting of allergic conjunctivitis, atopic dermatitis, asthma, urinary tract infections, and skin/soft tissue infections.

In one embodiment, the subject in need of treatment is an adult with CRS who has two or more symptoms, one of which is either nasal blockage, nasal obstruction, nasal congestion or nasal discharge (anterior/posterior nasal drip), (i) with or without facial pain and/or pressure; and (ii) with or without a reduction or loss of smell; for >12 weeks.

In one embodiment, the subject in need of treatment is a child with CRS who has two or more symptoms, one of which is either nasal blockage, nasal obstruction, nasal congestion, or nasal discharge (anterior/posterior nasal drip), (i) with or without facial pain and/or pressure and (ii) with or without cough; for ≥12 weeks.

In one embodiment, the subject in need of treatment has difficult-to-treat CRS, i.e., the subject has persistent symptoms of CRS despite being treated with recommended medication (e.g., intranasal corticosteroid treatment and up to two short courses of antibiotics or systemic corticosteroids in the past year) and surgery. Difficult-to-treat CRS is also referred to herein as “refractory CRS”.

In one embodiment, the subject in need of treatment has primary CRS, which may be localized primary CRS or diffuse primary CRS. In another embodiment, the subject in need of treatment has secondary CRS, which may be localized secondary CRS or diffuse secondary CRS (Fokkens et al. (2020). Rhinology. 58(2), 82-111, incorporated herein by reference in its entirety).

In some embodiments, the level of DPP1 in a biological sample obtained from the subject is in the range of about 1 ng/mL to about 1000 ng/mL prior to the administration period. The disclosure further provides methods of treating CRSin a subject in need thereof, comprising: (a) determining the level of DPP1 in a biological sample obtained from the subject, and (b) administering to the subject, a pharmaceutical composition comprising an effective amount of any one of the compounds disclosed herein. In some embodiments, the level of DPP1 determined in step (a), or the level of DPP1 in a biological sample obtained from the subject prior to the administration period is in the range of about 1 ng/mL to about 1000 ng/mL, for example about 3 ng/mL, about 5 ng/mL, about 7 ng/mL, about 10 ng/mL, about 13 ng/mL, about 15 ng/mL, about 17 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, or about 1000 ng/mL, including all values and subranges that lie therebetween. In some embodiments, the level of DPP1 determined in step (a), or the level of DPP1 in a biological sample obtained from the subject before administration of the composition is in the range of about 5 ng/mL to about 20 ng/mL. In some embodiments, the level of DPP1 determined in step (a), or the level of DPP1 in a biological sample obtained from the subject before administration of the composition is in the range of about 1 ng/mL to about 100 ng/mL.

In some embodiments, the level or activity of DPP1 determined in step (a), or the level or activity of DPP1 in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) higher than the level or activity, respectively, of DPP1 in a control subject who does not have CRS.

In some embodiments, the level or activity of DPP1 determined in step (a), or the level or activity of DPP1 in a biological sample obtained from the subject before administration of the composition is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900% or about 1000%, including all values and subranges that lie therebetween) higher than the level or activity, respectively, of DPP1 in a control subject who does not have CRS.

In some embodiments, the activity of DPP1 in a biological sample obtained from the subject during or subsequent to the administration period is less than: (a) the activity of DPP1 in the biological sample obtained from the subject before administration of the composition, and/or (b) the activity of DPP1 in a biological sample obtained from a control subject, wherein the control subject has CRS and is not administered the composition. In some embodiments, the activity of DPP1 in a biological sample obtained from the subject during the administration period or subsequent to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, including all values and subranges that lie therebetween) lower than: (a) the activity of DPP1 in the biological sample obtained from the subject prior to the administration period, and/or (b) the activity of DPP1 in a biological sample obtained from a control subject, wherein the control subject has CRS and is not administered the pharmaceutical composition.

In some embodiments, the level of DPP1 in a biological sample obtained from the subject during or subsequent to the administration period is less than: (a) the level of DPP1 in the biological sample obtained from the subject before administration of the composition, and/or (b) the level of DPP1 in a biological sample obtained from a control subject, wherein the control subject has CRS and is not administered the composition. In some embodiments, the level of DPP1 in a biological sample obtained from the subject during or subsequent to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, including all values and subranges that lie therebetween) lower than: (a) the level of DPP1 in the biological sample obtained from the subject prior to the administration period, and/or (b) the level of DPP1 in a biological sample obtained from a control subject, wherein the control subject has CRS and is not administered the pharmaceutical composition.

In some embodiments, DPP1 and its levels may be detected and/or quantified using methods, such as, for example, western blotting and enzymatic activity assays. Further details are provided in Pham et al. (1999). Proc Natl Acad Sci 96(15), 8627-32; Chen et al. (2021). J. Med. Chem. 64, 11857-11885; Hamon et al. (2016). J Biol Chem. 291(16), 8486-99; and International Patent Application Publication No. 2021/154812, the contents of each of which is incorporated herein by reference in their entireties.

In some embodiments, the level of neutrophil extracellular traps (NETs) in a biological sample obtained from the subject is in the range of about 1 ng/mL to about 1000 ng/mL prior to the administration period. The disclosure further provides methods of treating CRS in a subject in need thereof, comprising: (a) determining the level of NETs in a biological sample obtained from the subject, and (b) administering to the subject, a pharmaceutical composition comprising an effective amount of any one of the compounds disclosed herein for an administration period. In some embodiments, the level of NETs determined in step (a), or the level of neutrophil extracellular traps (NETs) in a biological sample obtained from the subject prior to the administration period is in the range of about 1 ng/mL to about 1000 ng/mL, for example about 3 ng/mL, about 5 ng/mL, about 7 ng/mL, about 10 ng/mL, about 13 ng/mL, about 15 ng/mL, about 17 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, or about 1000 ng/mL, including all values and subranges that lie therebetween.

In some embodiments, the level of NETs determined in step (a), or the level of NETs in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) higher than the level of NETs in a healthy subject who does not have CRS.

In some embodiments, the level of NETs determined in step (a), or the level of NETs in a biological sample obtained from the subject prior to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900% or about 1000% including all values and subranges that lie therebetween) higher than the level of NETs in a control subject who does not have CRS.

In some embodiments, the level of neutrophil extracellular traps (NETs) in a biological sample obtained from the subject during or subsequent to the administration period is less than: (a) the level of NETs in the biological sample obtained from the subject prior to the administration period, and/or (b) the level of NETs in a biological sample obtained from a control subject, wherein the control subject has CRS and is not administered the pharmaceutical composition. In some embodiments, the level of NETs in a biological sample obtained from the subject during the administration period or subsequent to the administration period is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, including all values and subranges that lie therebetween) lower than: (a) the level of NETs in the biological sample obtained from the subject prior to the administration period, and/or (b) the level of NETs in a biological sample obtained from a control subject, wherein the control subject has CRS and is not administered the pharmaceutical composition.

In some embodiments, the level of NETs is the level of circulating plasma NETs. In some embodiments, the level of NETs is determined by measuring DNA-complexed with NET-molecules like myeloperoxidase (MPO-DNA) or neutrophil elastase (NE-DNA) using enzyme-linked immunosorbent assays (ELISAs), measuring the presence of citrullinated histones by fluorescence microscopy, flow cytometric detection of NET-components, immunofluorescence to detect the colocalization of NET-associated molecules (NE, MPO, CitH3) with extracellular DNA or using flow cytometry or confocal microscopy-based methods. Further details on determining the levels of NETs is provided in Arends et al. (2019). J. Vis. Exp. 143, e59150; Gil et al. (2020). Sci Rep 10, 4320; and Thalin, et al. (2019). Arteriosclerosis, Thrombosis, and Vascular Biology 39, 1724-1738, each of which is incorporated herein by reference in its entirety for all purposes.

In some embodiments, the level of a neutrophil serine protease (NSP) in a biological sample obtained from the subject is in the range of about 1 ng/mL to about 1000 ng/mL prior to the administration period. The disclosure further provides methods of treating CRS in a subject in need thereof, comprising: (a) determining the level of a NSP in a biological sample obtained from the subject, and (b) administering to the subject, a pharmaceutical composition comprising an effective amount of any one of the compounds disclosed herein for an administration period. In some embodiments, the level of the NSP determined in step (a), or the level of the NSP in a biological sample obtained from the subject prior to the administration period is in the range of about 1 ng/mL to about 1000 ng/mL, for example about 3 ng/mL, about 5 ng/mL, about 7 ng/mL, about 10 ng/mL, about 13 ng/mL, about 15 ng/mL, about 17 ng/mL, about 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, about 650 ng/mL, about 700 ng/mL, about 750 ng/mL, about 800 ng/mL, about 850 ng/mL, about 900 ng/mL, about 950 ng/mL, or about 1000 ng/mL, including all values and subranges that lie therebetween.

In some embodiments, the level or activity of the NSP determined in step (a), or the level or activity of the NSP in a biological sample obtained from the subject prior to the administration period is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 25 fold, or about 30 fold, including all values and subranges that lie therebetween) higher than the level or activity, respectively, of the NSP in a control subject who does not have CRS.

In some embodiments, the level or activity of the NSP determined in step (a), or the level or activity of the NSP in a biological sample obtained from the subject prior to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 200%, at least about 300%, at least about 400%, at least about 500%, at least about 600%, at least about 700%, at least about 800%, at least about 900% or at least about 1000% including all values and subranges that lie therebetween) higher than the level or activity, respectively of the NSP in a control subject who does not have CRS.

In some embodiments, the activity of a neutrophil serine protease (NSP) in a biological sample obtained from the subject during or subsequent to the administration period is less than: (a) the activity of the NSP in the biological sample obtained from the subject prior to the administration period, and/or (b) the activity of the NSP in a biological sample obtained from a control subject, wherein the control subject has CRS and is not administered the pharmaceutical composition. In some embodiments, the activity of the NSP in a biological sample obtained from the subject during or subsequent to the administration period is at least about 2% (for example, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) lower than: (a) the activity of the NSP in the biological sample obtained from the subject prior to the administration period, and/or (b) the activity of the NSP in a biological sample obtained from a control subject, wherein the control subject has CRS and is not administered the pharmaceutical composition.

In some embodiments, the level of a neutrophil serine protease (NSP) in a biological sample obtained from the subject during or subsequent to the administration period is less than: (a) the level of the NSP in the biological sample obtained from the subject prior to the administration period, and/or (b) the level of the NSP in a biological sample obtained from a control subject, wherein the control subject has CRS and is not administered the pharmaceutical composition. In some embodiments, the level of the NSP in a biological sample obtained from the subject during or subsequent to the administration period is at least about 2% (for example, at least about 3%, about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, including all values and subranges that lie therebetween) lower than: (a) the level of the NSP in the biological sample obtained from the subject prior to the administration period, and/or (b) the level of the NSP in a biological sample obtained from a control subject, wherein the control subject has CRS and is not administered the pharmaceutical composition.

In some embodiments, the NSP is secreted NSP, or NSP in the cytoplasmic granules. In some embodiments, the NSP is the proform of the NSP. In some embodiments, the NSP is the active form of the NSP. In some embodiments, the NSP is the secreted proform of the NSP. In some embodiments, the NSP is neutrophil elastase (NE), proteinase 3 (PR3), cathepsin G (CatG), neutrophil serine protease 4 (NSP4), or any combination thereof. In some embodiments, the NSP is cell surface-localized NSP, intracellular NSP, or a combination thereof. In some embodiments, the level of the cell surface-localized NSP or intracellular NSP is measured using flow cytometry.

In some embodiments, neutrophil elastase (NE), proteinase 3 (PR3), cathepsin G (CatG), or levels thereof may be detected and/or quantified using methods, such as, for example, western blotting, ELISA assays, enzymatic activity assays, or any combination thereof. Non-limiting examples of ELISA assays include ProteaseTag® Active NE Immunoassay, ProteaseTag® Active PR3 Immunoassay, and ProteaseTag® Active CatG Immunoassay from ProAxsis (Belfast, Northern Ireland). Non-limiting examples of activity assays include NE enzymatic kinetic assays, PR3 enzymatic kinetic assays, and CatG enzymatic kinetic assays. In some embodiments, NSP4 and its levels may be detected and/or quantified using methods, such as, for example, western blotting and enzymatic activity assays. Further details are provided in Perera et al. (2012). PNAS 109, 6229-6234; Perera et al. (2013). J Immunol 191, 2700-2707; Kasperkiewicz et al. (2015). PLoS One 10(7):e0132818; each of which is incorporated herein by reference in its entirety.

In some embodiments, the biological sample comprises sinonasal tissue, blood, serum, white blood cells (WBCs), neutrophils, or any combination thereof.

In the methods of treating CRS provided herein, the pharmaceutical composition is administered by a suitable administration route. Administration routes include oral, enteral, transmucosal, rectal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular (including administration to skeletal, diaphragm and/or cardiac muscle), intradermal, intrapleural, intracerebral, intraarticular, intravascular or via infusion), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). In some embodiments, the administration is by injection into the central nervous system. In one embodiment, administration is via the enteral route and is conducted through a nasogastric (NG) tube.

In a preferred embodiment, the administration route is oral. In a further embodiment, the administration is once-daily oral administration. In even a further embodiment, the pharmaceutical composition comprises 10 mg, 25 mg or 40 mg of a compound of Formula (I). In yet even a further embodiment, the compound of Formula (I) is brensocatib.

The length of the administration period in any given case may depend on the nature and severity of the CRS being treating or prevented and may be determined by the prescribing physician. In some embodiments, the administration period is about 30 days, about 35 days, about 40 days, about 45 days, about 50 days, about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 30 months, about 36 months, about 4 years, about 5 years, about 10 years, about 15 years or about 20 years. In some embodiments, the compounds or compositions disclosed herein may be administered for a period of about 24 weeks. In some embodiments, the compounds or compositions disclosed herein may be administered for a period of about 52 weeks. In yet another embodiment, the administration period is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 30 months, at least about 36 months, at least about 4 years, at least about 5 years, at least about 10 years, at least about 15 years or at least about 20 years.

In some embodiments, the administration period for the methods provided herein is at least about 30 days, at least about 35 days, at least about 40 days, at least about 45 days, at least about 50 days, at least about 2 months, at least about 3 months, at least about 4 months or at least about 6 months, at least about 7 months, at least about 8 months, at least about 9 months, at least about 10 months, at least about 11 months, at least about 1 year, at least about 2 years, at least about 3 years, at least about 4 years, at least about 5 years. The administration period for the methods provided herein, in another embodiment, is from about 30 days to about 180 days. In another embodiment, the administration period is from about 30 days to about 36 months, or from about 30 days to about 30 months, or from about 30 days to about 24 months, or from about 30 days to about 18 months, or from about 30 days to about 12 months, or from about 30 days to about 6 months, or from about 6 months to about 30 months, or from about 6 months to about 24 months, or from about 6 months to about 18 months, or from about 12 months to about 36 months, or from about 12 months to about 24 months.

In one embodiment, the administration period is from about 1 year to about 50 years. For example, the administration period, in one embodiment, is from about 1 year to about 40 years, from about 1 year to about 25 years, 1 year to about 20 years, from about 1 year to about 15 years, from about 1 year to about 10 years, from about 1 year to about 5 years, from about 1 year to about 3 years, from about 1 year to about 2 years, from about 2 years to about 15 years, from about 2 year to about 10 years, from about 2 years to about 8 years, from about 2 years to about 5 years, from about 2 years to about 4 years, or from about 2 years to about 3 years.

In one embodiment, the subject is administered the pharmaceutical composition chronically. That is, the subject is administered the composition for their entire life, once treatment for CRS is initiated.

In some embodiments, the pharmaceutical composition may be administered to the subject once a day or more than once a day during the administration period. In some embodiments, the composition may be administered to the subject twice a day during the administration period. In some embodiments, the composition may be administered to the subject every day, every other day, every third day, every fourth day, every fifth day, or every sixth day during the administration period. In some embodiments, the pharmaceutical composition may be administered to the subject weekly, bi-weekly or every three weeks during the administration period. In some embodiments, the pharmaceutical composition is administered at approximately the same time every day during the administration period.

In a preferred embodiment, administration of the pharmaceutical composition is once daily. In another embodiment, administration of the pharmaceutical composition is twice daily. In another embodiment, administration is once weekly, twice weekly, thrice weekly, four times weekly, five times weekly or six times weekly.

The compounds of Formula (I), or pharmaceutically acceptable salts thereof, may be used on their own, or in conjunction with the standard-of-care administered by a treating physician. Any standard-of-care therapeutic may be used in combination with the compounds disclosed herein. In some embodiments, the subject has undergone or will undergo surgery for treating the CRS.

In some embodiments, the compounds of Formula (I), or pharmaceutically acceptable salts thereof, are administered in combination with a secondary therapy to the subject. In some embodiments, the secondary therapy comprises one or more of the following: a steroid, an antihistamine, an antibiotic, an anti-depressant, a biologic, an anti-leukotriene, nasal saline irrigation, and a surgical intervention. In some embodiments, the steroid is fluticasone propionate or mometasone furoate. In a further embodiment, the steroid is mometasone furoate. In some embodiments, the steroid is administered topically, intranasally, systemically, or orally. Non-limiting examples of antihistamines include pseudoephedrine, loratadine, cetirizine, and diphenhydramine. In some embodiments, the antibiotic is a macrolide antibiotic. In some embodiments, the macrolide antibiotic is erythromycin, roxithromycin, azithromycin or clarithromycin. In some embodiments, the anti-depressant is desipramine, or fluoxetine. In some embodiments, the biologic is dupilumab, omalizumab, benralizumab, reslizumab, or mepolizumab. Non-limiting examples of anti-leukotrienes include montelukast, zafirlukast, and pranlukast, and 5-lipoxygenase inhibitors (such as, zileuton). In some embodiments, the surgical intervention is functional endoscopic sinus surgery (FESS).

The term administered “in combination,” as used herein, is understood to mean that two (or more) different treatments are delivered to the subject during the course of the subject's affliction with the disorder (such as CRS), such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins, which may be referred to as “sequential” or “serial” delivery.

In some embodiments, the treatment is more effective because of combined administration. For example, the second treatment is more effective; for e.g., an equivalent effect is seen with less of the second treatment, or the second treatment reduces symptoms to a greater extent, than would be seen if the second treatment were administered in the absence of the first treatment, or the analogous situation is seen with the first treatment. The effect of the two treatments can be partially additive, wholly additive, or greater than additive (synergistic).

The compositions employed herein include an effective amount of any one or more of the compounds disclosed herein, or pharmaceutically acceptable salts thereof.

The compounds of Formula (I), or pharmaceutically acceptable salts thereof, may be used on their own, but will generally be administered in the form of a pharmaceutical composition in which the Formula (I) compound/salt (active pharmaceutical ingredient (API)) is in a pharmaceutical composition comprising a pharmaceutically acceptable adjuvant(s), diluents(s) and/or carrier(s). In one embodiment, the pharmaceutical composition is one of the pharmaceutical compositions described in International Application Publication No. WO 2019/166626, the disclosure of which is incorporated herein by reference in its entirety for all purposes.

Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002, incorporated by reference herein in its entirety for all purposes. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

Depending on the mode of administration, the pharmaceutical composition may comprise from about 0.05 to about 99 wt %, for example, from about 0.05 to about 80 wt %, or from about 0.10 to about 70 wt %, or from about 0.10 to about 50 wt %, of API, all percentages by weight being based on the total weight of the pharmaceutical composition. Unless otherwise provided herein, API weight percentages provided herein are for the respective free base form of the compound of Formula (I).

In one embodiment, the pharmaceutical composition is in the oral dosage form of a film-coated oral tablet. In another embodiment, the oral dosage form is an immediate release dosage form with rapid dissolution characteristics under in vitro test conditions. In one embodiment, the oral dosage form is administered once daily to reach the daily dosage disclosed herein. In a further embodiment, the oral dosage form is administered at approximately the same time every day, e.g., prior to breakfast. In another embodiment, the oral dosage form is administered 2x daily to reach the daily dosage disclosed herein.

In some embodiments, the compositions of the present disclosure are formulated using a pharmaceutically acceptable salt of a compound of Formula (I). Pharmaceutically-acceptable salts include, for example, acid addition salts derived from inorganic acids, e.g., hydrochloric or phosphoric acids, or from organic acids, e.g., acetic, oxalic, tartaric, mandelic, and the like. In some embodiments, the salts may be derived from inorganic bases (e.g., sodium, potassium, ammonium, calcium, or ferric hydroxides) or from organic bases (e.g., isopropylamine, trimethylamine, histidine, procaine) and the like.

The disclosure provides compositions comprising an effective amount of a compound of Formula (I), or a pharmaceutically acceptable salt thereof, for use in the methods provided herein. Any of the compounds provided herein may be used in a composition, for delivery via one of the methods provided herein.

In some embodiments, the composition is in a solid form, such as a lyophilized powder suitable for reconstitution, a liquid solution, suspension, emulsion, tablet, pill, capsule, sustained release formulation, or powder. In some embodiments, delivery vehicles such as liposomes, nanocapsules, microparticles, microspheres, lipid particles, vesicles, and the like, may be used.

In some embodiments, the compositions disclosed herein further comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

In some embodiments, the amount of a compound of Formula (I) present in a pharmaceutical composition depends on the mode of administration. In some embodiments, the pharmaceutical composition comprises from about 0.05% w to about 99% w (percent by weight), for example, about 0.5% w, about 1% w, about 5% w, about 10% w, about 15% w, about 20% w, about 25% w, about 30% w, about 35% w, about 40% w, about 45% w, about 50% w, about 55% w, about 60% w, about 65% w, about 70% w, about 75% w, about 80% w, about 85% w, about 90% w, about 95% w, about 98% w, or about 99% w, including all values and subranges that lie therebetween. In some embodiments, the pharmaceutical composition comprises from about 0.05% w to about 80% w, or from about 0.10% w to about 70% w, or from about 0.10% w to about 50% w, of active ingredient (compound of Formula (I)), all percentages by weight being based on total composition.

In some embodiments, the adjuvant(s), diluent(s) or carrier(s) present in the pharmaceutical composition are selected based on the mode of administration. For instance, for oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the above-mentioned excipients for tablets. Also, liquid or semisolid formulations of the compound of the disclosure may be filled into hard gelatine capsules.

In some embodiments, the form of the pharmaceutical composition depends on the mode of administration. For instance, in one oral administration embodiment, the oral dosage form is a film-coated oral tablet. In a further embodiment, the dosage form is an immediate release dosage form with rapid dissolution characteristics under in vitro test conditions.

In one embodiment, the composition is an oral disintegrating tablet (ODT). ODTs differ from traditional tablets in that they are designed to be dissolved on the tongue rather than swallowed whole.

In one embodiment, the composition is an oral thin film or an oral disintegrating film (ODF). Such formulations, when placed on the tongue, hydrate via interaction with saliva, and release the active compound from the dosage form. The ODF, in one embodiment, contains a film-forming polymer such as hydroxypropylmethylcellulose (HPMC), hydroxypropyl cellulose (HPC), pullulan, carboxymethyl cellulose (CMC), pectin, starch, polyvinyl acetate (PVA) or sodium alginate.

Liquid preparations for oral administration may be in the form of syrups, solutions or suspensions. Solutions, for example, may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

The compounds of the disclosure may be prepared, in known manner, in a variety of ways. For example, in one embodiment, compounds of Formula (I) are prepared according to the methods set forth in U.S. Pat. No. 9,522,894, incorporated by reference herein in its entirety for all purposes.

As provided throughout, according to the methods provided herein, a compound of Formula (I) can be administered as a pharmaceutically acceptable salt. A pharmaceutically acceptable salt of a compound of Formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound of Formula (I).

Where the compound of Formula (I) is sufficiently acidic, pharmaceutically acceptable salts include, but are not limited to, an alkali metal salt, e.g., Na or K, an alkali earth metal salt, e.g., Ca or Mg, or an organic amine salt. Where the compound of Formula (I) is sufficiently basic, pharmaceutically acceptable salts include, but are not limited to, inorganic or organic acid addition salts.

There may be more than one cation or anion depending on the number of charged functions and the valency of the cations or anions. For reviews on suitable salts, and pharmaceutically acceptable salts amenable for use herein, see Berge et al. (1977). J. Pharm. Sci., 66, 1-19 or “Handbook of Pharmaceutical Salts: Properties, selection and use”, P. H. Stahl, P. G. Vermuth, IUPAC, Wiley-VCH, 2002, incorporated by reference herein in its entirety for all purposes. The compounds of Formula (I) may form mixtures of its salt and co-crystal forms. It is also to be understood that the methods provided herein can employ such salt/co-crystal mixtures of the compound of Formula (I).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

In another embodiment of the methods, the pharmaceutical composition administered to the patient is Composition (A) comprising:
(a) from about 1 to about 30 wt % of the compound of Formula (I), or a pharmaceutically acceptable salt thereof;
(b) from about 45 to about 85 wt % of a pharmaceutical diluent;
(c) from about 6 to about 30 wt % of a compression aid;
(d) from about 1 to about 15 wt % of a pharmaceutical disintegrant;
(e) from about 0.00 to about 2 wt % of a pharmaceutical glidant; and
(f) from about 1 to about 10 wt % of a pharmaceutical lubricant;
wherein the components add up to 100 wt %.

In a further embodiment, the compound of Formula (I) is brensocatib. In one embodiment, brensocatib is in polymorphic Form A as disclosed in U.S. Pat. No. 9,522,894. In another embodiment, brensocatib is characterized by one of the X-ray powder diffraction patterns described in International Application Publication No. WO 2019/166626.

In some embodiments of the methods, Composition (A) comprises the compound of Formula (I), e.g., brensocatib, in an amount from about 1 to about 25 wt %; from about 1 to about 20 wt %; from about 1 to about 15 wt %; from about 1 to about 10 wt %; from about 1 to about 5 wt %, or from about 1 to about 3 wt % of the total weight of the composition.

In some embodiments of the methods, Composition (A) comprises the compound of Formula (I), e.g., brensocatib, in an amount from about 1.5 to about 30 wt %; from about 1.5 to about 25 wt %; from about 1.5 to about 20 wt %; from about 1.5 to about 15 wt %; from about 1.5 to about 10 wt %; or from about 1.5 to about 5 wt % of the total weight of the composition.

In some embodiments of the methods, Composition (A) comprises the compound of Formula (I), e.g., brensocatib, in an amount from about 3 to about 30 wt %; from about 3 to about 25 wt %; from about 3 to about 20 wt %; from about 3 to about 15 wt %; from about 3 to about 10 wt %; or from about 3 to about 5 wt % of the total weight of the composition. In a further embodiment, the compound of Formula (I) is present at from about 3 to about 10 wt % of the total weight of the composition. In a further embodiment, the compound of Formula (I) is brensocatib, or a pharmaceutically acceptable salt thereof.

In some embodiments of the methods, Composition (A) comprises the compound of Formula (I), e.g., brensocatib, in an amount of about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt % or about 30 wt % of the total weight of the composition.

In some embodiments of the methods, Composition (A) comprises one or more pharmaceutical diluents selected from the group consisting of microcrystalline cellulose, calcium carbonate, calcium phosphate, calcium sulfate, cellulose acetate, erythritol, ethylcellulose, fructose, inulin, isomalt, lactitol, lactose, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose, xylitol, and a combination of the foregoing. In one embodiment, Composition (A) comprises two or more pharmaceutical diluents. In another embodiment, Composition (A) comprises one pharmaceutical diluent. In a further embodiment, the pharmaceutical diluent is microcrystalline cellulose. Microcrystalline cellulose is a binder/diluent in oral tablet and capsule formulations and can be used in dry-granulation, wet-granulation, and direct-compression processes.

In some embodiments of the methods, Composition (A) comprises one or more pharmaceutical diluents in an amount from about 45 to about 80 wt %, from about 45 to about 75 wt %, from about 45 to about 70 wt %, from about 45 to about 65 wt %, from about 45 to about 60 wt %, or from about 45 to about 55 wt % of the total weight of the composition. In a further embodiment, the one or more pharmaceutical diluents comprises microcrystalline cellulose. In even a further embodiment, the compound of Formula (I) is brensocatib, or a pharmaceutically acceptable salt thereof.

In some embodiments of the methods, Composition (A) comprises one or more pharmaceutical diluents in an amount from about 50 to about 85 wt %, from about 50 to about 75 wt %, from about 55 to about 85 wt %, from about 55 to about 70 wt %, from about 60 to about 85 wt %, from about 65 to about 85 wt %, from about 70 to about 85 wt %, or from about 75 to about 85 wt % of the total weight of the composition. In a further embodiment, the one or more pharmaceutical diluents is present at from about 55 to about 70 wt % of the total weight of the composition. In a further embodiment, the one or more pharmaceutical diluents comprises microcrystalline cellulose. In even a further embodiment, the compound of Formula (I) is brensocatib, or a pharmaceutically acceptable salt thereof.

In some embodiments of the methods, Composition (A) comprises one or more pharmaceutical diluents in an amount of about 45 wt %, about 50 wt %, about 55 wt %, about 60 wt %, about 65 wt %, about 70 wt %, about 75 wt %, about 80 wt % or about 85 wt % of the total weight of the composition.

In some embodiments of the methods, the one or more pharmaceutical diluents in Composition (A) is microcrystalline cellulose. In other embodiments, the one or more pharmaceutical diluents comprises calcium carbonate, calcium phosphate, calcium sulfate, cellulose acetate, erythritol, ethylcellulose, fructose, inulin, isomalt, lactitol, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose and xylitol.

In the present disclosure, the terms “disintegrant” and “disintegrants” are intended to be interpreted in the context of pharmaceutical formulation science. Accordingly, a disintegrant in the Composition (A) may be, for example: alginic acid, calcium alginate, carboxymethylcellulose calcium, chitosan, croscarmellose sodium, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, sodium starch glycolate, starch, or a combination thereof.

In some embodiments of the methods, the one or more disintegrants in Composition (A) is sodium starch glycolate. In one embodiment, the amount of the disintegrants present in Composition (A) is between 2% and 8% of the total weight of the composition. In a further embodiment, the amount of the disintegrants is about 2 wt %, about 2.5 wt %, about 3 wt %, about 3.5 wt %, about 4 wt % or about 4.5 wt % of the total weight of the composition. The physical properties of sodium starch glycolate, and hence its effectiveness as a disintegrant, are affected by the degree of crosslinkage, extent of carboxymethylation, and purity.

In some embodiments of the methods, the one or more pharmaceutical disintegrants in Composition (A) comprises croscarmellose sodium.

In some embodiments of the methods, Composition (A) comprises one or more pharmaceutical disintegrants in an amount from about 2 to about 14 wt %, from about 2 to about 13 wt %, from about 2 to about 12 wt %, from about 2 to about 11 wt %, from about 2 to about 10 wt %, from about 2 to about 9 wt %, from about 2 to about 8 wt %, from about 2 to about 7 wt %, from about 2 to about 6 wt %, from about 2 to about 5 wt %, from about 3.5 to about 4.5 wt % of the total weight of the composition. In a further embodiment, the one or more pharmaceutical disintegrants is present at from about 3.5 to about 4.5 wt % of the total weight of the pharmaceutical composition. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) is brensocatib, or a pharmaceutically acceptable salt thereof.

In the present disclosure, the terms “glidants” and “gliding agents” are intended to be interpreted in the context of pharmaceutical formulation science. Accordingly, a glidant in Composition (A) may be, for example: silicon dioxide, colloidal silicon dioxide, powdered cellulose, hydrophobic colloidal silica, magnesium oxide, magnesium silicate, magnesium trisilicate, sodium stearate and talc.

Accordingly, in some embodiments of the methods, the one or more pharmaceutical glidants in Composition (A) is selected from silicon dioxide, colloidal silicon dioxide, powdered cellulose, hydrophobic colloidal silica, magnesium oxide, magnesium silicate, magnesium trisilicate, sodium stearate, talc, or a combination of the foregoing. In one embodiment, the glidant is silicon dioxide. Its small particle size and large specific surface area give it desirable flow characteristics that are exploited to improve the flow properties of dry powders in a number of processes such as tableting and capsule filling. Typical silicon dioxide concentrations for use herein range from about 0.05 to about 1.0 wt %. Porous silica gel particles may also be used as a glidant, which may be an advantage for some formulations, with typical concentrations of 0.25-1%.

In some embodiments of the methods, Composition (A) comprises one or more pharmaceutical glidants in an amount from about 0.00 to about 1.75 wt %; from about 0.00 to about 1.50 wt %; from about 0.00 to about 1.25 wt %; from about 0.00 to about 1.00 wt %; from about 0.00 to about 0.75 wt %; from about 0.00 to about 0.50 wt %; from about 0.00 to about 0.25 wt %; from about 0.00 to about 0.20 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.05 to about 1.75 wt %; from about 0.05 to about 1.50 wt %; from about 0.05 to about 1.25 wt %; from about 0.05 to about 1.00 wt %; from about 0.05 to about 0.75 wt %; from about 0.05 to about 0.50 wt %; from about 0.05 to about 0.25 wt %; or from about 0.05 to about 0.20 wt % of the total weight of the composition. In a further embodiment, the one or more pharmaceutical glidants is present at from about 0.05 to about 0.25 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound Formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.10 to about 2 wt %; from about 0.2 to about 2 wt %; from about 0.3 to about 2 wt %; or from about 0.40 to about 2 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In the present disclosure, the terms “lubricant” and “lubricants”, as used herein, are intended to be interpreted in the context of pharmaceutical formulation science. Accordingly, a lubricant may be, for example calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate.

Accordingly, in some embodiments of the methods, the one or more pharmaceutical lubricants in Composition (A) are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate. In other embodiments, the one or more pharmaceutical lubricants are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, stearic acid, talc, tribehenin and zinc stearate.

In some embodiments of the methods, Composition (A) comprises one or more pharmaceutical lubricants and the lubricant is not sodium stearyl fumarate. In a further embodiment, the compound of Formula (I) in Composition (A) is brensocatib, or a pharmaceutically acceptable salt thereof.

In one embodiment of the methods, Composition (A) includes glycerol behenate as the lubricant.

In some embodiments of the methods, the one or more pharmaceutical lubricants in Composition (A) comprises glyceryl behenate, magnesium stearate, stearic acid, or a combination thereof.

In one embodiment of the methods, the lubricant in Composition (A) is glyceryl behenate, magnesium stearate, or a combination thereof.

In one embodiment of the methods, the one or more pharmaceutical lubricants in Composition (A) comprises sodium stearyl fumarate and/or one or more behenate esters of glycerine.

In some embodiments of the methods, Composition (A) comprises one or more pharmaceutical lubricants in an amount from about 1 wt % to about 9 wt %, from about 1 wt % to about 8 wt %, from about 1 wt % to about 7 wt %, from about 1 wt % to about 6 wt %, from about 1 wt % to about 5 wt %, from about 2 wt % to about 10 wt %, from about 2.5 wt % to about 10 wt %, from about 2 wt % to about 8 wt %, from about 2 wt % to about 7 wt %, from about 2 wt % to about 6 wt %, from about 2 wt % to about 5 wt %, from about 2 wt % to about 4.5 wt %, or from about 2.5 wt % to about 4.5 wt % of the total weight of the composition. In a further embodiment, the one or more pharmaceutical lubricants is present at from about 2.5 to about 4.5 wt % of the total weight of the composition. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of Formula (I) in Composition (A) is brensocatib, or a pharmaceutically acceptable salt thereof.

In one embodiment of the methods, the one or more pharmaceutical lubricants in Composition (A) consists of sodium stearyl fumarate and/or one or more behenate esters of glycerine or a mixture thereof.

In another embodiment of the methods, the one or more pharmaceutical lubricants in Composition (A) consists of sodium stearyl fumarate, glyceryl dibehenate, glyceryl behenate, tribehenin or any mixture thereof.

In one embodiment of the methods, the one or more pharmaceutical lubricants in Composition (A) comprises sodium stearyl fumarate. In another embodiment, the one or more pharmaceutical lubricants in Composition (A) consists of sodium stearyl fumarate.

In one embodiment of the methods, the one or more pharmaceutical lubricants in Composition (A) comprises one or more behenate esters of glycerine (i.e., one or more of glyceryl dibehenate, tribehenin and glyceryl behenate).

In one embodiment of the methods, the compression aid in Composition (A) is dicalcium phosphate dihydrate (also known as dibasic calcium phosphate dihydrate) (DCPD). DCPD is used in tablet formulations both as an excipient and as a source of calcium and phosphorus in nutritional supplements.

In one embodiment of the methods, Composition (A) comprises the compression aid, e.g., DCPD, in an amount from about 10 to about 30 wt %, including about 16 wt %, about 17 wt %, about 18 wt %, about 19 wt %, about 20 wt %, about 21 wt %, about 22 wt %, about 23 wt %, or about 24 wt % of the total weight of the composition. In a further embodiment, the compression aid is present at about 20 wt % of the total weight of the composition.

In one embodiment of the methods, Composition (A) comprises the compression aid, e.g., DCPD, in an amount from about 10 to about 25 wt %, from about 10 to about 20 wt %, from about 10 to about 15 wt %, from about 15 to about 25 wt %, or from about 20 to about 25 wt %, or from about 18 to about 22 wt % of the total weight of the composition. In a further embodiment, the compression aid is present at from about 18 to about 22 wt % of the total weight of the composition. In a further embodiment, the compression aid is DCPD. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of Formula (I) in the exemplary composition is brensocatib, or a pharmaceutically acceptable salt thereof.

In one embodiment of the methods, the pharmaceutical composition administered to the patient is Composition (B) comprising:
(a) from about 1 to about 30 wt % of the compound of Formula (I), or a pharmaceutically acceptable salt thereof;
(b) from about 55 to about 75 wt % of a pharmaceutical diluent;
(c) from about 15 to about 25 wt % of a compression aid;
(d) from about 3 to about 5 wt % of a pharmaceutical disintegrant;
(e) from about 0.00 to about 1 wt % of a pharmaceutical glidant; and
(f) from about 2 to about 6 wt % of a pharmaceutical lubricant;
wherein the components add up to 100 wt %.

In some embodiments of the methods where Composition (B) is administered to the patient, the identity of the pharmaceutical diluent, compression aid, pharmaceutical disintegrant, pharmaceutical glidant, and pharmaceutical lubricant in the composition may be one of those described above for Composition (A). In other embodiments, the amount of the pharmaceutical diluent, compression aid, pharmaceutical disintegrant, pharmaceutical glidant, and pharmaceutical lubricant in Composition (B) may also be one of those described above for Composition (A), as long as the amount is within the corresponding broader range recited above for Composition (B).

The pharmaceutical compositions disclosed herein, including Compositions (A) and (B), may be in a solid dosage form suitable for oral administration to a human being. For example, the pharmaceutical composition is a pharmaceutical tablet. Pharmaceutical tablets may be prepared using methods known to those skilled in the art including, for example, dry mixing/direct compression process as described in International Application Publication No. WO 2019/166626. In some embodiments, the pharmaceutical tablet comprises a tablet core wherein the tablet core comprises the pharmaceutical composition as disclosed herein and wherein the tablet core has a coating. In some embodiments, the coating is a film coating. The film coating may be applied using conventional methods known to those skilled in the art. A functional coating can be used to provide protection against, for example, moisture ingress or degradation by light. Additionally, a functional coating may be used to modify or control the release of the compound of Formula (I), e.g., brensocatib, from the composition. The coating may comprise, for example, about 0.2 to about 10 wt % of the total weight of the pharmaceutical composition, e.g., from about 0.2 to about 4 wt %, from about 0.2 to about 3 wt %, from about 1 to about 6 wt %, or from about 2 to about 5 wt % of the total weight of the pharmaceutical composition.

### Example

The present disclosure is further illustrated by reference to the following Example.

However, it should be noted that the Example, like the embodiments described above, is illustrative and is not to be construed as restricting the scope of the invention in any way.

### Example 1—A Phase 2b, Randomized, Double-Blind, Placebo-Controlled, Multicenter Study of the Efficacy and Safety of Brensocatib in Participants with Moderate to Severe Chronic Rhinosinusitis without Nasal Polyps (CRSsNP)

Brensocatib, a compound of Formula (I), is an oral reversible DPP1 inhibitor. DPP1 catalyzes activation of neutrophil serine proteases (NSPs) in neutrophils during neutrophil maturation in the bone marrow. NSPs are key agents of neutrophil-mediated inflammation, tissue damage, and excessive mucus production. By inhibiting DPP1, brensocatib prevents activation of NSPs and allows for neutrophils to mature and be released without active NSPs.

Therefore, brensocatib can potentially inhibit the neutrophilic inflammation component associated with CRSsNP by blocking DPP1.

This example describes a Phase 2b, randomized, double-blind, placebo-controlled, study to assess the efficacy and safety of brensocatib in participants with moderate to severe chronic rhinosinusitis without nasal polyps (CRSsNP). Brensocatib is the International Nonproprietary Name for (2S)-N-{(1S)-1-cyano-2-[4-(3-methyl-2-oxo-2,3-dihydro-1,3-benzoxazol-5-yl)phenyl]ethyl}-1,4-oxazepane-2-carboxamide

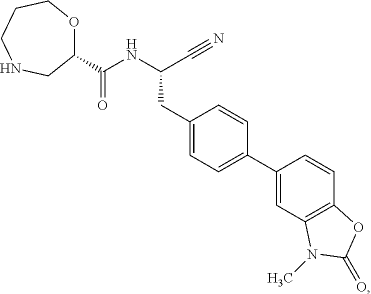 [SMILES: Cn1c(=O)oc2ccc(-c3ccc(C[C@@H](C#N)NC(=O)[C@@H]4CNCCCO4)cc3)cc21]

and is administered once daily (QD) for 24 weeks followed by a 4-week follow-up period in approximately 360 adult male and female participants (≥18 to ≤85 years of age) with moderate to severe CRSsNP. Eligible participants are randomized into three treatment arms in a 1:1:1 ratio, with approximately 120 participants per treatment arm, to receive once daily oral dosing of 10 mg brensocatib, 40 mg brensocatib, and matching placebo, respectively, for 24 weeks. Approximately 180 participants will have a blood eosinophil count of <300 cells/μL (the primary population) resulting in approximately 60 participants per treatment arm in the primary population, and approximately 180 participants will have a blood eosinophil count ≥300 but ≤750 cells/μL. Participants may be screened for study enrollment regardless of current intranasal mometasone furoate use or absence of history of intranasal mometasone furoate use. At the Screening Visit, all participants, regardless of history of use, will start treatment with intranasal mometasone furoate 200 μg twice a day (BID) or once daily (QD), if they cannot tolerate BID, throughout the study including the treatment period.

Brensocatib oral tablets are used in the study. The tablets are round, biconvex, and brown film-coated and are considered as an immediate-release dosage form. The tablets contain the equivalent of 10 mg or 40 mg of brensocatib drug substance and are identical in size and appearance. Each film-coated tablet contains active ingredient of brensocatib drug substance and compendial ingredients: microcrystalline cellulose, dibasic calcium phosphate dihydrate, sodium starch glycolate, silicon dioxide, and glyceryl behenate. The matching placebo without the active ingredient is a film-coated tablet identical to brensocatib film-coated tablets in shape, size, and color.

Efficacy parameters include Sinus Total Symptom Score (sTSS) for assessing nasal symptoms, Lund-MacKay (LMK) computed tomography (CT) scan score and CT Scan Volumetry for assessing CT sinus opacification, Sino Nasal Outcome Test (SNOT-22) for assessing quality of life, Nasal Congestion Scores (NCS) for assessing nasal congestion, (eDiary) Visual Analog Scale (VAS) scores for assessing nasal symptoms, Peak Nasal Inspiratory Flow (PNIF) for assessing nasal inspiratory flow, and the use of rescue therapy with systemic corticosteroids, antibiotics, or nasal surgery for assessing clinical worsening/acute rhinosinusitis. Without wishing to be bound by theory, administration of brensocatib may result in beneficial effects for patients suffering from CRSsNP via decreasing inflammation and mucus hypersecretion, leading to an improvement in symptoms (e.g., nasal congestion, facial pain, nasal discharge) and quality of life. Since brensocatib inhibits maturation of NSPs, without wishing to be bound by theory, brensocatib may provide a meaningful decrease of the inflammatory cascade.

### Subject Eligibility Criteria

TABLE 1
Inclusion criteria for the study

1. Male or female ≥18 years old and ≤85 years old.
2. At least a 12-week history (before Screening Visit) of
CRSsNP (confirmed by endoscopy) at Screening
3. Ongoing symptoms of nasal congestion, with an NCS
of ≥2 at Screening and for the weekly average score
in the week prior to randomization, and with sTSS (nasal
congestion, anterior/posterior rhinorrhea, facial
pain/pressure) ≥5 at Screening and for the weekly
average score in the week prior to randomization.
4. Blood eosinophil count of ≤750 cells/μL at Screening.
Participants with blood eosinophil count of ≤300 cells/μL
should have total IgE <150 IU/ml at Screening.
5. Previous sinonasal surgery for CRS and/or treatment
with systemic steroids or antibiotics for CRS within
a year of Screening Visit.
6. SNOT-22 score of ≥20 at Screening and Baseline Visits.
7. VAS score of ≥5 at Screening and Baseline Visits

### Study Design

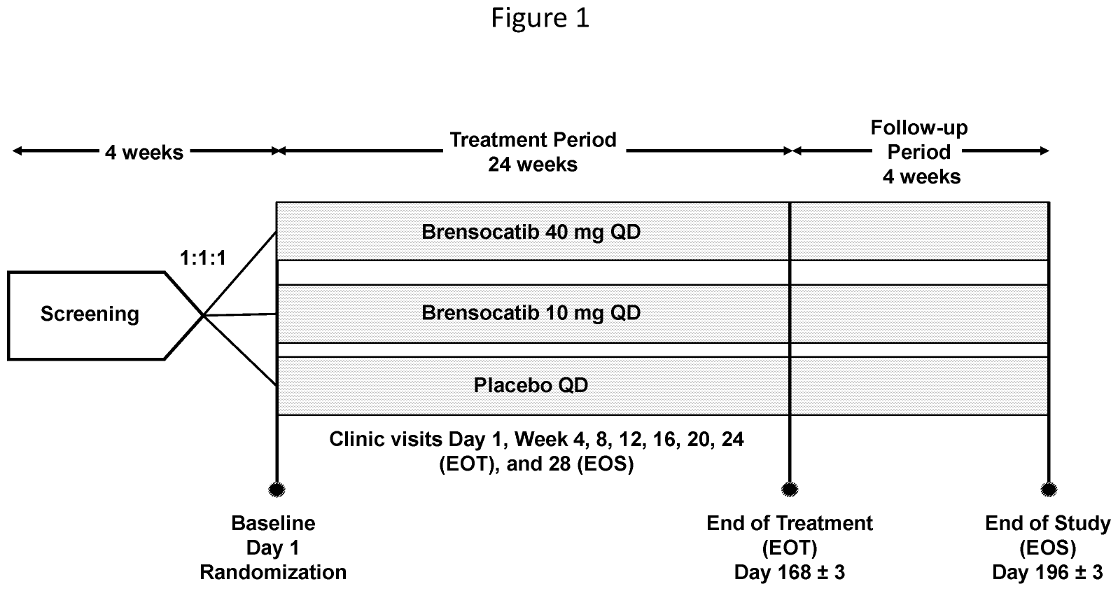

FIG. 1 provides a schematic diagram of the study design and treatment duration. In this randomized, double-blind, parallel-group, placebo-controlled study, brensocatib is administered once-daily for 24 weeks followed by a 4-week Follow-up Period in approximately 360 adult male and female participants (18 to 85 years of age) with moderate to severe CRSsNP. Investigators (including clinicians providing care to the participant), Sponsor, and participants/care providers are blinded to study treatment. Approximately 180 participants will have a blood eosinophil count of <300 cells/μL and approximately 180 participants will have a blood eosinophil count 300 but 750 cells/μL.

Randomization will be stratified by history of surgery for CRSsNP prior to screening (Yes/No), geographical area (North America, Europe, and Rest of World), and current diagnosis of asthma as comorbidity. An additional stratification factor for the population with eosinophil count 300 cells/μL will then be based on their cell counts (300-500 cells/μL vs 501-750 cells/μL). Stratification for eosinophil count will be based on the blood eosinophil count obtained at Screening.

The study includes 3 study periods:

Screening Period—Participant eligibility will be determined during a Screening Period of a minimum of 2 weeks and up to 4 weeks.

Treatment Period—Eligible participants will be randomized to receive double-blind brensocatib 10 mg, brensocatib 40 mg, or placebo film coated tablets once-daily by mouth for 24 weeks.

Follow-up Period—Participants will be followed for 4 weeks after the last dose of study treatment.

Participants may be Screened for study enrollment regardless of current intranasal mometasone furoate use or absence of history of intranasal mometasone furoate use. At the Screening Visit, all participants, regardless of history of use, will start treatment with intranasal mometasone furoate 200 μg BID or QD, if they cannot tolerate BID, throughout the study including the treatment period as background therapy.

A PD substudy will be conducted in a subset of participants. Nasal secretion samples will be collected from approximately 60 participants including: 30 participants from the population with blood eosinophil count <300 cells/μL and 30 participants from the population with blood eosinophil count ≥300 cells/μL at Screening. Samples will be collected at in-clinic visits at Day 1, Week 4, and Week 24.

The maximum study duration will be approximately 32 weeks for an individual participant, including a Screening Period of up to 4 weeks, a Double-Blind Treatment Period of 24 weeks, and a 4-week Follow-up Period.

### Outcomes Assessment

The objectives and endpoints of the study are shown in Table 2. Unless otherwise noted, the population attribute for the primary efficacy estimand is participants with CRSsNP who have baseline eosinophil counts <300 cells/μL. The entire population of participants with CRSsNP will be secondary for the efficacy estimands and primary for the safety, pharmacokinetic, and pharmacodynamic estimands. For all estimands, the treatment regimen is the randomized study treatment (i.e., brensocatib 10 mg QD, brensocatib 40 mg QD, or placebo QD). The PK objective is to evaluate systemic exposure of brensocatib in participants with CRSsNP.

TABLE 2
Objectives and Endpoints of the Study

| | | |
|---|---|---|
| | Variable | Population |
| Objectives | (or Endpoint) | Level Summary |

Primary

| | | |
|---|---|---|
| To evaluate the efficacy of | Change from Baseline to the 28- | Mean difference |
| brensocatib at 10 and 40 mg once | day average of daily Sinus Total | |
| daily (QD) compared with | Symptom Score (sTSS) at Week | |
| placebo in improving nasal | 24 | |
| symptoms | | |

Secondary

| | | |
|---|---|---|
| To evaluate the efficacy of | Change from Baseline in Lund- | Mean difference |
| brensocatib at 10 and 40 mg QD | MacKay (LMK) CT score at | |
| compared with placebo in | Week 24 | |
| improving computed tomography | Change from Baseline in | Mean difference |
| (CT) sinus opacification | percentage sinus opacification as | |
| | measured by volumetry at Week | |
| | 24 | |
| To evaluate the efficacy of | Change from Baseline in Sino- | Mean difference |
| brensocatib at 10 and 40 mg QD | Nasal Outcome Test (SNOT-22) | |
| compared with placebo in | at Week 24 | |
| improving quality of life | | |
| To evaluate the efficacy of | Change from Baseline to the 28- | Mean difference |
| brensocatib compared with | day average of daily Nasal | |
| placebo in improving nasal | Congestion Score (NCS) at | |
| congestion | Week 24 | |
| To evaluate the efficacy of | Change from Baseline to the 28- | Mean difference |
| brensocatib compared with | day average of daily (eDiary) | |
| placebo in improving nasal | Visual Analog Scale (VAS) | |
| symptoms | scores at Week 24 | |
| To evaluate the efficacy of | Change from Baseline to the 28- | Mean difference |
| brensocatib compared with | day average of daily Peak Nasal | |
| placebo in improving nasal | Inspiratory Flow (PNIF) at | |
| inspiratory flow | Week 24 | |
| To evaluate the efficacy of | Proportion of participants | Odds ratio |
| brensocatib in comparison to | requiring rescue (antibiotics, | |
| placebo in reducing clinical | systemic corticosteroids, and/or | |
| worsening/acute rhinosinusitis | nasal surgery)^{a} due to worsening | |
| | of any CRS symptom | |
| | Time-to first use of rescue | Hazard ratio |
| | (systemic corticosteroids, | |
| | antibiotics, and/or nasal surgery) | |
| | during the 24-week treatment | |
| | period | |
| To evaluate PK of brensocatib in | Brensocatib plasma | Mean |
| the CRSsNP population | concentration | concentrations |
| | | over time |
| To evaluate the effect of | Change from Baseline in | Mean difference |
| brensocatib compared with | concentration of blood NE, | |
| placebo on the concentration of | CatG, PR3, and neutrophil | |
| biomarkers and neutrophil | functions at Week 24 | |
| functions in blood over 24 weeks | | |
| of treatment | | |
| To evaluate the safety and | Adverse Events (AEs) over 24 | Incidence (%) |
| tolerability of brensocatib | weeks of treatment | |
| compared with placebo over 24 | Clinical laboratory test results, | Mean absolute |
| weeks of treatment | vital signs, and | value and mean |
| | electrocardiograms (ECGs) over | change from |
| | 24 weeks of treatment | Baseline |

Exploratory

| | | |
|---|---|---|
| To evaluate the efficacy of | Change from Baseline to the 28- | Mean difference |
| brensocatib compared with | day average of daily | |
| placebo in improving rhinorrhea | anterior/posterior rhinorrhea | |
| | severity score at Week 24 | |
| To evaluate the efficacy of | Change from Baseline to the 28- | Mean difference |
| brensocatib compared with | day average of daily facial | |
| placebo in improving facial pain | pain/pressure severity score at | |
| | Week 24 | |
| To evaluate the efficacy of | Change from Baseline in | Mean difference |
| brensocatib compared with | University of Pennsylvania | |
| placebo in improving the sense | Smell Identification Test | |
| of smell | (UPSIT) Score at Week 24 | |
| To evaluate the efficacy of | Change from Baseline in | Mean difference |
| brensocatib at 10 and 40 mg QD | modified Lund-Kennedy | |
| compared with placebo in | Endoscopic Score at Week 24 | |
| improving the appearance of the | | |
| nasal mucosa as seen by | | |
| rhinoscopy | | |
| To evaluate the effect of | Responder status for achieving | Percentage of |
| brensocatib compared with | minimal clinically important | responders |
| placebo | difference (MCID) for SNOT-22 | |
| | (≥8.9 points) at Week 24 | |
| To evaluate the use of | Total use of corticosteroids | Median difference |
| corticosteroids throughout the | (mg/day) from Baseline to Week | |
| study period^{b} | 24 | |
| Intercurrent events (ICE) | | |
| Strategy: While-on-treatment for | | |
| nasal surgery | | |
| To evaluate the effect of | Change from Baseline to the 28- | Mean difference |
| brensocatib on the sTSS at Week | day average of daily sTSS at | |
| 24 | Week 24 | |
| Population: Participants with | | |
| CRSsNP who have had prior | | |
| nasal surgery | | |
| Population: CRSsNP participants | | |
| who have not had prior nasal | | |
| surgery | | |
| To evaluate the effect of | Change in PGI-S and PGI-C | Mean difference |
| brensocatib compared with | from Baseline to Weeks 4, 8, 16, | |
| placebo on Patient Global | 20, and 24 | |
| Impression of Severity (PGI-S) | | |
| scale and Patient Global | | |
| Impression of Change (PGI-C) | | |
| scales over the 24-week | | |
| treatment period | | |
| To evaluate effect of brensocatib | Change from Baseline in | Descriptive |
| compared with placebo on the | concentration of active NE, | statistics |
| concentration of biomarkers in | CatG, PR3, and other | |
| nasal secretions over 24 weeks of | biomarkers in nasal secretions at | |
| treatment | Week 24 | |
| To evaluate population PK of | Development of a population | Population PK |
| brensocatib | model to describe brensocatib | model and |
| | PK in participants with CRSsNP | predicted |
| | and predict individual PK | individual PK |
| | parameters, such as Cₘₐₓ, AUC, | parameters |
| | t_{1/2}, CL/F, and V_{d}/F | |
| To explore PK/PD relationships | Relationships between PK | PK-efficacy and |
| on safety, efficacy, and | concentrations and response | PK-safety |
| biomarkers | measures of efficacy (e.g., sTSS, | relationships |
| | etc.), safety (Adverse Events of | |
| | Special Interest (AESIs)), and | |
| | blood NSP activity (NE, CatG, | |
| | PR3) | |
| To evaluate health care | Number of outpatient visits, ER | Mean events per |
| utilization over 24 weeks of | visits, and hospitalizations for | patient year |
| treatment | any reason | |

^{a}Rescue for symptoms of CRS
^{b}Total dose of steroids includes intranasal and systemic steroid use

### Efficacy Assessments

1. sTSS

Participants will self administer the sTSS daily in the eDiary each morning throughout the study until Week 28 (End of Study (EOS)).

Participants will have an sTSS (nasal congestion, anterior/posterior rhinorrhea, facial pain/pressure) ≥5 at Screening and for the weekly average score in the week prior to randomization, which will be used by the Investigator to determine eligibility. For the End of Treatment (EOT) analysis, 4-week average of the symptom scores will be used.

On a daily basis, the participant will use an eDiary to respond to the morning individual rhinosinusitis symptom questions using a 0 to 3 categorical scale (where 0=no symptoms, 1=mild symptoms, 2=moderate symptoms and 3=severe symptoms) to assess symptoms over the previous 24 hours:
Congestion and/or obstruction
Anterior/posterior rhinorrhea (runny nose)
Facial pain/pressure

### 2. Lund-MacKay (LMK) CT Scan Score

The LMK total score is based on assessment of the CT scan findings for each sinus area. The extent of opacification is rated between 0 (normal), 1 (partial opacification), to 2 (total opacification). These points are then applied to the maxillary, anterior ethmoid, posterior ethmoid, sphenoid, and front sinus on each side. The osteomeatal complex is graded as 0 (not occluded) or 2 (occluded). A maximum score of 12 per side is then derived.

A CT scan will be performed anytime during the Screening Period prior to randomization and again at Week 24 (EOT). Participants must have bilateral ethmoid opacification prior to randomization.

### 3. CT Scan Volumetric Assessment

CT scans performed during the Screening Period and at Week 24 will be used for the CT Scan Volumetric assessment.

### 4. SNOT-22

The SNOT-22 is a validated patient-reported outcome (PRO) questionnaire. The questionnaire includes 22 outcome measures on a 5-category scale. The score ranges from 0 to 110 with a higher score indicating greater rhinosinusitis-related health burden.

The SNOT-22 has 22 items on a 5 category scale applicable to sinonasal conditions and surgical treatments. The range of the global score is O to 110 and the minimal clinically important difference (MCID), the smallest difference between clinical study arms mean change from Baseline (point estimates) that will be interpretated as important, is ≥8.9 points. Lower scores indicate less impact and the recall period is the previous 2 weeks. There are 5 domains that can be described within the SNOT-22, including nasal, ear, sleep, general/practical, and emotional.

The SNOT-22 PRO will be completed in the eDiary throughout the study till Week 28 (EOS) to assess symptoms over the past 2 weeks. Participants must have a SNOT-22 score of ≥20 at Screening and Baseline Visits.

5. Nasal congestion score (NCS)

Nasal congestion symptom severity scores are assessed by the participant on a daily basis. Nasal congestion/obstruction symptoms severity over each 24-hour period will be scored by the participant as follows:
0=No symptoms
1=Mild symptoms (symptoms clearly present, but minimal awareness and easily tolerated)
2=Moderate symptoms (definite awareness of symptoms that is bothersome but tolerable)
3=Severe symptoms (symptoms that are hard to tolerate, cause interference with activities or daily living)

NC symptom severity scores will be recorded in the eDiary each morning till Week 28 (EOS) as part of the sTSS. Participants must have ongoing symptoms of NC/obstruction for at least 12 consecutive weeks before Screening and an NCS of≥2 at Screening and for the weekly average score in the week prior to randomization.

### 6. VAS Score

Participants will self administer the VAS electronically to assess symptoms over the past 24 hours. Participants must have a VAS score of ≥5 at Screening and Baseline Visits.

### 7. Peak Nasal Inspiratory Flow (PNIF)

The PNIF will be measured using an in-check portable nasal inspiratory flow meter. At Screening, participants will be provided an PNIF meter to record morning inspiratory flow readings. Participants will be instructed on the use of the device and the Investigator will instruct participants on how to record the following variables in the eDiary on a daily basis:

Morning PNIF is to be performed within 15 minutes of waking (before 12 noon) prior to taking mometasone furoate nasal spray.

Three PNIF readings will be performed by the participant; all 3 values will be recorded by the participants in the eDiary. The highest value of the 3 recorded values will be used for evaluation.

Baseline morning PNIF will be the mean morning measurement recorded for the 7 days prior to the first dose of investigational medicinal product (IMP). The PNIF will be performed daily till Week 28 (EOS).

### 8. PGI-S and PGI-C

The PGI-S and PGI-C scales will be used to assess the participant's overall perception of the severity and change in CRSsNP status. These scales are intended to confirm a meaningful change threshold for the sTSS and other relevant endpoints.

Participants will complete the PGI-S and PGI-C to assess symptoms over the past 2 weeks.

### 9. Proportion of Participants Receiving Rescue Therapy

Participants requiring rescue therapy due to any worsening CRS symptoms will be recorded. Rescue therapy includes antibiotics, steroids, and/or nasal surgery.

### 10. Anterior/Posterior Rhinorrhea Severity Score

The anterior/posterior rhinorrhea severity score is recorded daily as part of the sTSS.

### 11. Daily Facial Pain/Pressure Severity Score

The daily facial pain/pressure severity score is recorded daily as part of the sTSS.

### 12. University of Pennsylvania Smell Identification Test (UPSIT) Score

The UPSIT test is a rapid, easy to administer, 40-item assessment of sense of olfactory function. The UPSIT shows a high test-retest reliability (r: 0.981) and scores on this test are strongly correlated with the detection threshold for phenyl ethyl alcohol in the same individuals. When the UPSIT is administered in the standardized manner, clinical participants show a high degree of uniformity in UPSIT performance when tested in different laboratories. A particular strength of this test is that it provides an olfactory diagnosis based on comparing the patient's test score with normative data, providing a percentile score of an individual relative to his or her age-matched normal group. The UPSIT test score can distinguish patients with a normal sense of smell (“normosomia”) from those with reduced sense of smell (“mild, moderate, and severe microsmia”) or total loss of sense of smell (“anosmia”).

The test consists of 4 booklets, each containing 10 odorants. The stimuli are embedded in 10-50 (mu) diameter plastic microcapsules on brown strips at the bottom of each page. The participant releases the odorant by rubbing the embedded odorants. Above each odorant is a multiple-choice question with 4 words to describe the odor. The odorants of the UPSIT test utilized in this study will take into account cultural differences. Participants will receive a score out of 40 possible correct answers.

### 13. Modified Lund-Kennedy Endoscopic Score

The Lund-Kennedy endoscopy scoring system grades visual pathologic states within the nose and paranasal sinuses including polyps, discharge, edema, scarring, and crusting. The modified LK system excluding subscores of scarring and crusting improves its reliability with patient reported outcomes. See Psaltis et al., Laryngoscope 124(10):2216-23 (2014), incorporated herein by reference in its entirety.

The modified Lund-Kennedy grading system is as follows:

| Characteristic | Score* | Definition |
|---|---|---|
| Nasal Polyps | 0 | no polyps |
| | 1 | polyps in middle meatus only |
| | 2 | beyond middle meatus |
| Discharge | 0 | no discharge |
| | 1 | clear, thin discharge |
| | 2 | thick, purulent discharge |
| Edema | 0 | absent |
| | 1 | mild |
| | 2 | severe |

*Higher score indicates worse sinus disease

For endpoint analysis, the endoscopy reading at Screening will be compared to the Week 24 reading.

### 14. Proportion of Participants Requiring Nasal Surgery

Participants requiring nasal surgery due to any worsening CRS symptoms will be recorded, including the type of surgery, reason for surgery, date of surgery, and outcome of surgery.

### Pharmacokinetic Assessments

Blood samples of approximately 6 mL each will be collected for measurement of plasma concentrations of brensocatib and for PK analysis. The timepoints for PK sample collections include: pre-dose, 1 h (±5 min), 2 h (±10 min), and 6 h (±2 h) on Day 1; post-dose at Week 4; pre-dose at Week 8; post-dose at Week 12; pre-dose at Week 16; post-dose at Week 20; and pre-dose at Week 24.

### Pharmacodynamic Assessments

### 1. PD Biomarkers

Blood samples and nasal secretions will be collected to evaluate the effect of brensocatib compared with placebo on NSP and NET activity. Biomarkers will include NE, CatG, PR3 and other relevant biomarkers. Samples will be collected at the following time points: Day 1; and Weeks 4, 8, 12, 16, 20, 24, and 28.

Changes from Baseline will be explored for the inhibitory effect of brensocatib on the biomarkers.

### 2. PD Substudy

A PD substudy will be conducted in a subset of participants. Nasal secretion samples will be collected from approximately 60 participants: 30 participants from the population with blood eosinophil count <300 cells/μL and 30 participants from the population with blood eosinophil count ≥300 cells/μL at Screening. Samples will be collected at in-clinic visits at Day 1, Week 4, and Week 24.

A Sinus Pack will be inserted bilaterally into the nasal cavities for 5 minutes. The two packings will be transferred to a tube, and the adsorbed analytes eluted from the packings by adding 3 mL of saline (0.9% NaCl) followed by centrifugation. The supernatant aliquots will be stored at −70° C.

### Population PK and PK/PD Evaluations

Plasma concentration data from this study will be utilized in population PK analysis as an exploratory measure. The individual PK parameters, such as Cₘₐₓ, Tₘₐₓ, AUC₀₋₂₄, elimination t_{1/2}, CL/F, and V_{d}/F, will be determined using a population PK model.

Based on population PK model-predicted systemic exposure, PK-efficacy, and PK-safety relationships, such as the response measures of efficacy (e.g., sTSS, etc.), safety (AESIs), and blood NSP activity (NE, CatG, PR3), will be evaluated. The identified PK-PD relationship will be used to predict clinical outcome via Monte Carlo simulation to support the dose and dosing regimen selection for future clinical studies.

The population attribute for the primary efficacy estimand is participants with CRSsNP who have baseline eosinophil counts <300 cells/μL. The entire population of participants with CRSsNP will be secondary for the efficacy estimands and primary for the safety, pharmacokinetic, and pharmacodynamic estimands. For all estimands, the treatment regimen is the randomized study treatment (i.e., brensocatib 10 mg QD, brensocatib 40 mg QD, or placebo QD).

### Safetv Parameters

Safety parameters will include AEs, clinical laboratory test results, vital sign measurements, ECG measurements, and physical examination results. Adverse events of special interest (AESIs) will include hyperkeratosis, periodontitis/gingivitis, and severe infection.

Data Analyses Analvsis Sets

Screened Analysis Set—Includes all participants who provide written informed consent.

Full Analysis Set—Includes all randomized participants. Participants are analyzed according to their assigned treatment.

Safety Analysis Set—Includes all randomized participants who receive at least 1 dose of study treatment. Participants will be analyzed according to the treatment received.

PK Concentration Analysis Set—Includes all participants who receive at least 1 dose of brensocatib and have at least 1 post-dose concentration value.

PD Analysis Set—Includes all participants who receive at least 1 dose of their assigned study treatment and have at least 1 pre-dose and 1 post-dose measurement of biomarkers.

### Efficacy Analyses

The efficacy estimands will utilize a composite strategy for handling of intercurrent events (ICEs) related to rescue (i.e., systemic corticosteroids, antibiotics, or nasal surgery). Data after the ICE of nasal surgery will be imputed using the worst possible score and data after the ICE of rescue medication (systemic corticosteroids or antibiotics) will be imputed using worst observation carried forward (WOCF). Early discontinuation of treatment without an ICE related to rescue will be handled using the treatment policy strategy.

The primary efficacy endpoint is the change from Baseline to the 28-day average of daily sTSS at Week 24 in participants with CRSsNP who have a baseline eosinophil count <300 cells/μL and will be analyzed using an analysis of covariance (ANCOVA) model with a main effect for treatment, a continuous covariate for Baseline mean daily value, and the randomization stratification factors for history of prior surgery for CRSsNP (Yes/No), geography, and current diagnosis of asthma (Yes/No). The least squares mean difference, the two-sided 95% confidence intervals (CIs), and the corresponding p-values for brensocatib 10 mg vs placebo and brensocatib 40 mg vs placebo will be summarized.

The secondary analysis of the primary endpoint will be in the overall population of participants with CRSsNP and will be analyzed with a similar ANCOVA model. However, a continuous covariate for baseline blood eosinophil count will be added. In addition, a check for confounding between asthma diagnosis and blood eosinophil count will be performed.

For the secondary variables that are assessed at Week 24 (i.e., change from Baseline in LMK CT score, change from Baseline in percentage sinus opacification as measured by volumetry, and change from Baseline in the SNOT-22, change from Baseline to the 28-day average of daily NCS, change from Baseline to the 28-day average of daily VAS scores, and change from Baseline to the 28-day average of daily PNIF) separate ANCOVA models will be fit to the data for each variable. The models for the primary population will contain a main effect for treatment, a continuous covariate for the corresponding Baseline value, and the randomization stratification factors for history of prior surgery for CRSsNP (Yes/No), geography, and current diagnosis of asthma. The models for analyses in the overall population will also contain a continuous covariate for baseline blood eosinophil count. The least squares mean differences, 95% CIs, and the corresponding p-values for brensocatib 10 mg vs placebo and brensocatib 40 mg vs placebo will be summarized.

The time-to-first use of rescue (systemic corticosteroids, antibiotics, or nasal surgery) will be compared among treatments using Cox proportional hazards regression. The estimated hazard ratios with corresponding 95% confidence limits and two-sided Wald p-values from the Cox model will be reported for each comparison. Kaplan-Meier plots will summarize the probabilities of each rescue event (systemic corticosteroids, antibiotics, or nasal surgery) by treatment over time.

The proportion of participants requiring rescue (systemic corticosteroids, antibiotics, or nasal surgery) will be compared among treatments using logistic regression. The estimated odds ratios with corresponding 95% confidence limits and two-sided Wald p-values from a logistic regression model will be reported for comparisons of brensocatib 10 mg vs placebo and of brensocatib 40 mg vs placebo.

### Pharmacokinetic Analyses

Plasma concentrations of brensocatib will be listed and summarized by dose level over each scheduled sampling time point using descriptive statistics. Individual plasma concentration data versus time will be listed, along with graphical plots of individual and mean plasma concentration-time plots presented in linear and semi-logarithmic scale.

### Pharmacodynamic Analyses

NSP activity in blood and nasal secretions (NE, CatG, and PR3) will be assessed as percent inhibition from baseline. Data will be listed and summarized by treatment group over each scheduled sampling time point using descriptive statistics. Model-based analyses may be conducted if sample sizes permit. Other biomarkers may be assessed.

### Safety Analyses

Safety analyses will assess frequency and severity of treatment-emergent adverse events, including SAEs and AESIs, changes from Baseline in clinical laboratory test results, and vital signs, and ECG. All safety analyses will be performed by treatment received on the Safety Analysis Set.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method of inhibiting dipeptidyl peptidase (DPP1) in a subject having chronic rhinosinusitis (CRS) or at risk of developing CRS, comprising,
administering to the subject for an administration period, a pharmaceutical composition comprising an effective amount of a compound of Formula (I), or a pharmaceutically acceptable salt thereof, 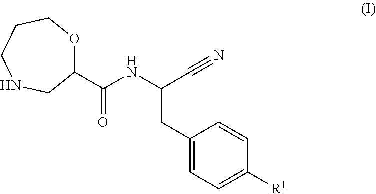
wherein,
R¹ is 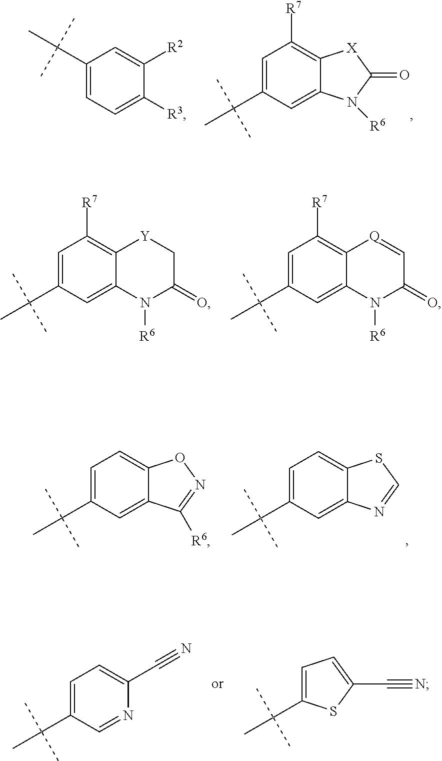
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring; or
X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃,
thereby inhibiting DPP1 in the subject.

2. The method of claim 1, wherein the subject is at a risk for developing CRS.

3. The method of claim 2, wherein the subject at a risk for developing CRS has or had one or more of the following: acute rhinosinusitis, viral respiratory tract infection, allergic rhinitis, nonallergic rhinitis, asthma, bronchitis, pneumonia, gastroesophageal reflux disease, adenotonsillitis, sleep apnea, otitis media, allergic or nonallergic upper airway disease, allergic or nonallergic lower airway disease, epithelial cell disorder, common variable immunodeficiency, HIV infection, cystic fibrosis (CF), ciliary dyskinesia, Wegener granulomatosis, sarcoidosis, and chronic obstructive pulmonary disease.

4. The method of claim 2 or 3, wherein the subject is or was repeatedly exposed to tobacco smoke.

5. A method of treating chronic rhinosinusitis (CRS) in a subject in need thereof, comprising,
administering to the subject for an administration period, a pharmaceutical composition comprising an effective amount of a compound of Formula (I), or a pharmaceutically acceptable salt thereof, 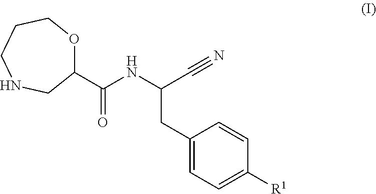
wherein,
R¹ is 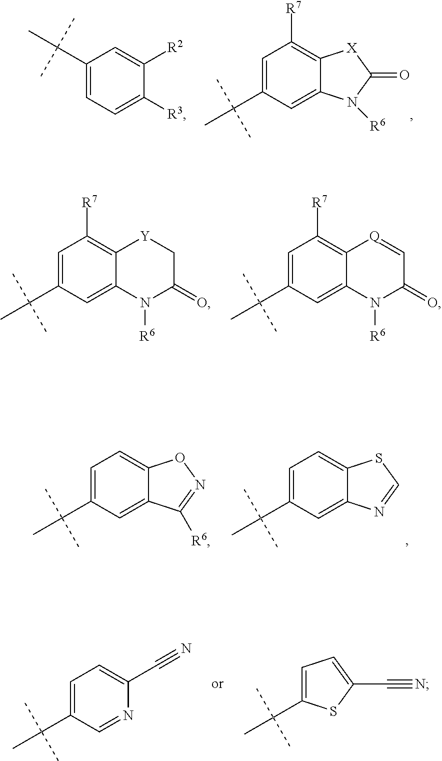
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring; or
X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃,
thereby treating chronic rhinosinusitis in the subject.

6. The method of any one of claims 1-5, wherein the CRS is associated with the presence or development of one or more conditions selected from the group consisting of allergic conjunctivitis, atopic dermatitis, asthma, a urinary tract infection, a skin infection and a soft tissue infection.

7. The method of any one of claims 1-6, wherein the subject is an adult with CRS who has two or more CRS symptoms, wherein one of the symptoms is nasal blockage, nasal obstruction, nasal congestion, or nasal discharge.

8. The method of claim 7, wherein the two or more CRS symptoms further comprise facial pain or pressure.

9. The method of claim 7, wherein the two or more CRS symptoms do not comprise facial pain or pressure.

10. The method of any one of claims 7-9, wherein the two or more CRS symptoms further comprise reduction or loss of smell.

11. The method of any one of claims 7-9, wherein the two or more CRS symptoms do not comprise reduction or loss of smell.

12. The method of any one of claims 7-11, wherein the nasal blockage, nasal obstruction, nasal congestion, or nasal discharge is present for ≥12 weeks.

13. The method of any one of claims 7-12, wherein the nasal discharge comprises anterior nasal drip or posterior nasal drip.

14. The method of any one of claims 1-6, wherein the subject is a child with CRS who has two or more symptoms, wherein one of the symptoms is nasal blockage, nasal obstruction, nasal congestion, or nasal discharge.

15. The method of claim 14, wherein the two or more CRS symptoms further comprise facial pain or pressure.

16. The method of claim 14, wherein the two or more CRS symptoms do not comprise facial pain or pressure.

17. The method of any one of claims 14-16, wherein the two or more CRS symptoms further comprise cough.

18. The method of any one of claims 14-16, wherein the two or more CRS symptoms do not comprise cough.

19. The method of any one of claims 14-18, wherein the nasal blockage, nasal obstruction, nasal congestion, or nasal discharge is present for ≥12 weeks.

20. The method of any one of claims 14-19, wherein the nasal discharge comprises anterior nasal drip or posterior nasal drip.

21. The method of any one of claims 1-20, wherein the subject has difficult-to-treat CRS.

22. The method of any one of claims 1-21, wherein the CRS is CRS without nasal polyps (CRSsNP), or CRS with nasal polyps (CRSwNP).

23. The method of claim 22, wherein the CRS is CRSsNP.

24. The method of claim 23, wherein the subject is a CRSsNP patient with an eosinophil count <300 cells/μL prior to the administration period.

25. The method of claim 22, wherein the CRS is CRSwNP.

26. The method of any one of claims 1-25, wherein the CRS is refractory CRS.

27. The method of claim 26, wherein the CRS is steroid-refractory.

28. The method of claim 26 or 27, wherein the refractory CRS is refractory CRSsNP.

29. The method of claim 26 or 27, wherein the refractory CRS is refractory CRSwNP.

30. The method of claim 5, wherein the subject is at risk for developing CRS and the treating comprises providing prophylaxis against CRS.

31. The method of any one of claims 1-29, further comprising decreasing the severity of one or more symptoms of CRS of the subject, during or subsequent to the administration period, compared to the one or more symptoms of CRS prior to the administration period.

32. The method of any one of claims 1-31, wherein during or subsequent to the administration period, the onset of one or more symptoms of CRS is delayed.

33. The method of claim 31 or 32, wherein the one or more symptoms of CRS are: nasal congestion; nasal obstruction; nasal discharge; post-nasal drip; facial pressure; facial pain; facial fullness; reduced smell; depression; mucosal edema; mucopurulent discharge; obstruction of the middle meatus; mucosal changes within the ostiomeatal complex and sinuses; rhinorrhea; or any combinations thereof.

34. The method of claim 33, wherein the rhinorrhea is anterior rhinorrhea.

35. The method of claim 33, wherein the rhinorrhea is posterior rhinorrhea.

36. The method of any one of claims 1-35, wherein the method comprises decreasing a Lund-Mackay score of the subject during or subsequent to the administration period as compared to a Lund-Mackay score of the subject prior to the administration period.

37. The method of claim 36, wherein the Lund-Mackay score of the subject during or subsequent to the administration period is less than 4.

38. The method of claim 36 or 37, wherein the method comprises decreasing the Lund-Mackay score of the subject during or subsequent to the administration period by about 5%, about 10%, about 20%, about 30%, about 40%, about 50%, about 60%, about 70%, about 80%, about 90%, or about 100%, as compared to the Lund-Mackay score of the subject prior to the administration period.

39. The method of any one of claims 36-38, wherein the method comprises decreasing the Lund-Mackay score of the subject during or subsequent to the administration period by 1, 2, 3, 4, 5, 6, 7, 8, 9, 10, 11, 12, 13, 14, 1,5 16, 17, 18, 19, 20, 21, 22, 23, or 24, as compared to the Lund-Mackay score of the subject prior to the administration period.

40. The method of any one of claims 36-38, wherein the method comprises decreasing the Lund-Mackay score of the subject during or subsequent to the administration period by from 1 to 24, from 1 to 20, from 1 to 16, from 1 to 12, from 1 to 8 or from 1 to 4, as compared to the Lund-Mackay score of the subject prior to the administration period.

41. The method of any one of claims 36-38, wherein the method comprises decreasing the Lund-Mackay score of the subject during or subsequent to the administration period by from 4 to 24, from 8 to 24, from 12 to 24, from 16 to 24, or from 20 to 24, as compared to the Lund-Mackay score of the subject prior to the administration period.

42. The method of any one of claims 36-41, wherein the Lund-Mackay score prior to the administration period and the Lund-Mackay score during or subsequent to the administration period are calculated based on computed tomography (CT) scans of the subject.

43. The method of claim 42, wherein the CT scans are performed on the paranasal sinuses of the subject, one or both of ostiomeatal complexes of the subject, or a combination thereof.

44. The method of claim 43, wherein the CT scans are performed on the right ostiomeatal complex of the subject, the left ostiomeatal complex of the subject, or a combination thereof.

45. The method of claim 43, wherein the CT scans are performed on one or more of right frontal sinuses; left frontal sinuses; right anterior ethmoidal sinuses; left anterior ethmoidal sinuses; right posterior ethmoidal sinuses; left posterior ethmoidal sinuses; right maxillary sinuses; left maxillary sinuses; right sphenoid sinuses of the subject; left sphenoid sinuses of the subject; or combinations thereof.

46. The method of any one of claims 36-45, wherein the Lund-Mackay score of the subject prior to the administration period is greater than or equal to 4.

47. The method of any one of claims 1-46, wherein the method comprises decreasing a rhinoscopy sum score of the subject during or subsequent to the administration period, as compared to a rhinoscopy sum score of the subject prior to the administration period.

48. The method of claim 47, wherein the rhinoscopy sum score of the subject prior to the administration period is greater than a rhinoscopy sum score of a control subject who does not have CRS.

49. The method of claim 47 or 48, wherein the rhinoscopy sum score of the subject prior to the administration period is greater than 1.

50. The method of any one of claims 47-49, wherein the rhinoscopy sum score of the subject during or subsequent to the administration period is less than or equal to 1.

51. The method of any one of claims 1-50, wherein the method comprises decreasing a Sino-Nasal Outcome Test-22 (SNOT-22) score of the subject during or subsequent to the administration period, as compared to a SNOT-22 score of the subject prior to the administration period.

52. The method of claim 51, wherein the SNOT-22 score of the subject prior to the administration period is higher than a SNOT-22 score of a control subject who does not have CRS.

53. The method of claim 51 or 52, wherein prior to the administration period, the SNOT-22 score of the subject is greater than or equal to 20.

54. The method of any one of claims 51-53, wherein prior to the administration period, the SNOT-22 score of the subject is greater than or equal to 30.

55. The method of any one of claims 51-54, wherein the SNOT-22 score of the subject during or subsequent to the administration period is less than 30.

56. The method of any one of claims 51-55, wherein the SNOT-22 score of the subject during or subsequent to the administration period is less than 20.

57. The method of any one of claims 51-56, wherein the method comprises decreasing the SNOT-22 score of the subject during or subsequent to the administration period by about 5%, about 10%, about 20%, about 30%, about 40%, about 50%, about 60%, about 70%, about 80%, about 90%, or about 100%, as compared to the SNOT-22 score of the subject prior to the administration period.

58. The method of any one of claims 51-57, wherein the method comprises decreasing the SNOT-22 score of the subject during or subsequent to the administration period by 8 points or more, 9 points or more, or 10 points or more, as compared to the SNOT-22 score of the subject prior to the administration period.

59. The method of any one of claims 51-58, wherein the method comprises decreasing the SNOT-22 score of the subject during or subsequent to the administration period by from about 8 to about 20 points, from about 8 to about 18 points, from about 8 to about 16 points or from about 8 to about 14 points, as compared to the SNOT-22 score of the subject prior to the administration period.

60. The method of any one of claims 1-59, comprising increasing a University of Pennsylvania Smell Identification Test (UPSIT) score of the subject during or subsequent to the administration period, as compared to a UPSIT score of the subject prior to the administration period.

61. The method of claim 60, wherein the UPSIT score of the subject prior to the administration period is less than a UPSIT score of a control subject who does not have CRS.

62. The method of claim 60 or 61, wherein the UPSIT score of the subject prior to the administration period is less than 33.

63. The method of any one of claims 60-62, wherein the UPSIT score of the subject during or subsequent to the administration period is greater than 33.

64. The method of any one of claims 60-62, wherein the UPSIT score of the subject during or subsequent to the administration period is greater than 18.

65. The method of any one of claims 60-64, wherein the method comprises increasing the UPSIT score of the subject during or subsequent to the administration period by at least about 5%, at least about 10%, at least about 20%, at least about 30%, at least about 40%, at least about 50%, at least about 60%, at least about 70%, at least about 80%, at least about 90%, as compared to the UPSIT score of the subject prior to the administration period.

66. The method of any one of claims 60-65, wherein the method comprises increasing the UPSIT score of the subject during or subsequent to the administration period by 1, 2, 3, 4, 5, 6,7,8,9, 10, 11, 12, 13, 14, 15 16, 17, 18, 19, 20, 21, 22, 23, 24, 25, 26, 27, 28, 29, 30, 31, 32, 33, 34, 35, 36, 37, 38, 39, or 40, as compared to the UPSIT score of the subject prior to the administration period.

67. The method of any one of claims 60-65, wherein the method comprises increasing the UPSIT score of the subject during or subsequent to the administration period by from about 5 to about 40, from about 10 to about 40, from about 15 to about 40, from about 20 to about 40, from about 25 to about 40, from about 30 to about 40 or from about 35 to about 40, as compared to the UPSIT score of the subject prior to the administration period.

68. The method of any one of claims 60-65, wherein the method comprises increasing the UPSIT score of the subject during or subsequent to the administration period by from about 1 to about 5, from about 5 to about 10, from about 15 to about 20, from about 5 to about 15, from about 5 to about 20, from about 10 to about 20 or from about 10 to about 25, as compared to the UPSIT score of the subject prior to the administration period.

69. The method of any one of claims 1-68, comprising decreasing a modified Lund-Kennedy (MLK) score of the subject during or subsequent to the administration period, as compared to a MLK score of the subject prior to the administration period.

70. The method of claim 69, wherein the MLK score of the subject prior to the administration period is greater than or equal to 4.

71. The method of claim 69 or 70, wherein the MLK score of the subject prior to the administration period is greater than an MLK score of a control subject who does not have CRS.

72. The method of any one of claims 69-71, wherein the MLK score of the subject during or subsequent to the administration period is less than 4.

73. The method of any one of claims 69-72, wherein the method comprises decreasing the MLK score of the subject during or subsequent to the administration period by about 5%, about 10%, about 20%, about 30%, about 40%, about 50%, about 60%, about 70%, about 80%, about 90%, or about 100%, as compared to the MLK score of the subject prior to the administration period.

74. The method of any one of claims 69-73, wherein the method comprises decreasing the MLK score of the subject during or subsequent to the administration period by 1, 2, 3, 4, 5, 6, 7, 8, 9, 10, 11, or 12, as compared to the MLK score of the subject prior to the administration period.

75. The method of any one of claims 69-73, wherein the method comprises decreasing the MLK score of the subject during or subsequent to the administration period from about 2 to about 12, from about 2 to about 10, from about 2 to about 8, from about 2 to about 6 or from about 2 to about 4, as compared to the MILK score of the subject prior to the administration period.

76. The method of any one of claims 1-75, wherein the method further comprises enhancing sinus drainage of the subject during or subsequent to the administration period, as compared to sinus drainage of the subject prior to the administration period.

77. The method of any one of claims 1-75, comprising decreasing a composite severity score of two or more CRS symptoms of the subject during or subsequent to the administration period, as compared to a composite severity score of the subject prior to the administration period.

78. The method of claim 77, wherein the method comprises decreasing the composite severity score of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the composite severity score of the subject prior to the administration period.

79. The method of claim 78, comprising decreasing the composite severity score of the subject during or subsequent to the administration period by at least about 10%.

80. The method of claim 79, comprising decreasing the composite severity score of the subject during or subsequent to the administration period by from about 10% to about 60%, from about 10% to about 50%, from about 10% to about 40%, from about 10% to about 30% or from about 10% to about 20%.

81. The method of any one of claims 77-80, comprising decreasing the composite severity score of the subject during or subsequent to the administration period by about 0.5, about 1, about 1.5, about 2, about 2.5, about 3, about 3.5, about 4, about 4.5, about 5, about 5.5, about 6, about 6.5, about 7, about 7.5, about 8, about 8.5, or about 9 points, as compared to the composite severity score of the subject prior to the administration period.

82. The method of any one of claims 77-80, comprising decreasing the composite severity score of the subject during or subsequent to the administration period by from about 1 to about 9 points, from about 2 to about 9 points, from about 3 to about 9 points, from about 4 to about 9 points, from about 5 to about 9 point, from about 6 to about 9 points, from about 2 to about 7 points, from about 2 to about 6 points or from about 2 to about 5 points, as compared to the composite severity score of the subject prior to the administration period.

83. The method of any one of claims 1-82, further comprising decreasing a sinus total symptom score (sTSS) of the subject during or subsequent to the administration period, as compared to a sTSS of the subject prior to the administration period.

84. The method of claim 83, wherein the subject has an sTSS of ≥5 prior to the administration period.

85. The method of claim 83 or 84, wherein the method comprises decreasing the sTSS of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the sTSS of the subject prior to the administration period.

86. The method of claim 85, comprising decreasing the sTSS of the subject during or subsequent to the administration period by at least about 10%.

87. The method of claim 86, comprising decreasing the sTSS of the subject during or subsequent to the administration period by from about 10% to about 60%, from about 10% to about 50%, from about 10% to about 40%, from about 10% to about 30% or from about 10% to about 20%.

88. The method of any one of claims 83-87, comprising decreasing the sTSS of the subject during or subsequent to the administration period by about 0.5, about 1, about 1.5, about 2, about 2.5, about 3, about 3.5, about 4, about 4.5, about 5, about 5.5, about 6, about 6.5, about 7, about 7.5, about 8, about 8.5, or about 9 points, as compared to the sTSS of the subject prior to the administration period.

89. The method of any one of claims 83-87, comprising decreasing the sTSS of the subject during or subsequent to the administration period by from about 1 to about 9 points, from about 2 to about 9 points, from about 3 to about 9 points, from about 4 to about 9 points, from about 5 to about 9 point, from about 6 to about 9 points, from about 2 to about 7 points, from about 2 to about 6 points or from about 2 to about 5 points, as compared to the sTSS of the subject prior to the administration period.

90. The method of any one of claims 1-89, comprising decreasing a nasal congestion score (NCS) of the subject during or subsequent to the administration period, as compared to an NCS of the subject prior to the administration period.

91. The method of claim 90, wherein the NCS of the subject prior to the administration period is ≥2.

92. The method of claim 90 or 91, comprising decreasing the NCS of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the NCS of the subject prior to the administration period.

93. The method of claim 92, comprising decreasing the NCS of the subject during or subsequent to the administration period by at least about 10%.

94. The method of claim 93, comprising decreasing the NCS of the subject during or subsequent to the administration period by from about 10% to about 60%, from about 10% to about 50%, from about 10% to about 40%, from about 10% to about 30% or from about 10% to about 20%.

95. The method of any one of claims 90-94, comprising decreasing the NCS of the subject during or subsequent to the administration period by about 0.5, about 1, about 1.5, about 2, about 2.5, or about 3 points, as compared to the NCS of the subject prior to the administration period.

96. The method of any one of claims 1-95, comprising decreasing an anterior/posterior rhinorrhea severity score of the subject during or subsequent to the administration period, as compared to an anterior/posterior rhinorrhea severity score of the subject prior to the administration period.

97. The method of claim 96, comprising decreasing the anterior/posterior rhinorrhea severity score of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the anterior/posterior rhinorrhea severity score of the subject prior to the administration period.

98. The method of claim 97, wherein the method comprises decreasing the anterior/posterior rhinorrhea severity score of the subject during the administration period or subsequent to the administration period by at least about 10%.

99. The method of claim 98, comprising decreasing the anterior/posterior rhinorrhea severity score of the subject during or subsequent to the administration period by from about 10% to about 60%, from about 10% to about 50%, from about 10% to about 40%, from about 10% to about 30% or from about 10% to about 20%.

100. The method of any one of claims 96-99, wherein the method comprises decreasing the anterior/posterior rhinorrhea severity score of the subject during or subsequent to the administration period by about 0.5, about 1, about 1.5, about 2, about 2.5, or about 3 points, as compared to the anterior/posterior rhinorrhea severity score of the subject prior to the administration period.

101. The method of any one of claims 1-100, comprising decreasing a facial pain/pressure severity score of the subject during or subsequent to the administration period, as compared to a facial pain/pressure severity score of the subject prior to the administration period.

102. The method of claim 101, comprising decreasing the facial pain/pressure severity score of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the facial pain/pressure severity score of the subject prior to the administration period.

103. The method of claim 102, comprising decreasing the facial pain/pressure severity score of the subject during or subsequent to the administration period by at least about 10%.

104. The method of claim 103, comprising decreasing the facial pain/pressure severity score of the subject during or subsequent to the administration period by from about 10% to about 60%, from about 10% to about 50%, from about 10% to about 40%, from about 10% to about 30% or from about 10% to about 20%.

105. The method of any one of claims 101-104, comprising decreasing the facial pain/pressure severity score of the subject during or subsequent to the administration period by about 0.5, about 1, about 1.5, about 2, about 2.5, or about 3 points, as compared to the facial pain/pressure severity score of the subject prior to the administration period.

106. The method of any one of claims 1-105, comprising decreasing a Visual Analog Scale (VAS) score of the subject during or subsequent to the administration period, as compared to a VAS score of the subject prior to the administration period.

107. The method of claim 106, comprising decreasing the VAS score of the subject from a severe score prior to the administration period to a moderate score during or subsequent to the administration period.

108. The method of claim 106, comprising decreasing the VAS score of the subject from a severe score prior to the administration period to a mild score of during or subsequent to the administration period.

109. The method of claim 106, comprising decreasing the VAS score of the subject from a moderate score prior to the administration period to a mild score during or subsequent to the administration period.

110. The method of any one of claims 106-109, wherein the VAS score of the subject is ≥5 prior to the administration period.

111. The method of any one of claims 106-110, comprising decreasing the VAS score of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the VAS score of the subject prior to the administration period.

112. The method of claim 111, comprising decreasing the VAS score of the subject during or subsequent to the administration period by at least about 10%.

113. The method of claim 112, comprising decreasing the VAS score of the subject during or subsequent to the administration period by from about 10% to about 60%, from about 10% to about 50%, from about 10% to about 40%, from about 10% to about 30% or from about 10% to about 20%.

114. The method of any one of claims 1-113, comprising increasing a Peak Nasal Inspiratory Flow (PNIF) of the subject during or subsequent to the administration period, as compared to a PNIF of the subject prior to the administration period.

115. The method of claim 114, comprising increasing the PNIF of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the PNIF of the subject prior to the administration period.

116. The method of claim 115, comprising increasing the PNIF of the subject during or subsequent to the administration period by at least about 10%.

117. The method of claim 116, comprising increasing the PNIF of the subject during or subsequent to the administration period by from about 10% to about 60%, from about 10% to about 50%, from about 10% to about 40%, from about 10% to about 30% or from about 10% to about 20%.

118. The method of any one of claims 1-117, comprising decreasing a percentage of sinus opacification of the subject as measured by CT scan volumetry during or subsequent to the administration period as compared to a percentage of sinus opacification of the subject prior to the administration period.

119. The method of any one of claims 1-118, comprising improving a Patient Global Impression of Severity (PGI-S) score or a Patient Global Impression of Change (PGI-C) score of the subject during or subsequent to the administration period, as compared to a PGI-S score or a PGI-C score of the subject prior to the administration period.

120. The method of claim 119, comprising improving the PGI-S score or the PGI-C score of the subject during or subsequent to the administration period by at least about 5%, at least about 10%, at least about 20%, at least about 30%, at least about 40%, at least about 50%, at least about 60%, at least about 70%, at least about 80%, or at least about 90%, as compared to the PGI-S score or the PGI-C score of the subject prior to the administration period.

121. The method of claim 120, comprising improving the PGI-S score or the PGI-C score of the subject during or subsequent to the administration period by at least about 10%.

122. The method of claim 121, comprising improving the PGI-S score or the PGI-C score of the subject during or subsequent to the administration period by from about 10% to about 60%, from about 10% to about 50%, from about 10% to about 40%, from about 10% to about 30% or from about 10% to about 20%.

123. The method of any one of claims 119-122, comprising improving the PGI-S score of the subject during or subsequent to the administration period by about 0.5, about 1, about 1.5, about 2, about 2.5, about 3, about 3.5, about 4, or about 4.5 points, as compared to the PGI-S score of the subject prior to the administration period.

124. The method of any one of claims 119-123, comprising improving the PGI-C score of the subject during or subsequent to the administration period by about 0.5, about 1, about 1.5, about 2, about 2.5, about 3, about 3.5, about 4, about 4.5 points, about 5, about 5.5, about 6, or about 6.5 points, as compared to the PGI-C score of the subject prior to the administration period.

125. The method of any one of claim 1-124, comprising increasing the length of time to first use of rescue with a systemic corticosteroid, an antibiotic, or nasal surgery of the subject, as compared to a control subject, wherein the control subject has CRS and is not administered the pharmaceutical composition.

126. The method of any one of claim 1-125, comprising reducing the frequency of rescue with a systemic corticosteroid, an antibiotic, or nasal surgery of the subject due to worsening of CRS symptoms, as compared to a control subject, wherein the control subject has CRS and is not administered the pharmaceutical composition.

127. The method of any one of claims 1-126, wherein the number of neutrophils in a biological sample obtained from the subject prior to the administration period is greater than the number of neutrophils in a counterpart biological sample obtained from a control subject who does not have CRS.

128. The method of any one of claims 1-127, wherein the level of ICAM1 in a biological sample obtained from the subject prior to the administration period is greater than the level of ICAM1 in a counterpart biological sample obtained from a control subject who does not have CRS.

129. The method of any one of claims 1-128, wherein prior to the administration period, the level of DPP1 in a biological sample obtained from the subject is in the range of about 1 ng/mL to about 100 ng/mL.

130. The method of any one of claims 1-129, wherein the activity of DPP1 in a biological sample obtained from the subject during or subsequent to the administration period is less than: (a) the activity of DPP1 in a counterpart biological sample obtained from the subject prior to the administration period, and/or (b) the activity of DPP1 in a counterpart biological sample obtained from a control subject, wherein the control subject has CRS and is not administered the pharmaceutical composition.

131. The method of any one of claims 1-130, wherein prior to the administration period, the level of a neutrophil serine protease (NSP) in a biological sample obtained from the subject is in the range of about 1 ng/mL to about 1000 ng/mL.

132. The method of any one of claims 1-131, wherein the activity of a neutrophil serine protease (NSP) in a biological sample obtained from the subject during or subsequent to the administration period is less than: (a) the activity of the NSP in a counterpart biological sample obtained from the subject prior to the administration period, and/or (b) the activity of the NSP in a counterpart biological sample obtained from a control subject, wherein the control subject has CRS and is not administered the pharmaceutical composition.

133. The method of claim 131 or 132, wherein the NSP is neutrophil elastase (NE), proteinase 3 (PR3), cathepsin G (CatG), neutrophil serine protease 4 (NSP4), or any combination thereof.

134. The method of any one of claims 1-133, wherein prior to the administration period, the level of neutrophil extracellular traps (NETs) in a biological sample obtained from the subject is in the range of about 1 ng/mL to about 1000 ng/mL.

135. The method of any one of claims 1-134, wherein the level of neutrophil extracellular traps (NETs) in a biological sample obtained from the subject during or subsequent to the administration period is less than: (a) the level of NETs in a counterpart biological sample obtained from the subject prior to the administration period, and/or (b) the level of NETs in a counterpart biological sample obtained from a control subject, wherein the control subject has CRS and is not administered the pharmaceutical composition.

136. The method of any one of claims 127-135, wherein the biological sample comprises blood, sinonasal tissue, or a combination thereof.

137. The method of claim 136, wherein the biological sample comprises blood.

138. The method of claim 136, wherein the biological sample comprises sinonasal tissue.

139. The method of claim 136, wherein the biological sample comprises a combination of blood and sinonasal tissue.

140. The method of any one of claims 1-139, wherein DPP1 is expressed by neutrophils.

141. The method of any one of claims 1-140, wherein the subject has one or more mutations in a gene encoding a protein selected from the group consisting of: Ring1A and YY1 binding protein (RYBP), acyloxyacyl hydrolase (AOAH), IL-1 receptor-associated kinase 4, IL-1 receptor-like 1, Toll-like receptor (TLR)-2, TLR-1, TLR-5, cystic fibrosis transmembrane conductance regulator (CFTR), and transforming growth factor beta-1.

142. The method of claim 141, wherein the one or more mutations is a single nucleotide polymorphism.

143. The method of claim 142, wherein the single nucleotide polymorphism is rs4504543 in the gene encoding AOAH, or rs4532099 in the gene encoding RYBP.

144. The method of any one of claims 1-143, further comprising administering a secondary therapy to the subject.

145. The method of claim 144, wherein the secondary therapy comprises one or more of the following: a steroid, an antihistamine, an antibiotic, an anti-depressant, a biologic, an anti-leukotriene, nasal saline irrigation, and a surgical intervention.

146. The method of claim 145, wherein the surgical intervention is functional endoscopic sinus surgery (FESS).

147. The method of claim 145, wherein the steroid is fluticasone propionate or mometasone furoate.

148. The method of claim 145 or 147, wherein the steroid is administered topically, intranasally, systemically, or orally.

149. The method of claim 145, wherein the biologic is dupilumab, omalizumab, benralizumab, reslizumab, or mepolizumab.

150. The method of claim 145, wherein the antibiotic is a macrolide antibiotic.

151. The method of claim 150, wherein the macrolide antibiotic is erythromycin, roxithromycin, azithromycin or clarithromycin.

152. The method of any one of claims 1-151, wherein prior to the administration period is immediately prior to the administration period.

153. The method of any one of claims 1-151, wherein prior to the administration period is from about 1 day to about 14 days prior to the administration period.

154. The method of any one of claims 1-151, wherein prior to the administration period is from about 1 day to about 10 days prior to the administration period.

155. The method of any one of claims 1-151, wherein prior to the administration period is from about 1 day to about 7 days prior to the administration period.

156. The method of any one of claims 1-151, wherein prior to the administration period is from about 1 day to about 4 days prior to the administration period.

157. The method of any one of claims 1-156, wherein the compound of Formula (I), or a pharmaceutically acceptable salt thereof, is the S,S diastereomer:

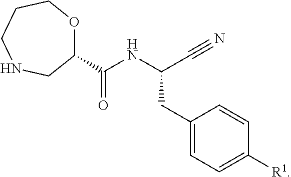

158. The method of any one of claims 1-156, wherein the compound of Formula (I), or a pharmaceutically acceptable salt thereof, is the S,R diastereomer:

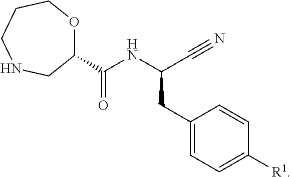

159. The method of any one of claims 1-156, wherein the compound of Formula (I), or a pharmaceutically acceptable salt thereof, is the R,S diastereomer:

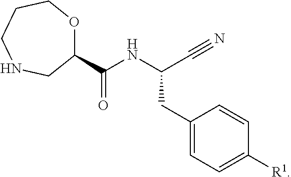

160. The method of any one of claims 1-156, wherein the compound of Formula (I), or a pharmaceutically acceptable salt thereof, is the R,R diastereomer:

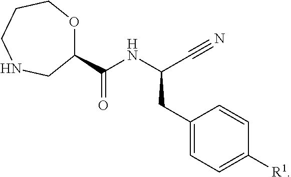

161. The method of any one of claims 1-156, wherein the pharmaceutical composition comprises a mixture of an S,S diastereomer of a compound of Formula (I) and an S,R diastereomer of a compound of Formula (I).

162. The method of any one of claims 1-156, wherein the pharmaceutical composition comprises a mixture of an S,S diastereomer of a compound of Formula (I) and an R,S diastereomer of a compound of Formula (I).

163. The method of any one of claims 1-156, wherein the pharmaceutical composition comprises a mixture of an S,S diastereomer of a compound of Formula (I) and an R,R diastereomer of a compound of Formula (I).

164. The method of any one of claims 1-163, wherein,
R¹ is 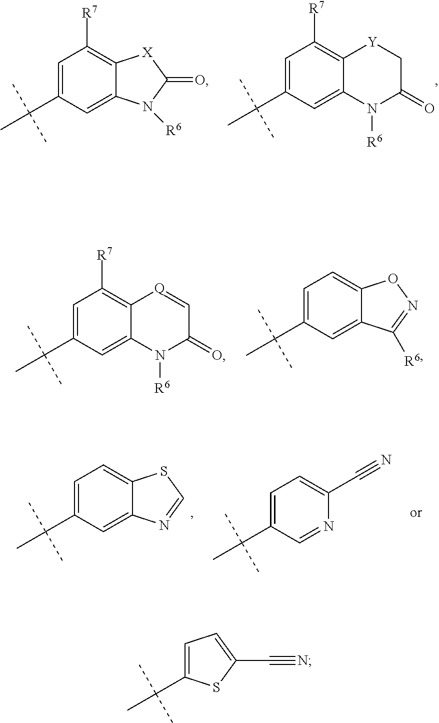
X is O, S or CF₂;
Y is O or S;
Q is CH or N;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

165. The method of any one of claims 1-164, wherein,
R¹ is 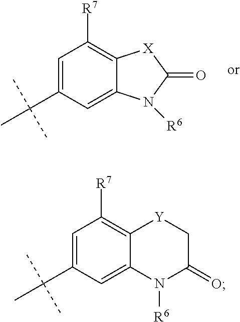
X is O, S or CF₂;
Y is O or S;
R⁶ is C₁₋₃alkyl, wherein said C₁₋₃alkyl is optionally substituted by 1, 2 or 3 F and optionally by one substituent selected from the group consisting of OH, OC₁₋₃alkyl, N(C₁₋₃alkyl)₂, cyclopropyl, and tetrahydropyran; and
R⁷ is hydrogen, F, Cl or CH₃.

166. The method of any one of claims 1-165, wherein, R¹ is

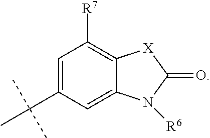

167. The method of claim 166, wherein X is O; R⁶ is C₁₋₃alkyl; and R⁷ is hydrogen.

168. The method of any one of claims 1-165, wherein,
R¹ is R 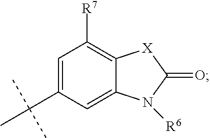
X is O;
R⁶ is C₁₋₃alkyl, wherein said C₁₋₃alkyl is optionally substituted by 1, 2 or 3 F; and
R⁷ is hydrogen.

169. The method of any one of claims 1-168, wherein,
R¹ is 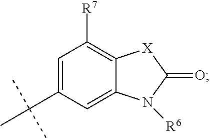
X is O;
R⁶ is C₁₋₃alkyl; and
R⁷ is hydrogen.

170. The method of claim 157, wherein the compound of Formula (I) is (2S)-N-{(1S)-1-cyano-2-[4-(3-methyl-2-oxo-2,3-dihydro-1,3-benzoxazol-5-y-1)phenyl]ethyl}-1,4-oxazepane-2-carboxamide 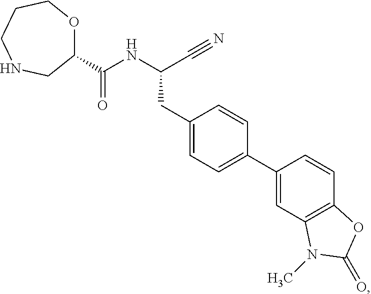 [SMILES: Cn1c(=O)oc2ccc(-c3ccc(C[C@@H](C#N)NC(=O)[C@@H]4CNCCCO4)cc3)cc21]
or a pharmaceutically acceptable salt thereof.

171. The method of claim 157, wherein the compound of Formula (I) is (2S)-N-{(1S)-1-cyano-2-[4-(3-methyl-2-oxo-2,3-dihydro-1,3-benzoxazol-5-y-1)phenyl]ethyl}-1,4-oxazepane-2-carboxamide:

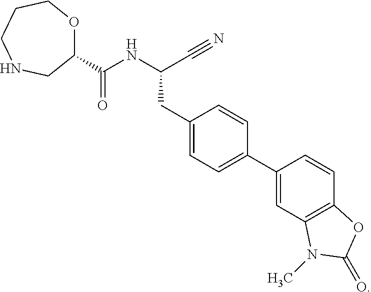 [SMILES: Cn1c(=O)oc2ccc(-c3ccc(C[C@@H](C#N)NC(=O)[C@@H]4CNCCCO4)cc3)cc21]

172. The method of claim 157, wherein the compound of Formula (I) is a hydrate of (2S)-N-{(1S)-1-cyano-2-[4-(3-methyl-2-oxo-2,3-dihydro-1,3-benzoxazol-5-y-1)phenyl]ethyl}-1,4-oxazepane-2-carboxamide:

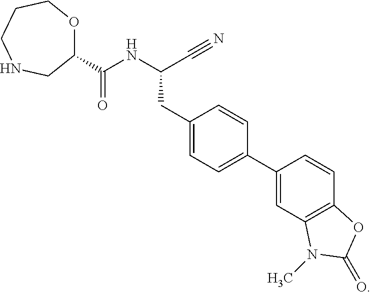 [SMILES: Cn1c(=O)oc2ccc(-c3ccc(C[C@@H](C#N)NC(=O)[C@@H]4CNCCCO4)cc3)cc21]

173. The method of claim 157, wherein the compound of Formula (I) is selected from the group consisting of
(2S)-N-[(1S)-1-Cyano-2-(4′-cyanobiphenyl-4-yl)ethyl]-1,4-oxazepane-2-carboxamide;
(2S)-N-{(1S)-1-Cyano-2-[4-(3-methyl-2-oxo-2,3-dihydro-1,3-benzoxazol-5-yl)phenyl]ethyl}-1,4-oxazepane-2-carboxamide;
(2S)-N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide;
4′-[(2S)-2-Cyano-2-{[(2S)-1,4-oxazepan-2-ylcarbonyl]amino}ethyl]biphenyl-3-yl methanesulfonate;
(2S)-N-{(1S)-1-Cyano-2-[4-(3-methyl-1,2-benzoxazol-5-yl)phenyl]ethyl}-1,4-oxazepane-2-carboxamide;
(2S)-N-{(15)-1-Cyano-2-[4′-(trifluoromethyl)biphenyl-4-yl]ethyl}-1,4-oxazepane-2-carboxamide;
(2S)-N-[(15)-1-Cyano-2-(3′,4′-difluorobiphenyl-4-yl)ethyl]-1,4-oxazepane-2-carboxamide;
(2S)-N-{(15)-1-Cyano-2-[4-(6-cyanopyridin-3-yl)phenyl]ethyl}-1,4-oxazepane-2-carboxamide;
(2S)-N-{(15)-1-Cyano-2-[4-(4-methyl-3-oxo-3,4-dihydro-2H-1,4-benzothiazin-6-yl)phenyl]ethyl}-1,4-oxazepane-2-carboxamide;
(2S)-N-{(15)-1-Cyano-2-[4-(3-ethyl-7-methyl-2-oxo-2,3-dihydro-1,3-benzoxazol-5-yl)phenyl]ethyl}-1,4-oxazepane-2-carboxamide;
(2S)-N-[(15)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide;
(2S)-N-[(15)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide;
(2S)-N-[(15)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide;
(2S)-N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide;
(2S)-N-{(15)-1-Cyano-2-[4-(7-fluoro-3-methyl-2-oxo-2,3-dihydro-1,3-benzoxazol-5-yl)phenyl]ethyl}-1,4-oxazepane-2-carboxamide;
(2S)-N-{(15)-1-Cyano-2-[4-(3-ethyl-2-oxo-2,3-dihydro-1,3-benzoxazol-5-yl)phenyl]ethyl}-1,4-oxazepane-2-carboxamide;
(2S)-N-[(15)-1-Cyano-2-{4-[3-(cyclopropylmethyl)-2-oxo-2,3-dihydro-1,3-benzoxazol-5-yl]phenyl}ethyl]-1,4-oxazepane-2-carboxamide;
(2S)-N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide;
(2S)-N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide;
(2S)-N-{(1S)-1-Cyano-2-[4-(4-methyl-3-oxo-3,4-dihydro-2H-1,4-benzoxazin-6-yl)phenyl]ethyl}-1,4-oxazepane-2-carboxamide;
(2S)-N-[(15)-1-Cyano-2-{4-[3-(2-methoxyethyl)-2-oxo-2,3-dihydro-1,3-benzoxazol-5-yl]phenyl}ethyl]-1,4-oxazepane-2-carboxamide;
(2S)-N-{(1S)-1-Cyano-2-[4-(5-cyanothiophen-2-yl)phenyl]ethyl}-1,4-oxazepane-2-carboxamide;
(2S)-N-[(1S)-2-(4′-Carbamoyl-3′-fluorobiphenyl-4-yl)-1-cyanoethyl]-1,4-oxazepane-2-carboxamide;
(2S)-N-{(1S)-1-Cyano-2-[4-(1-methyl-2-oxo-1,2-dihydroquinolin-7-yl)phenyl]ethyl}-1,4-oxazepane-2-carboxamide;
(2S)-N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide;
(2S)-N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide;
(2S)-N-[(15)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide;
(2S)-N-[(15)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide;
(2S)-N-{(15)-1-Cyano-2-[4-(3-methyl-2-oxo-2,3-dihydro-1,3-benzothiazol-5-yl)phenyl]ethyl}-1,4-oxazepane-2-carboxamide;
(2S)-N-{(15)-1-Cyano-2-[4′-(methylsulfonyl)biphenyl-4-yl]ethyl}-1,4-oxazepane-2-carboxamide;
(2S)-N-{(15)-2-[4′-(Azetidin-1-ylsulfonyl)biphenyl-4-yl]-1-cyanoethyl}-1,4-oxazepane-2-carboxamide;
(2S)-N-[(15)-1-Cyano-2-(4′-fluorobiphenyl-4-yl)ethyl]-1,4-oxazepane-2-carboxamide;
(2S)-N-{(15)-2-[4-(1,3-Benzothiazol-5-yl)phenyl]-1-cyanoethyl}-1,4-oxazepane-2-carboxamide;
(2S)-N-[(15)-1-Cyano-2-(4′-cyanobiphenyl-4-yl)ethyl]-1,4-oxazepane-2-carboxamide;
and pharmaceutically acceptable salts thereof.

174. The method of claim 158, wherein the compound of Formula (I) is (2S)-N-{(1R)-1-Cyano-2-[4-(3-methyl-2-oxo-2,3-dihydro-1,3-benzoxazol-5-yl)phenyl]ethyl}-1,4-oxazepane-2-carboxamide: 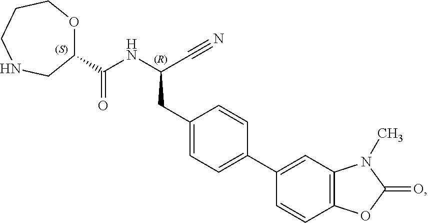
or a pharmaceutically acceptable salt thereof.

175. The method of claim 159, wherein the compound of Formula (I) is (2R)-N-{(1S)-1-Cyano-2-[4-(3-methyl-2-oxo-2,3-dihydro-1,3-benzoxazol-5-yl)phenyl]ethyl}-1,4-oxazepane-2-carboxamide: 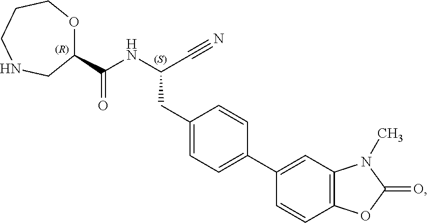
or a pharmaceutically acceptable salt thereof.

176. The method of claim 160, wherein the compound of Formula (I) is (2R)-N-{(1R)-1-Cyano-2-[4-(3-methyl-2-oxo-2,3-dihydro-1,3-benzoxazol-5-yl)phenyl]ethyl}-1,4-oxazepane-2-carboxamide: 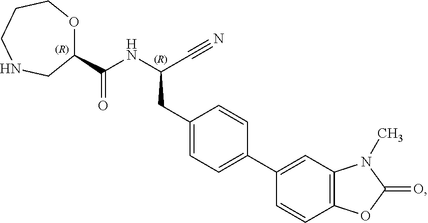
or a pharmaceutically acceptable salt thereof.

177. The method of any one of claims 1-156, wherein the pharmaceutical composition comprises a mixture of brensocatib, or a pharmaceutically acceptable salt thereof and (2S)-N-{(1R)-1-Cyano-2-[4-(3-methyl-2-oxo-2,3-dihydro-1,3-benzoxazol-5-yl)phenyl]ethyl}-1,4-oxazepane-2-carboxamide: 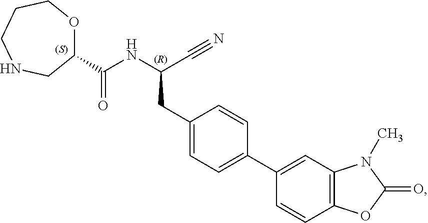
or a pharmaceutically acceptable salt thereof.

178. The method of any one of claims 1-156, wherein the pharmaceutical composition comprises a mixture of brensocatib, or a pharmaceutically acceptable salt thereof and (2R)-N-{(1S-1-Cyano-2-[4-(3-methyl-2-oxo-2,3-dihydro-1,3-benzoxazol-5-yl)phenyl]ethyl}-1,4-oxazepane-2-carboxamide; 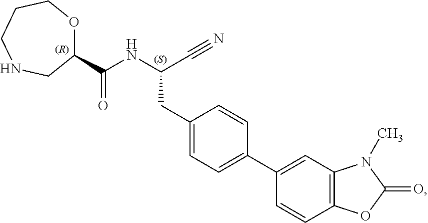
or a pharmaceutically acceptable salt thereof.

179. The method of any one of claims 1-156, wherein the pharmaceutical composition comprises a mixture of brensocatib, or a pharmaceutically acceptable salt thereof and (2R)-N-{(1R)-1-Cyano-2-[4-(3-methyl-2-oxo-2,3-dihydro-1,3-benzoxazol-5-yl)phenyl]ethyl}-1,4-oxazepane-2-carboxamide: 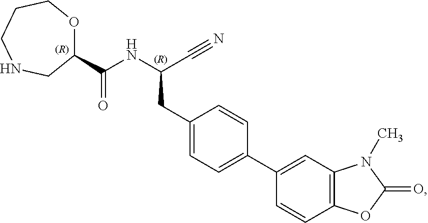
or a pharmaceutically acceptable salt thereof.

180. The method of any one of claims 1-179, wherein the pharmaceutical composition is administered once-a-day, twice-a-day, or every other day during the administration period.

181. The method of any one of claims 1-180, wherein the pharmaceutical composition is administered once-a-day during the administration period.

182. The method of any one of claims 1-181, wherein the pharmaceutical composition is administered orally during the administration period.

183. The method of any one of claims 1-182, wherein the pharmaceutical composition is administered parenterally, enterally, or through a nasogastric tube during the administration period.

184. The method of any one of claims 1-183, wherein the compound of Formula (I) is present in the pharmaceutical composition from about 1 mg to about 100 mg.

185. The method of claim 184, wherein the compound of Formula (I) is present in the pharmaceutical composition at about 10 mg.

186. The method of claim 184, wherein the compound of Formula (I) is present in the pharmaceutical composition at about 25 mg.

187. The method of claim 184, wherein the compound of Formula (I) is present in the pharmaceutical composition at about 40 mg.

188. The method of claim 184, wherein the compound of Formula (I) is present in the pharmaceutical composition at about 5 mg to about 50 mg.

189. The method of claim 184, wherein the compound of Formula (I) is present in the pharmaceutical composition at about 10 mg to about 40 mg.

190. The method of claim 184, wherein the compound of Formula (I) is present in the pharmaceutical composition at about 10 mg to about 25 mg.

191. The method of any one of claims 1-190, wherein the administration period is about 30 days, about 1 month, about 2 months, about 3 months, about 4 months, about 5 months, about 6 months, about 7 months, about 8 months, about 9 months, about 10 months, about 11 months, about 12 months, about 18 months, about 24 months, about 30 months, about 36 months, about 4 years, about 5 years, about 10 years, about 15 years or about 20 years.

192. The method of any one of claims 1-190, wherein the administration period is at least about 30 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 18 months, at least about 24 months, at least about 30 months, at least about 36 months, at least about 4 years, at least about 5 years, at least about 10 years, at least about 15 years or at least about 20 years.

193. The method of any one of claims 1-190, wherein the administration period is about 6 months.

194. The method of any one of claims 1-190, wherein the administration period is about 12 months.

195. The method of any one of claims 1-190, wherein the administration period is from about 6 months to about 36 months.

196. The method of any one of claims 1-190, wherein the administration period is from about 12 months to about 36 months.

197. The method of any one of claims 1-190, wherein the administration period is from about 18 months to about 36 months.

198. The method of any one of claims 1-190, wherein the administration period is from about 1 year to about 50 years.

199. The method of claim 198, wherein the administration period is from about 1 year to about 40 years.

200. The method of claim 198, wherein the administration period is from about 1 year to about 30 years.

201. The method of claim 198, wherein the administration period is from about 1 year to about 25 years.

202. The method of claim 198, wherein the administration period is from about 1 year to about 20 years.

203. The method of claim 198, wherein the administration period is from about 1 year to about 15 years.

204. The method of claim 198, wherein the administration period is from about 1 year to about 10 years.

205. The method of claim 198, wherein the administration period is from about 1 year to about 5 years.

206. The method of claim 198, wherein the administration period is from about 1 year to about 3 years.

207. The method of claim 198, wherein the administration period is from about 1 year to about 2 years.

208. The method of claim 198, wherein the administration period is from about 2 years to about 15 years.

209. The method of claim 198, wherein the administration period is from about 2 years to about 10 years.

210. The method of claim 198, wherein the administration period is from about 2 years to about 8 years.

211. The method of claim 198, wherein the administration period is from about 2 years to about 5 years.

212. The method of claim 198, wherein the administration period is from about 2 years to about 4 years.
